(19) United States
(12) Patent Application Publication
(10) Pub. No.: US 2015/0024415 A1
(43) Pub. Date: Jan. 22, 2015

(54) DETECTION AND QUANTIFICATION OF ANALYTES BASED ON SIGNAL INDUCED BY ALKALINE PHOSPHATE

(71) Applicant: The Board of Trustees of the University of Illinois, Urbana, IL (US)

(72) Inventors: Yi Lu, Champaign, IL (US); Yu Xiang, Urbana, IL (US)

(73) Assignee: The Board of Trustees of the University of Illinois, Urbana, IL (US)

(21) Appl. No.: 14/379,089

(22) PCT Filed: Feb. 19, 2013
(86) PCT No.: PCT/US13/26686

§ 371 Date: Aug. 15, 2014

Related U.S. Application Data
(60) Provisional application No. 61/599,626, filed on Feb. 16, 2012.

(51) Int. Cl. G01N 33/58

(52) U.S. Cl. CPC G01N 33/581; G01N 2458/00
USPC 435/7.72; 435/14; 435/287.1; 435/287.2

(57) ABSTRACT

A general methodology for highly sensitive and selective sensors that can achieve portable, low-cost and quantitative detection of a broad range of targets using only a personal glucose meter (PGM) is disclosed. The method and sensors take advantage of the ability of alkaline phosphatase (ALP) to convert glucose-1-phosphate to glucose, and the ability of PGMs to detect the generated glucose. The disclosed sensors can be part of a lateral flow device. Methods of using such sensors for detecting target agents, for example to diagnose disease, are also provided.

### CROSS REFERENCE TO RELATED APPLICATION

This application claims priority to U.S. Provisional Application No. 61/599,626 filed Feb. 16, 2012, herein incorporated by reference.

### ACKNOWLEDGMENT OF GOVERNMENT SUPPORT

This invention was made with government support under DE-FG02-08ER64568 awarded by the US Department of Energy, under ES 16865 awarded by the National Institutes of Health, and CTS-0120978 awarded by the National Science Foundation. The government has certain rights in the invention.

### FIELD

This application relates to sensors, kits that include such sensors, and methods for making and using such sensors. The sensors and methods take advantage of the ability of alkaline phosphatase to convert glucose-1-phosphate to glucose, and permit detection of a broad array of target agents, and can be used in combination with personal glucose meters.

### BACKGROUND

Developing portable and low cost methods for the detection of targets related environment and health can facilitate the public to monitor environment pollutants and diagnosis diseases at home or in the field, enabling quick responses to the hazards to reduce the risk.^{1,2} Although the detection of some target agents can be efficiently accomplished in the laboratories of research institutes and medical centers, they generally require high cost and long time lag for the public to send the samples and wait for the result. Therefore, many simple and efficient sensors have been developed in to detect many targets of interest, for potential point-of-care applications by the public.³⁻¹⁷ However, to achieve quantitative measurements instead of semi-quantitative or qualitative assays (for example, colorimetric tests based on eye observation), most methods still require laboratory-based devices, such as spectrometers and electrochemical workstations or customized sensor chips that are not widely available to the public.

To overcome these limitations and take the advantage of personal glucose meters (PGMs), general methodologies were previously developed to link PGMs with functional DNA sensors, DNAs and antibodies for quantifying a broad range of non-glucose targets related to health and environment.¹⁸ In addition to these methods, provided herein is a new methodology to use PGMs to monitor disease-related enzyme targets based on the ability of alkaline phosphatase (ALP) to convert glucose-1-phosphate to glucose.

### SUMMARY

The present application discloses methods and sensors that can be used to detect one or more target agents. For example, the methods and sensors can be used to diagnose a disease or condition correlated to the presence or absence of the target.

In one example the method is for detecting a target agent, such as a target enzyme or enzyme activity. In such an example, the method can include contacting a test sample with one or more starting products. A least one of the starting products is one that can be converted to glucose-1-phosphate by a target enzyme, is an enzyme(s) involved in the production of glucose-1-phosphate, or combinations thereof. For example, the target enzyme can be galactose-1-phosphate uridylyltransferase (GALT) and the one or more starting products can be UDP-glucose and galactose-1-phosphate; the target enzyme can be glycogen phosphorylase (GP) and the one or more starting products can be glycogen and 5′-adenosine monophosphate (AMP); the target enzyme can be galactokinase and the one or more starting products can be α-D-galactose, UDP-glucose, and GALT; or the target enzyme can be UDP-glucose 4-epimerase and the one or more starting products can include UDP-galactose, galactose-1-phosphate and GALT. The reaction is incubated under conditions wherein the target enzyme can convert the at least one starting product to glucose-1-phosphate. The resulting glucose-1-phosphate is contacted with alkaline phosphatase (ALP) under conditions that allow the ALP to convert the glucose-1-phosphate to glucose. The resulting glucose is detected with a glucose meter, such as a personal glucose meter (PGM). The target enzyme or activity is detected by correlating the glucose detected, for example wherein an amount of glucose detected corresponds to an amount of target enzyme in the sample.

In one example the disclosed methods can be used to detect one or more target agents, such as at least 2, at least 3, at least 5, at least 10, or at least 20 different targets in a sample, such as 2, 3, 4, 5, 6, 7, 8, 9, 10, 15, 20, 25, 30, 40, 50, 75 or 100 different targets. In one example the disclosed methods can be used to detect one or more target agents, in a plurality of samples simultaneously or contemporaneously, for example as at least 2, at least 3, at least 5, at least 10, at least 20, at least 100, or at least 200 different samples, such as 2, 3, 4, 5, 6, 7, 8, 9, 10, 15, 20, 25, 30, 40, 50, 75, 100, 200, 500, or 1000 different samples.

The method can include contacting a test sample with a first antibody that specifically binds to the target agent. The first antibody is directly or indirectly labeled with ALP, for example with an ALP-conjugated secondary antibody. These agents are incubated under conditions that allow the first antibody and the target agent to bind (and for the ALP-conjugated secondary antibody to bind to the first antibody if present), thereby forming an ALP-first antibody-target agent complex (first complex). The resulting first complex is contacted with a second antibody that specifically binds to the target. These agents are incubated under conditions that allow the second antibody to bind to the target, thereby forming an ALP-first antibody-target agent-second antibody complex (second complex). If used, an ALP-conjugated secondary antibody is specific for the first, but not for the second antibody. In some examples, the second antibody binds to the target on a different epitope than the first antibody. In some examples, the second antibody is immobilized, thereby immobilizing the second complex (due to binding between the target and the second antibody). As a result, the second complex and the ALP that is a part of this complex is not available to react with other agents, such as glucose-1-phosphate. Agents in the mobile phase (e.g., not immobilized), such as ALP-antibody conjugates that did not bind the target or the second antibody, are contacted with glucose-1-phosphate under conditions wherein ALP can convert the glucose-1-phosphate to glucose. The resulting glucose is detected with a glucose meter, such as a PGM. The more target in the sample, the less of the second complex containing the ALP-conjugated secondary antibody there is to interact with the glucose-1-phosphate, therefore giving less glucose signal. The target agent is detected in the sample by correlating the glucose detected wherein an amount of glucose detected inversely corresponds to an amount of target enzyme in the sample.

In one example of a sandwich type assay, the method for detecting one or more targets can include contacting a test sample with an antibody-solid substrate complex, wherein the antibody specifically binds to the target. Examples of solid substrates include plates, lateral flow devices, microfluidic devices, and beads. The antibody-solid substrate complex is incubated under conditions which allow it to bind to the target, if the target is present in the sample. This forms an antibody-solid substrate complex-target complex if the target is present in the test sample. This resulting antibody-solid substrate complex-target agent complex is incubated or contacted with a second antibody specific for the target (which may bind to a different epitope on the target than the antibody on the solid substrate). This results in the formation of an antibody-solid substrate complex-target agent-antibody complex. This resulting antibody-solid substrate complex-target agent-antibody complex is incubated or contacted with a ALP-labeled antibody under conditions which allow it to bind to the second antibody to form an antibody-solid substrate complex-target agent-antibody-ALP-antibody complex. If desired, the antibody-solid substrate complex-target agent-antibody-ALP-antibody complex is separated from a solution in which it is present. The antibody-solid substrate complex-target agent-antibody-ALP-antibody complex is contacted or incubated with glucose-1-phosphate under conditions that permit ALP to convert the glucose-1-phosphate to glucose. The resulting glucose is detected with a personal glucose meter (PGM) and it is determined whether or not the target is present in the sample by correlating the glucose detected.

In one example the method is a competitive assay that can be used to detect one or more targets in a sample. In one example of a competitive assay, target agents compete with ALP-target conjugate analogues in binding with target-specific binding agents (such as antibodies or aptamers), such as those immobilized on a solid support. In the presence of target agents, the ALP-target conjugates are hindered from binding to the immobilized target-specific binding agents, so that they remain in solution. After removal of the solid support by separation, the ALP-target conjugates in solution can catalyze the conversion of glucose-1-phosphate into glucose for PGM measurement. The more target agents in the solution, the more ALP-attached target analogues remain in solution, thus result in higher signal readout in a PGM (“turn on”). For example, the method can include contacting a test sample with a complex that includes target-specific binding agents (such as antibodies or aptamers) immobilized onto a solid support, and with an ALP-target conjugate (ALP conjugate), under conditions that allow the target-specific binding agents-solid substrate complex to bind to the target and to the ALP conjugate. This results in formation of a target-specific binding agent-solid substrate complex-target complex if the target is present in the test sample or formation of a target-specific binding agent-solid substrate complex-ALP conjugate complex if the target is not present in the test sample. Optionally the target-specific binding agent-solid substrate complex-target agent complex or the target-specific binding agent-solid substrate complex-ALP conjugate complex can be separated or removed from a solution in which the target-specific binding agent-solid substrate complex-target agent complex or target-specific binding agent-solid substrate complex-ALP conjugate complex is present. The target-specific binding agent-solid substrate complex-target agent complex, the target-specific binding agent-solid substrate complex-ALP conjugate complex, the solution which contained the target-specific binding agent-solid substrate complex-target agent complex or the solution which contained the target-specific binding agent-solid substrate complex-ALP conjugate complex is incubated or contacted with glucose-1-phosphate under conditions that permit ALP to convert the glucose-1-phosphate to glucose. The resulting glucose is detected with a personal glucose meter (PGM) and a determination made as to whether the target is present or not in the sample by correlating the glucose detected.

In another example of a competitive assay, target agents in a sample can compete with target analogues immobilized on a solid support in binding with ALP-target-specific binding agent conjugates. In the presence of target agents, ALP-target-specific binding agent conjugates are hindered from binding to the immobilized target analogues, but they can bind to the target in the sample, so that they remain in solution. After removal of the solid support by separation, the ALP-target-specific binding agents in solution can catalyze the conversion of glucose-1-phosphate into glucose for PGM measurement. The more target agents in the solution, the more ALP-target-specific binding agents conjugates remain in solution, thus result in higher signal readout in a PGM (“turn on”). For example, the method can include contacting a test sample with a complex that includes the target (such as a analogue of the target or a fragment of the target such as an epitope of the target) immobilized to a solid substrate, and with an ALP-target-specific antibody conjugate (ALP conjugate) under conditions that allow the target to bind to the ALP conjugate and allow the ALP conjugate to bind to the target-solid substrate complex This results in formation of a target-ALP conjugate complex if the target is present in the test sample or formation of a target-solid substrate-ALP conjugate complex if the target is not present in the test sample. Optionally the target-solid substrate complex is separated from a solution in which the target-ALP conjugate complex is present and/o the target-solid substrate-ALP conjugate complex is separated from a solution in which the target-solid substrate-ALP conjugate complex is present. The target-solid substrate complex, the solution containing the target-ALP conjugate complex, the solution which contained the target-solid substrate-ALP conjugate complex and/or the target-solid substrate-ALP conjugate complex are incubated or contacted with glucose-1-phosphate under conditions that permit ALP to convert the glucose-1-phosphate to glucose. This glucose is detected with PGM and a determination made as to whether the target is present or not in the sample by correlating the glucose detected.

In both examples of the competitive assay (the use of ALP-attached target analogues and the use of ALP-attached target-specific antibodies), the ALP-complex on the solid support can also be used to produce glucose from glucose-1-phosphate instead of the ALP-complex in solution. When this alternative method is used, the more target agents in the solution, the less ALP-complex on the solid support, thus resulting in lower signal readout in a PGM (“turn off”).

In one example the method can include contacting a test sample with ALP that is attached to a functional molecule (such as biotin), an agent that binds to the functional molecule (such as avidin, streptavidin or neutravidin if the functional molecule is biotin or a fluorescein antibody if the functional molecule is fluorescein), and first antibody that specifically binds to the target agent, wherein the first antibody is also attached to a functional molecule (for example with biotin or a fluorophore such as fluorescein). In some examples the functional molecule—first antibody, agent that binds to the functional molecule, and functional molecule ALP, are present in a first complex (functional molecule—first antibody—agent that binds to the functional molecule—functional molecule—ALP). These are incubated under conditions that permit the first antibody and the target agent to bind and for the agent that binds to the functional molecule on the first antibody (e.g., streptavidin) to bind to the (e.g., biotinylated) first antibody containing the functional molecule and to the functional molecule (e.g., biotin)-ALP conjugate, thereby forming, for example a functional molecule—first antibody-target agent—agent that binds to the functional molecule—functional molecule—ALP complex (e.g., the second complex). In some examples, the functional molecule and molecule that binds to the functional molecule can be a pair of chemical groups (e.g., COOH and NH₂; ene and thiols), as well as biotin/avidin, biotin/neutravidin, or fluorescein/fluorescein antibody combinations. This resulting complex is incubated with a second antibody that specifically binds to the target agent, under conditions wherein the second antibody and the target agent of the second complex bind, thereby forming a third complex (second antibody-target agent-first antibody—agent that binds to the functional molecule—functional molecule—ALP complex). For example, the first and second antibodies can bind to different epitopes on the target. In some examples, the second antibody is immobilized, such that any of the first complex that binds the target will bind to the immobilized second antibody (generating the third complex). This prevents the third complex from reaching a different region containing glucose-1-phosphate. Only the first complex that did not bind the target (that is, first complex in the mobile phase) is contacted with glucose-1-phosphate under conditions wherein the ALP can convert the glucose-1-phosphate to glucose. The resulting glucose is detected with a glucose meter, such as a PGM. The more target in the sample, the less of the first complex there is to interact with the glucose-1-phosphate, therefore giving less glucose signal. The target agent is detected in the sample by correlating the glucose detected wherein an amount of glucose detected inversely corresponds to an amount of target enzyme in the sample.

Also provided herein are sensors for detecting target agents or target agent activities, for example using the methods provided herein. Exemplary sensors include lateral flow devices. For example, the sensor in one example includes a solid support to which is attached: one or more starting products, wherein at least one of the starting products can be converted to glucose-1-phosphate by a target enzyme, comprises an enzyme involved in the production of glucose-1-phosphate, or combinations thereof; and ALP, wherein the one or more starting products and ALP are attached to different areas of the solid support.

In another example, the sensor includes a solid support to which is attached: a first antibody specific for a target agent; a second antibody specific for the target agent, wherein the first antibody and the second antibody bind to different epitopes of the target agent; a secondary antibody conjugated to ALP, wherein the secondary antibody can specifically bind to the second antibody but not the first antibody; and glucose-1-phosphate. The first antibody, second antibody secondary antibody conjugated to ALP and glucose-1-phosphate are attached to different areas of the solid support.

In another example, the sensor includes a solid support which includes: a first area having attached thereto a first antibody specific for a target agent, an agent that binds to a functional molecule (such as streptavidin), and ALP conjugated to a functional molecule (such as biotin), wherein the first antibody is conjugated to a functional molecule (such as biotin), a second area having attached thereto a second antibody specific for the target agent, wherein the first antibody and second antibody bind to different epitopes of the target agent; and a third area having attached thereto glucose-1-phosphate.

In one example the sensor includes a first solid support with at least three areas (such as membranes). The first area includes an ALP-target conjugate or an ALP-target specific binding agent conjugate. The second area includes either a-target specific binding agent (such as an antibody or aptamer) attached to a second solid support (such as a bead), or having attached thereto a target attached to the second solid support. The third area includes glucose-1-phosphate. The sensor can further optionally have a fourth area that blocks transport of the second solid support.

Also provided are lateral flow devices, microfluidic devices, and kits including the sensors provided herein. Methods of using these sensors, microfluidic devices, and lateral flow devices to detect one or more target agents, for example to diagnose a disease, are provided herein.

The foregoing and other objects and features of the disclosure will become more apparent from the following detailed description, which proceeds with reference to the accompanying figures.

### BRIEF DESCRIPTION OF THE DRAWINGS

FIGS. 1A-1B are schematic drawings showing (A) the equilibrium reaction catalyzed by GALT transforming UDP-glucose and galactose-1-phosphate into UDP-galactose and glucose-1-phosphate, and (B) the production of glucose and galactose from glucose-1-phosphate and galactose-1-phosphate catalyzed by ALP, respectively. In the presence of GALT, the reaction yields glucose-1-phosphate that is further converted to glucose by ALP, thus a signal enhancement is observed in PGMs; while in the absence of GALT, only galactose is produced from galactose-1-phosphate and gives no detectable signal in PGMs.

FIG. 2 is a graph showing the conversion of glucose-1-phosphate into glucose using different concentrations of alkaline phosphatase (ALP) for PGM measurement.

FIG. 3 is a bar graph showing the response of four PGMs to 10 mM glucose (solid bars on left) or 10 mM galactose (striped bars on right).

FIG. 4 is a dot plot showing GALT activities measured in buffer solutions using three different PGMs. The results show that the Bayer Breeze 2 and Optimum Xceed PGMs are selective to glucose and show GALT-dependent signal readouts, while the Accu-Chek Aviva PGM is not selective between glucose and galactose, making these readouts GALT-independent.

FIG. 5 is a graph showing absorbance enhancement of NADPH at 430 nm for the detection of GALT activities using the traditional method.

FIG. 6 is a graph showing the quantitative measurement of GALT activities in human serum using a PGM.

FIG. 7 is a graph showing a comparison of the measurement of GALT activities in human serum in the presence and absence of UDP-glucose and galactose-1-phosphate (Gal-1-P).

FIGS. 8A and 8B are bar graphs showing the effect of (A) GALT- or (B) ALP-catalyzed reaction time on the PGM signals obtained in the GALT assays in human serum. (A) the ALP-catalyzed reaction time is 40 min. (B) The GALT-catalyzed reaction time is 100 min.

FIG. 9 is a graph showing measurement of GALT activities in nonlysed calf blood (spiked by GALT) using a PGM. The signal below the dashed line represents <10 mg/dL and shows as “Low” in the PGM.

FIG. 10 is a graph showing measurement of GALT activity in lysed horse blood (non-spiked) using a PGM.

FIG. 11 is a graph showing measurement of GALT activities in lysed horse blood (spiked by GALT) using a PGM.

FIG. 12 is a schematic drawing showing the PGM-based Hb A1c immunoassay.

FIG. 13 is a graph showing the detection of Hb A1c using a PGM.

FIG. 14 is a dot plot showing GP activities measured in buffer solutions using a PGM.

FIG. 15 is a schematic drawing showing an exemplary lateral flow device that includes ALP for the detection of a target agent in a sample.

FIG. 16 is a schematic drawing showing an exemplary lateral flow device that includes ALP-conjugated secondary antibodies and an immobilized target antibody for the detection of a target agent that specifically binds to the antibody in a sample.

FIG. 17 is a schematic drawing showing an exemplary lateral flow device that includes ALP-conjugated antibodies for the detection of a target agent that specifically binds to the antibody in a sample.

FIG. 18 is a schematic drawing showing an exemplary microfluidic device that includes regions and mixing chambers that permit detection of a target enzyme in the sample.

FIGS. 19A and B are schematic drawings showing competitive assays using (A) Ab-magnetic beads (MB), or (B) target-MB, for turn on/off detection of a target using a glucose meter.

FIG. 20 is a schematic drawing showing an exemplary lateral flow device that can be used for a competitive assay.

### DETAILED DESCRIPTION

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. “Comprising” means “including.” Hence “comprising A or B” means “including A” or “including B” or “including A and B.”

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999.

All publications, patent applications, patents, and other references mentioned herein are incorporated by reference in their entirety for all purposes. All sequences associated with the GenBank Accession numbers mentioned herein are incorporated by reference in their entirety as were present on Feb. 16, 2012. Although exemplary GENBANK numbers are listed herein, the disclosure is not limited to the use of these sequences. Many other enzyme sequences are publicly available, and can thus be readily used in the disclosed methods. In one example, an enzyme having at least 70%, at least 75%, at least 80%, at least 85%, at least 90%, at least 95%, at least 98%, or at least 100% sequence identity to any of the GENBANK numbers are listed herein.

In order to facilitate review of the various embodiments of the disclosure, the following explanations of specific terms are provided:

Alkaline phosphatase (ALP): (EC 3.1.3.1) An enzyme that catalyzes the hydrolysis of phosphate from many phosphorylated biomolecules such as proteins, nucleic acids, lipids and saccharides, including glucose-1-phosphate. In humans, there are three ALP isozymes: ALPI (intestinal), ALPL (tissue non-specific, liver/bone/kidney) and ALPP (placental). Nucleic acid and protein sequences for ALP are publicly available. For example, GENBANK® Accession Nos.: NM_{—}001177520.1 (human), AP009048.1 (E. coli), and NM_{—}176858.2 (cow) disclose exemplary ALP nucleic acid sequences, and GENBANK® Accession Nos.: AAB59378.1 (human); BAE76164.1 (E. coli) and DAA32263.1 (cow) disclose exemplary ALP protein sequences, all of which are incorporated by reference as provided by GENBANK® on Feb. 16, 2012. In certain examples, ALP has at least 80% sequence identity, for example at least 85%, 90%, 95%, or 98% sequence identity to a publicly available ALP sequence, and is an ALP which can catalyze the removal of phosphate groups from glucose-1-phosphate.

Antibody (Ab): A polypeptide that includes at least a light chain or heavy chain immunoglobulin variable region and specifically binds an epitope of an antigen (such as a target agent). Antibodies include monoclonal antibodies, polyclonal antibodies, or fragments of antibodies as well as others known in the art. In some examples, an antibody is specific for a target agent, such as a microbial antigen, spore, cell-surface receptor, or toxin, and thus can be used in the sensors provided herein.

Antibodies are composed of a heavy and a light chain, each of which has a variable region, termed the variable heavy (VH) region and the variable light (VL) region. Together, the VH region and the VL region are responsible for binding the antigen recognized by the antibody. This includes intact immunoglobulins and the variants and portions of them well known in the art, such as Fab′ fragments, F(ab)′2 fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes recombinant forms such as chimeric antibodies (for example, humanized murine antibodies) and heteroconjugate antibodies (such as, bispecific antibodies). See also, Pierce Catalog and Handbook, 1994-1995 (Pierce Chemical Co., Rockford, Ill.); Kuby, Immunology, 3rd Ed., W.H. Freeman & Co., New York, 1997.

A “monoclonal antibody” is an antibody produced by a single clone of B lymphocytes or by a cell into which the light and heavy chain genes of a single antibody have been transfected. Monoclonal antibodies are produced by methods known to those of ordinary skill in the art, for instance by making hybrid antibody-forming cells from a fusion of myeloma cells with immune spleen cells. These fused cells and their progeny are termed “hybridomas.” Monoclonal antibodies include humanized monoclonal antibodies.

Antigen: A molecule that stimulates an immune response. Antigens are usually proteins or polysaccharides. An epitope is an antigenic determinant, that is, particular chemical groups or peptide sequences on a molecule that elicit a specific immune response. An antibody binds a particular antigenic epitope. The binding of an antibody to a particular antigen or epitope of an antigen can be used to determine if a particular antigen (such as a target antigen or antigen of interest) is present in a sample.

Aptamer: An oligonucleotide or peptide molecule that binds with high specificity to a specific target molecule. In one example, aptamers are nucleic acids (such as DNA or RNA) that recognize targets with high affinity and specificity. Aptazymes (also called allosteric DNA/RNAzymes or allosteric (deoxy) ribozymes) are DNA/RNAzymes regulated by an effector (the target molecule). They typically contain an aptamer domain that recognizes an effector and a catalytic domain. In another example, an aptamer is a peptide, which includes a short variable peptide domain, attached at both ends to a protein scaffold.

In vitro selection methods have been used to obtain aptamers for a wide range of target molecules with exceptionally high affinity, having dissociation constants as high as in the picomolar range (Brody and Gold, J. Biotechnol. 74: 5-13, 2000; Jayasena, Clin. Chem., 45:1628-1650, 1999; Wilson and Szostak, Annu. Rev. Biochem. 68: 611-647, 1999). For example, aptamers have been developed to recognize metal ions such as Zn(II) (Ciesiolka et al., RNA 1: 538-550, 1995) and Ni(II) (Hofmann et al., RNA, 3:1289-1300, 1997); nucleotides such as adenosine triphosphate (ATP) (Huizenga and Szostak, Biochemistry, 34:656-665, 1995); and guanine (Kiga et al., Nucleic Acids Research, 26:1755-60, 1998); co-factors such as NAD (Kiga et al., Nucleic Acids Research, 26:1755-60, 1998) and flavin (Lauhon and Szostak, J. Am. Chem. Soc., 117:1246-57, 1995); antibiotics such as viomycin (Wallis et al., Chem. Biol. 4: 357-366, 1997) and streptomycin (Wallace and Schroeder, RNA 4:112-123, 1998); proteins such as HIV reverse transcriptase (Chaloin et al., Nucleic Acids Research, 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as the anthrax (Bruno and Kiel, Biosensors & Bioelectronics, 14:457-464, 1999).

Binding: An association between two substances or molecules, such as the association of an antibody with an antigen (such as a target protein), the association of an aptamer with its target, the association of a protein with another protein or nucleic acid molecule, or the association between a hapten and an antibody. Binding can be detected by any procedure known to one skilled in the art, for example using the methods provided herein.

One molecule is said to “specifically bind” to another molecule when a particular agent (a “specific binding agent”) can specifically react with a particular target, for example to specifically immunoreact with an antibody, or to specifically bind to a particular target agent. The binding is a non-random binding reaction, for example between an antibody molecule and an antigenic determinant or between an aptamer and its target. Binding specificity of an antibody is typically determined from the reference point of the ability of the antibody to differentially bind the specific antigen and an unrelated antigen, and therefore distinguish between two different antigens, particularly where the two antigens have unique epitopes. An antibody that specifically binds to a particular epitope is referred to as a “specific antibody”.

In particular examples, two compounds are said to specifically bind when the binding constant for complex formation between the components exceeds about 10⁴ L/mol, for example, exceeds about 10⁶ L/mol, exceeds about 10⁸ L/mol, or exceeds about 10¹⁰ L/mol. The binding constant for two components can be determined using methods that are well known in the art.

Detect: To determine if a particular agent is present or absent, and in some example further includes quantification of the agent if detected.

Galactokinase (GALK): (EC 2.7.1.6) An enzyme that facilitates phosphorylation of α-D-galactose to produce galactose-1-phosphate. GALK catalyzes the second step of the Leloir pathway, a metabolic pathway found in most organisms for the catabolism of β-D-galactose to glucose 1-phosphate. Mutations in GALK are associated with galactosemia and galactokinase deficiency. Nucleic acid and protein sequences for GALK are publicly available. For example, GENBANK® Accession Nos.: NM_{—}000154.1 (human); NM_{—}001099381.1 (cow) and NM_{—}016905.2 (mouse) disclose exemplary GALK nucleic acid sequences, and GENBANK® Accession Nos.: P51570.1 (human); NP_{—}415278.1 (E. coli); and NP_{—}058601.2 (mouse) disclose exemplary GALK protein sequences, all of which are incorporated by reference as provided by GENBANK® on Feb. 16, 2012. In certain examples, GALK has at least 80% sequence identity, for example at least 85%, 90%, 95%, or 98% sequence identity to a publicly available GALK sequence, and is a GALK which can facilitate phosphorylation of α-D-galactose to produce galactose-1-phosphate.

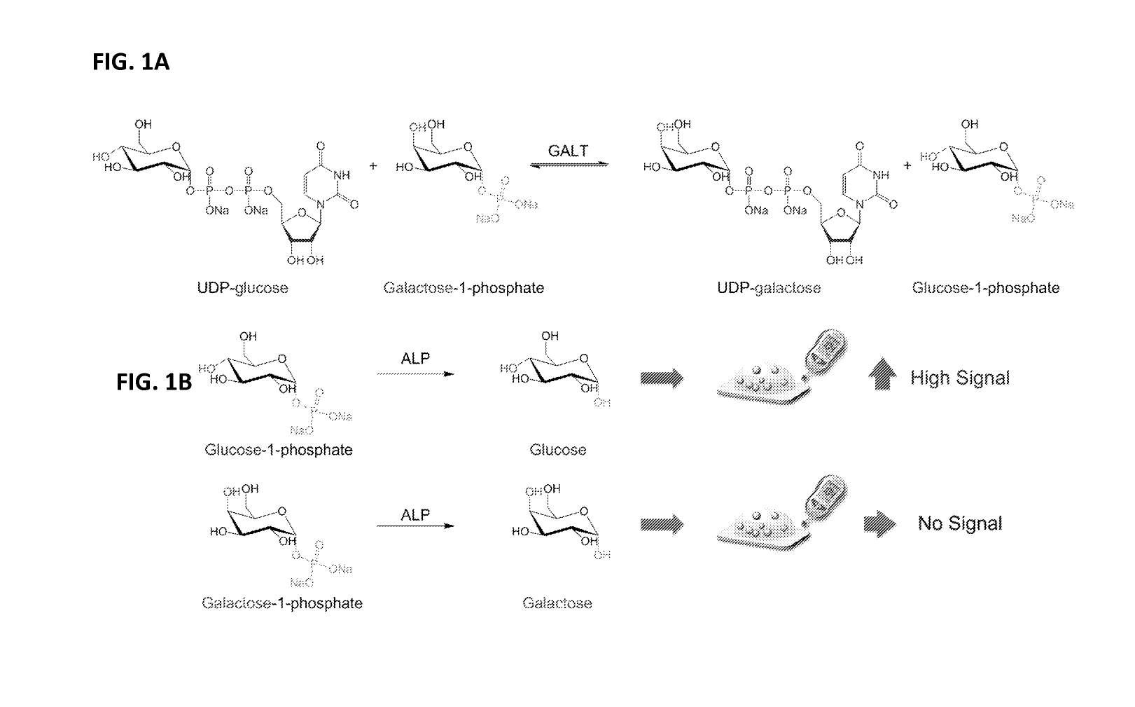

Galactose-1-phosphate uridylyltransferase (GALT): (EC 2.7.7.11) An enzyme that catalyzes the conversion of UDP-glucose and galactose-1-phosphate to produce UDP-galactose and glucose-1-phosphate. The role of GALT enzyme in the galactose metabolism is the transfer of uridine phosphate from uridine diphosphate glucose (UDP-glucose) to galactose-1-phosphate, thereby catalyzing the equilibrium reaction shown in FIG. 1A. Deficiency of GALT causes classic galactosemia. Nucleic acid and protein sequences for GALT are publicly available. For example, GENBANK® Accession Nos.: NM_{—}000146.2 (human) and NP_{—}057867.2 (mouse) disclose exemplary GALT nucleic acid sequences, and GENBANK® Accession Nos.: P07902 (human) and Q3TQJ2 (mouse) disclose exemplary GALT protein sequences, all of which are incorporated by reference as provided by GENBANK® on Feb. 16, 2012. In certain examples, GALT has at least 80% sequence identity, for example at least 85%, 90%, 95%, or 98% sequence identity to a publicly available GALT sequence, and is a GALT which can catalyze the conversion of UDP-glucose and galactose-1-phosphate to produce UDP-galactose and glucose-1-phosphate.

Glucose Meter: A medical device for determining the approximate concentration of glucose in the blood. Glucose meters include commercially available glucose meters, such as a personal glucose meter (PGM). Such meters typically display the level of glucose in mg/dl or mmol/l. In one example a PGM uses tests strips impregnated with glucose oxidase (GOx; EC 1.1.3.4), but not glucose dehydrogenase (such as glucose dehydrogenase/pyrroloquinolinequinone GDH/PQQ). Examples of PGMs that use GOx include but are not limited to: Bayer Breeze 2®, Medisense Optimum Xeed®, and OneTouch® (such as OneTouch Ultra®, OneTouch Hoizon® or OneTouch Surestep®), while examples of PGMs that use glucose dehydrogenase include but are not limited to: Precision Xtra®; Ascensia Contour®; Accu-Chek Compact®; Freestyle®, Accu-Chek Avia® and Free Style Lite®. In some examples, a PGMs is one integrated into a mobile phone or other portable platform (such as an iPad® or other tablet).³⁰ In one example, the PGM is part of (or can be attached to) a cell phone (for example AgaMatrix Inc. (Salem, N.H.) provides a glucose meter that can be attached to a cell phone (such as an iPhone®), and Glooko (Palo Alto, Calif.) provides products that permit one to transfer a reading from a PGM to a cell phone or other device, such as a tablet).

Glycogen phosphorylase (GP): (EC 2.4.1.1) An enzyme that catalyzes the degradation of glycogen to produce glucose-1-phosphate. There are different isoforms of GP, including liver (OMIM 232700), brain (OMIM 138550), and muscle (OMIM 608455) isoforms. Mutations in GP are associated with McArdle disease (muscle isoform), glycogen storage disease type VI (liver isoform), and gastric cancer (brain isoform). Nucleic acid and protein sequences for GP are publicly available. For example, GENBANK® Accession Nos.: NG_{—}013018.1 (muscle); NG_{—}012796.1 (liver) and NM_{—}002862.3 (brain) disclose exemplary GP nucleic acid sequences, and GENBANK® Accession Nos.: P11217 (muscle); P06737 (liver); and P11216.5 (brain) disclose exemplary GP protein sequences, all of which are incorporated by reference as provided by GENBANK® on Feb. 16, 2012. In certain examples, GP has at least 80% sequence identity, for example at least 85%, 90%, 95%, or 98% sequence identity to a publicly available GP sequence, and is a GP which can catalyze the degradation of glycogen to produce glucose-1-phosphate.

Immobilized: Bound to a surface, such as a solid support. In one embodiment, the solid surface is in the form of a membrane. The surface can include immobilized proteins, such as ALP and antibodies that can specifically bind to a target agent. In some examples, the surface can have attached thereto glucose-1-phosphate. Methods of immobilizing agents to solid supports are known in the art. For example, methods of immobilizing peptides on a solid surface can be found in WO 94/29436, and U.S. Pat. No. 5,858,358. In some examples, agents are immobilized to a support by simply applying the agent in solution to the support, and allowing the solution to dry, thereby immobilizing the agent to the support.

Lateral flow device: An analytical device in the form of a test strip used in lateral flow chromatography, in which a sample fluid, such as one to be tested for the presence of a target agent, flows (for example by capillary action) through the strip (which is frequently made of bibulous materials such as paper, nitrocellulose, and cellulose). The test sample and any suspended target agent(s) can flow along the strip to a detection zone in which glucose produced as a result of the presence or absence of the target agent is detected, to indicate a presence, absence and/or quantity of the target agent.

Numerous lateral flow analytical devices are known, and include those shown in U.S. Pat. Nos. 4,313,734; 4,435,504; 4,775,636; 4,703,017; 4,740,468; 4,806,311; 4,806,312; 4,861,711; 4,855,240; 4,857,453; 4,943,522; 4,945,042; 4,496,654; 5,001,049; 5,075,078; 5,126,241; 5,451,504; 5,424,193; 5,712,172; 6,555,390; 6,368,876; 7,799,554; EP 0810436; and WO 92/12428; WO 94/01775; WO 95/16207; and WO 97/06439, each of which is incorporated by reference.

Lateral flow devices can in one example be a one-step lateral flow assay in which a sample fluid is placed in a sample or wicking area on a bibulous strip (though, non bibulous materials can be used, and rendered bibulous by applying a surfactant to the material), and allowed to migrate along the strip until the sample comes into contact with one or more reagents, that lead to the interaction between glucose-1-phosphate and ALP, for the production of glucose. The resulting glucose can be detected with a PGM.

In some examples, the strip includes multiple regions for detecting different test agents in the sample (for example in parallel lines or as other separate portions of the device). The test strips can also incorporate control indicators, which provide a signal that the test has adequately been performed, even if a positive signal indicating the presence (or absence) of a target is not achieved.

A lateral flow device can include a sample application area or wicking pad, which is where the fluid or liquid sample is introduced. In one example, the sample may be introduced to the sample application area by external application, as with a dropper or other applicator. In another example, the sample application area may be directly immersed in the sample, such as when a test strip is dipped into a container holding a sample. In yet another example, the sample may be applied, blotted, poured or expressed onto the sample application area.

A lateral flow device can include a reagent or conjugation pad, the region of a lateral flow device where reagents are immobilized, such as the starting products (such as those that can be converted to glucose-1-phosphate), ALP, AP-conjugated antibodies, target-specific binding agent-bound solid substrates (such as antibody or aptamer immobilized to magnetic beads), target-bound solid substrates (such as targets immobilized to magnetic beads, Target-MB), ALP-target conjugate (ALP conjugate), ALP-target-specific binding agent conjugate (ALP conjugate), antibodies specific for a target agent, or combinations thereof. A lateral flow device may have more than one conjugation area, for example, a “primary conjugation area,” a “secondary conjugation area,” and so on. Often different reagents are immobilized in the primary, secondary, or other conjugation areas. Multiple conjugation areas may have any orientation with respect to each other on the lateral flow substrate; for example, a primary conjugation area may be distal or proximal to a secondary (or other) conjugation area and vice versa. Alternatively, a primary conjugation area and a conjugation (or other) area may be oriented perpendicularly to each other such that the two (or more) conjugation areas form a cross or a plus sign or other symbol. For example, Apilux et al. (Anal. Chem. 82:1727-32, 2010), Dungchai et al. (Anal. Chem. 81:5821-6, 2009), and Dungchai et al. (Analytica Chemica Acta 674:227-33, 2010), provide exemplary lateral flow devices with a central sample area and one or more conjugation areas distal to the sample area, which provide independent test zones where independent reactions can occur (e.g., each test zone has a different reagents for detecting a particular test agent, and can further include one or more reaction pads where reactions can take place (for example interspersed between the reagent pads) and an absorption pad that receives the generated glucose, wherein each absorption pad can be independently read by a PGM), for example that form a “Y”, cloverleaf, or spoke-wheel pattern.

A lateral flow device can include one or more reaction pads, such as a membrane, that can be a place to allow desired reactions to occur, and an absorption pad that draws the sample across the conjugation pad(s) and membrane(s) by capillary action and collects it.

Sensor: A device used to detect the presence of a target agent or target activity. The disclosed sensors can include ALP (for example alone or conjugated to an antibody, the target, or other molecule) and glucose-1-phosphate attached to a solid support.

Target Agent or activity: A substance or activity whose detection is desired, including, but not limited to, a chemical compound, metal, pathogen, toxin, or protein (such as a cytokine, hormone or antigen), as well as particular cells (such as a cancer cell or bacterial cell), viruses, spores, or particular activities, such as a particular enzyme activity. In one example a target agent can produce glucose-1-phosphate directly or indirectly. In another example, a target is an agent that can bind to an antibody with high specificity.

UDP-glucose 4-epimerase (GALE): (EC 5.1.3.2) An enzyme that performs the final step in the Leloir pathway of galactose metabolism, including catalyzing the reversible conversion of UDP-galactose to UDP-glucose. Mutations in GALE are associated with type III galactosemia. Nucleic acid and protein sequences for GALE are publicly available. For example, GENBANK® Accession Nos.: NM_{—}001127621.1 (human); NP_{—}001193137.1 (cow) and NM_{—}178389.3 (mouse) disclose exemplary GALE nucleic acid sequences, and GENBANK® Accession Nos.: NP_{—}001121093.1 (human); NM_{—}001206208.1 (E. coli); and NP_{—}848476.1 (mouse) disclose exemplary GALE protein sequences, all of which are incorporated by reference as provided by GENBANK® on Feb. 16, 2012. In certain examples, GALE has at least 80% sequence identity, for example at least 85%, 90%, 95%, or 98% sequence identity to a publicly available GALE sequence, and is a GALE which can catalyze the reversible conversion of UDP-galactose to UDP-glucose.

### Overview

Personal glucose meters (PGMs) are currently the most successfully commercialized public meters for portable self-diagnosis. PGMs are simple, inexpensive, quantitative, and widely available for worldwide public use. The successful integration of PGMs with mobile phones allows even wider adoption and better user experience. However, currently, PGMs are only used to help diabetes patients monitor blood glucose. Although other targets can be efficiently detected in research and medical laboratories, they generally require high cost and long time lag for the public to send the samples and wait for the result.

To adapt the PGMs to the detection and quantification of other targets, the present disclosure provides methods and sensors that permit detection (and in some examples quantification) of other targets. Thus, the disclosed methods and sensors enable point-of-care (POC) detection of a wide range of targets. Although previous reports disclosed the use of PGMs as POC devices by using antibodies or aptamers for the detection of some analytes, the lack of a link between an enzymatic activity and glucose production was a gap in those prior PGM-based methodologies, because numerous clinical tests take advantage of enzymatic activities instead of molecular recognition by functional DNAs or antibodies. The sensors and methods take advantage of alkaline phosphatase (ALP),³¹ which can hydrolyze glucose-1-phosphate to produce glucose, and the glucose generated detected using a PGM. As illustrated in FIG. 1B (middle row of the figure), after the transformation of a starting product (such as galactose-1-phosphate) into glucose-1-phosphate by the equilibrium reaction catalyzed by an enzyme (here, GALT is illustrated), ALP can further convert the generated glucose-1-phosphate into glucose, which is then measured by PGMs to assess the target enzyme activity quantitatively. In contrast, no glucose signal is detected in the absence of the target enzyme (for example for GALT, only galactose is formed by ALP, FIG. 1B, bottom row of the figure). Thus, the method and sensors described herein can be adapted to quantify a wide range of analytes, including enzyme activities, using a PGM.

In one example the target is an enzyme that can convert (directly or indirectly) one or more starting materials to one or more products that include glucose-1-phosphase, which is then converted to glucose by alkaline phosphatase (ALP). In some examples, the amount of glucose detected is proportional to the amount of target enzyme or enzyme activity in the test sample. The resulting glucose is then detected with a PGM. Exemplary enzyme activities that can be detected using such methods include galactose-1-phosphate uridylyltransferase (GALT) activity, glycogen phosphorylase (GP), and enzymes involved in galactose metabolism (e.g., galactokinase and UDP-glucose 4-epimerase). In some examples, blood or fractions thereof (such as serum) are used as the samples in which the target activity is detected.

Detection of GALT activity permits diagnosis of classic galactosemia. Screening for classic galactosemia is essential to ensure the safety in consuming food containing lactose or galactose, and it is important for newborns because of their immediate need for milk. Traditional GALT assays are considered the golden standard for the diagnosis of classic galactosemia (Bosch, J. Inherit. Metab. Dis. 2006, 29:516). Currently available GALT activity assays measure the amount of glucose-1-phosphate produced from the reaction catalyzed by GALT (FIG. 1A). Glucose-1-phosphate is transformed into glucose-6-phosphate by phosphoglucomutase and subsequently oxidized by nicotinamide adenine dinucleotide phosphate (NADP⁺) to its reduced form NADPH in the presence of glucose-6-phosphate dehydrogenase (G6PD). The change in the absorbance or fluorescence of NADP⁺ after reduction to NADPH is used to quantify the concentration of glucose-6-phosphate, which is equivalent to that of glucose-1-phosphate produced by the GALT-catalyzed reaction.^{23,32} The quantitative measurements are usually performed using UV-Vis or fluorescence spectrometers. Thus, the traditional GALT assays require two enzymes (e.g., phosphoglucomutase and G6PD) to transform the product of a GALT-catalyzed reaction (glucose-1-phosphate) into detectable signals,^{23,32} and suffer from the interference of intrinsic color of blood samples due to the spectroscopic measurements involved in the assays, so that complicated steps to remove hemoglobin are necessary. In contrast, the disclosed methods only need alkaline phosphatase (ALP) to convert the product of a GALT-catalyzed reaction (glucose-1-phosphate) into PGM-detectable glucose. Furthermore, the GALT activity measurements by PGMs are not affected by the color or turbidity of the samples. The sensitivity of the disclosed method (0.006 U/mL) is sufficient for diagnosis of classic galactosemia (borderline about 0.020 U/mL). With the portability, low cost and wide availability of PGMs, the disclosed method can be used to quantify GALT activities and screen classic galactosemia at home or in the field, thereby avoiding the high cost and long time lag for the similar assays in hospitals and medical centers. In addition, using the disclosed methods and sensors to quantitatively measure GALT activity permits differentiation of galactosemia, carrier, and normal individuals.

Thus, in some examples, the disclosed methods and sensors can be used without the need for laboratory-based instruments or complicated sample pre-treatment.

It is also shown herein that alkaline phosphatase (ALP)-antibody conjugates can be used in an immunoassay (Yalow and Berson, Nature 1959, 184:1648; Kim et al., Anal. Chem. 2009, 81:9183; Thaxton et al., Proc. Nat. Acad. Sci. U.S.A. 2009, 106: 18437; Stoeva et al., J. Am. Chem. Soc. 2006, 128:8378; Mattoussi et al., J. Am. Chem. Soc. 2000, 122:12142; Kingsmore et al., Curr. Opin. Biotechnol. 2003, 14:74; Zhao et al., Trend Anal. Chem. 2009, 28:404; Lin et al., Anal. Biochem. 2005, 341:1) in combination with PGMs for the detection of non-glucose targets. For example, it is shown that glycated hemoglobin (Hb A1c) can be detected using a PGM, which permits diagnosis of diabetes (by Hb A1c instead of blood sugar level). Currently available methods for detecting Hb A1c require patients to go to hospitals or use other meters. Using the disclosed methods and sensors, patients (such as patients with diabetes) can perform blood glucose and Hb A1c measurements using only one glucose meter.

The disclosure provides exemplary sandwich and competitive assays that can be used to detect one or more targets. For example, in a sandwich assay, binding of the target agent to a specific binding agent (such as an antibody or aptamer present on a solid support, such as a bead) is followed by incubating the target-specific binding agent complex with an ALP-labeled target-specific binding agent (wherein the label is direct or indirect, for example by using an ALP-labeled secondary antibody, and wherein the target-specific binding agent recognizes a different epitope of the target than the previously used specific binding agent) under conditions sufficient to allow binding of the ALP-labeled target-specific binding agent to the target bound to the target-specific binding agent. This results in the formation of a “sandwich” type structure, wherein the target-specific binding agent is bound to the solid support and the target, and the ALP is bound (directly or indirectly, for example via an antibody) to the target (and in some examples also the target-specific binding agent). In some examples, the solid support need not be separated or otherwise removed from the ALP prior to detection of glucose. The sandwich complex is contacted with glucose-1-phosphate, thereby generating glucose. The resulting glucose is then detected, for example with a PGM, wherein detection of glucose indicates the presence of the target in the sample, and an absence of detected glucose indicates the absence of the target in the sample.

In some examples, a competitive assay is used to detect one or more targets. Fore example, the target (or a portion thereof, such as an epitope) or target-specific binding agents can be attached to a solid support, such as a bead. The resulting target-solid support or target-specific binding agent-solid support can be contacted with an ALP-target-specific antibody conjugate or ALP-target conjugate (ALP conjugate), respectively. If the target is present, it will compete with the ALP-target conjugate for binding to the target specific binding agent-solid support conjugate. Alternatively, if the target is present, it will bind to the ALP-target-specific antibody conjugate preventing it from binding to the target-solid support. The solid support can be separated from the rest of the reaction, and the resulting solid support or rest of the reaction (e.g., solution) can be incubated with glucose-1-phosphase and glucose produced.

The disclosed sensors and methods therefore permit detection (e.g., quantitative detection) of target agents, such as those related to the environment and health, using commercially available reagents and PGMs, thereby allowing the public or others to conduct assays of target analytes at home and in-field. Thus, this disclosure permits the detection of many different target analytes using a single PGM (for example by using interchangeable lateral flow devices or test strips, each specific for a particular target).

### Methods of Detecting Target Agents

Methods of detecting one or more target agents, for example using the sensors and devices disclosed herein, are provided herein. The disclosed sensors, including lateral flow devices, can be used with the disclosed methods for detecting one or more target agents or activities, for example to diagnose a disease or infection, or to detect exposure to a particular metal or drug. For example, one or more steps of the disclosed methods can be performed by the disclosed sensors, for example by applying the test sample to a sensor that includes one or more reagents needed to perform the steps of the method.

In some examples, for example when the sensor is part of a lateral flow device, the method can include contacting the lateral flow device with a sample under conditions sufficient to allow the target agent in the sample to flow through the lateral flow device and interact with various reagents present on the lateral flow device, including ALP and glucose-1-phosphate, to ultimately produce glucose. The resulting glucose is detected, for example with a PGM. In some examples, detection of glucose indicates the presence of the target agent in the sample, and an absence of detected glucose indicates the absence of the target agent in the sample. In other examples, detection of glucose indicates the absence of the target agent in the sample, and an absence of detected glucose indicates the presence of the target agent in the sample. In some examples, the glucose detected is compared to a control, such as a reference value(s) indicating the presence or absence of the target or samples known to have or not have the target (such as a reference sample containing a known amount of target or a sample known not to contain the target).

Methods are provided for detecting targets that directly or indirectly produce glucose-1-phosphate from one or more starting products. In one example, the method includes contacting one or more sensors (such as a lateral flow device) with a sample under conditions sufficient to allow the target that may be present in the test sample to interact with the one or more starting products (which are immobilized to the solid support). This results in the production of glucose-1-phosphate, which reacts with ALP immobilized to the solid support, thereby producing glucose which can be detected by a PGM (for example by contacting the PGM with the solid support).

Methods are also provided for detecting targets that can specifically bind to an antibody or an aptamer.

In some examples, the disclosed methods include neutralizing the reaction, for example from a basic pH to a neutral pH (for example to a pH of about 6.5 to 7.5, such as pH 6.8 to 7.2), after the glucose is produced, but before it is detected with a glucose meter. For example the reaction can be incubated with a buffer containing phosphate buffers (e.g., sodium, potassium or ammonium salts, such as NaH₂PO₄ and the like), and other buffers such as Tris-HCl, HEPES, MES, NaHCO₃—Na₂CO₃, and the like.

Detecting Target Agents that Produce Glucose-1-Phosphate

In one example, the target is an agent, such as an enzyme, that can convert one or more starting products to glucose-1-phosphate, directly or as an early step in a pathway to produce glucose-1-phosphate. As described above, selecting appropriate starting product(s) permits detection of the target enzyme, and allows one to develop a sensor that can be used to detect a particular target enzyme that can produce glucose-1-phosphate, directly or indirectly. If the target enzyme produces glucose-1-phosphate directly from a starting product (such as galactose-1-phosphate), additional enzymes may not be required. However, if the target enzyme does not produce glucose-1-phosphate directly, but instead produces a product in the pathway that can be converted to glucose-1-phosphate by a second enzyme, the second enzyme (or more enzymes as needed) can be supplied (for example with the starting products).

Methods are provided for detecting a target agent or its activity, such as a target enzyme. In some examples, such an enzyme produces glucose-1-phosphate directly from one or more starting materials (such as galactose-1-phosphate and UDP-glucose). Examples of such enzymes are galactose-1-phosphate uridylyltransferase (GALT) and glycogen phosphorylase (GP). In another example, the target enzyme produces glucose-1-phosphate indirectly from one or more starting materials. For example, the enzyme can be part of a pathway that produces galactose-1-phosphate, UDP-glucose, or both. Examples of such enzymes are galactokinase (GALK) and UDP-glucose 4-epimerase.

In some examples, the method includes contacting a test sample (such as a blood sample or tumor sample) with one or more starting products, wherein at least one of the starting products can be converted to glucose-1-phosphate by the target enzyme or includes an enzyme involved in the production of glucose-1-phosphate. The test sample and one or more starting products are incubated under conditions that allow the target enzyme to convert the at least one starting product to glucose-1-phosphate. The glucose-1-phosphate is contacted with alkaline phosphatase (ALP) under conditions that allow the ALP to convert the glucose-1-phosphate to glucose. The method then includes detecting the glucose produced with a personal glucose meter (PGM), and determining that the target enzyme (or its activity) is present when glucose is detected. Detecting the glucose can be qualitative or quantitative. In some examples, the amount of glucose detected by the PGM is proportional to the amount of target enzyme or activity in the sample.

In one example, the target enzyme is GALT. GALT produces glucose-1-phosphate directly from galactose-1-phosphate and UDP-glucose (see FIG. 1A). Thus, in such an example, the one or more starting products include UDP-glucose and galactose-1-phosphate. In another example, the target enzyme is GP. GP (in combination with 5′-AMP) produces glucose-1-phosphate directly from glycogen (see FIG. 14). Thus, in such an example, the one or more starting products include glycogen and 5′-AMP.

In one example, the target enzyme is GALK. GALK phosphorylates α-D-galactose to produce galactose-1-phosphate, which can then be converted to glucose-1-phosphate in the presence of UDP-glucose and GALT as described above. Thus, in such an example, the one or more starting products include α-D-galactose, UDP-glucose, and GALT (a non-target enzyme in this example). In another example, the target enzyme is UDP-glucose 4-epimerase. UDP-glucose 4-epimerase converts UDP-galactose to UDP-glucose. The UDP-glucose can be used in combination with galactose-1-phosphate and GALT to produce glucose-1-phosphate (see FIG. 1A). Thus, in such an example, the one or more starting products include UDP-galactose, galactose-1-phosphate and GALT.

In particular examples, the sample is obtained from a subject. In such examples, the method can further include obtaining the sample from the subject. In some examples, the method includes selecting a subject having or suspected of having a particular disease associated with the target enzyme. In some examples, the method further includes determining that the subject has a particular disease depending on whether the target enzyme activity is detected or not (or is increased or decreased relative to an appropriate normal control, such as a sample from a subject that does not have the disease screened for or a reference value indicting absence of the test agent). In some examples, the method includes treating the subject for the disease diagnosed using the disclosed methods.

For example, the target enzyme activity can be GALT activity and the disease is classic galactosemia, wherein the subject is determined or diagnosed to have classic galactosemia when GALT activity is significantly reduced or absence not present, for example relative to a normal control. In another example, the target enzyme activity is GP activity and the disease is McArdle disease, glycogen storage disease type VI or gastric cancer (depending on the isoform of GP). The subject is determined to have classic McArdle disease or glycogen storage disease type VI when GP activity is significantly reduced or absent, for example relative to a normal control. The subject is determined to have gastric cancer or colorectal cancer when GP activity is significantly increased, for example relative to a normal control. In another example, the target enzyme activity is galactokinase activity and the disease is galactosemia or galactosemia type II. The subject is determined to have galactosemia or galactosemia type II when galactokinase activity is significantly reduced or absent, for example relative to a normal control. In another example, the target enzyme activity is UDP-glucose 4-epimerase activity and the disease is type III galactosemia, wherein the subject is determined to have type III galactosemia when UDP-glucose 4-epimerase activity is significantly reduced or absent, for example relative to a normal control.

In particular examples, the PGM used is one that uses a test strip that includes glucose oxidase (GOx). Examples of PGMs that use GOx include but are not limited to: Bayer Breeze 2®, Medisense Optimum Xeed®, and OneTouch® (such as OneTouch Ultra®, OneTouch Hoizon® or OneTouch Surestep®). In particular examples, the PGM used is one that does not use a test strip that includes glucose dehydrogenase (such as glucose dehydrogenase/pyrroloquinolinequinone GDH/PQQ).

Detecting Target Agents that Specifically Bind an Antibody or Aptamer

In one example, the target is an agent that can specifically bind to one or more antibodies or aptamers. Although the term antibodies is more commonly used throughout this application, one will appreciate that aptamers can be used instead of, or in combination with the antibodies. By selecting appropriate antibodies (or aptamers) specific for the target agent, permits detection of the target, and allows one to develop a sensor that can be used to detect a particular target. Any target for which there are available antibodies (or aptamers), or for which antibodies (or aptamers) can be routinely developed, are covered by this disclosure. Exemplary antibodies include polyclonal antibodies, monoclonal antibodies, as well as fragments thereof. In some examples the antibody (or aptamer) includes a label, such as an enzyme (such as ALP). Exemplary targets are provided below.

In some examples, the method includes contacting a test sample (such as a blood sample or tumor sample) with a first antibody that specifically binds to the target agent. The first antibody is labeled with ALP, directly or indirectly (for example using a secondary antibody conjugated to (or labeled with) ALP). In some examples, the first antibody is an IgG-ALP, a single chain Fv ALP antibody, or is an ALP nucleic acid aptamer, which can also include nanomaterials such as gold nanoparticles. If used, n ALP-labeled secondary antibody is specific for the first antibody (for example if the first antibody is a rabbit polyclonal antibody, the secondary antibody conjugated to ALP can be an anti-rabbit secondary antibody, such as a goat or mouse anti-rabbit secondary antibody). The test sample and the ALP-first antibody are incubated under conditions that allow specific binding between the first antibody and the target (and between the first antibody and the ALP-labeled secondary antibody if used), thereby forming an ALP-first antibody-target agent complex (first complex). This resulting first complex is contacted with a second antibody that specifically binds to the target agent. The second antibody and the first complex are incubated under conditions that allow specific binding between the target agent present in the first complex and the second antibody, thereby forming an ALP-first antibody-target agent-second antibody complex (second complex). In one example, the second antibody is immobilized (for example on an area of a solid support), such that any target that binds to the second antibody will become immobile, as is the second complex. As a result, the second complex and the ALP that is a part of this complex is not available to react with other agents, such as glucose-1-phosphate. Agents in the mobile phase, such as ALP-first antibody that did not bind the target, are contacted with glucose-1-phosphate under conditions wherein ALP can convert the glucose-1-phosphate to glucose. The resulting glucose is detected with a glucose meter, such as a PGM. The more target in the sample, the less of the ALP and second complex there is to interact with the glucose-1-phosphate, therefore giving less glucose signal. The target agent is detected in the sample by correlating the glucose detected wherein an amount of glucose detected inversely corresponds to an amount of target enzyme in the sample. Detecting the glucose can be qualitative or quantitative.

In some examples, the method includes contacting a test sample (such as a biological, environmental or food sample) with ALP that is attached to a functional molecule (such as biotin), an agent that binds to the functional molecule (such as avidin, streptavidin or neutravidin if the functional molecule is biotin or a fluorescein antibody if the functional molecule is fluorescein), and first antibody that specifically binds to the target agent, wherein the first antibody is also attached to a functional molecule (for example with biotin or a fluorophore such as fluorescein). In some examples the functional molecule—first antibody, agent that binds to the functional molecule, and functional molecule ALP, are present in a first complex (functional molecule—first antibody—agent that binds to the functional molecule—functional molecule—ALP). These are incubated under conditions that permit the first antibody and the target agent to bind and for the agent that binds to the functional molecule on the first antibody (e.g., streptavidin) to bind to the (e.g., biotinylated) first antibody containing the functional molecule and to the functional molecule (e.g., biotin)-ALP conjugate, thereby forming, for example a functional molecule—first antibody-target agent—agent that binds to the functional molecule—functional molecule—ALP complex (e.g., the second complex). In some examples, the functional molecule and molecule that binds to the functional molecule can be a pair of chemical groups (e.g., COOH and NH₂; ene and thiols), as well as biotin/avidin, biotin/neutravidin, or fluorescein/fluorescein antibody combinations. This resulting complex is incubated with a second antibody that specifically binds to the target agent, under conditions wherein the second antibody and the target agent of the second complex bind, thereby forming a third complex (second antibody-target agent-first antibody—agent that binds to the functional molecule—functional molecule—ALP complex). For example, the first and second antibodies can bind to different epitopes on the target. In some examples, the second antibody is immobilized, such that any of the first complex that binds the target will bind to the immobilized second antibody (generating the third complex). This prevents the third complex from reaching a different region containing glucose-1-phosphate. Only the first complex that did not bind the target is contacted with glucose-1-phosphate under conditions wherein the ALP can convert the glucose-1-phosphate to glucose. The resulting glucose is detected with a glucose meter, such as a PGM. The more target in the sample, the less of the first complex there is to interact with the glucose-1-phosphate, therefore giving less glucose signal. The target agent is detected in the sample by correlating the glucose detected wherein an amount of glucose detected inversely corresponds to an amount of target enzyme in the sample. Detecting the glucose can be qualitative or quantitative.

In particular examples, the sample is obtained from a subject. In such examples, the method can further include obtaining the sample from the subject. In some examples, the method includes selecting a subject having or suspected of having a particular disease associated with the target agent. In some examples, the method further includes determining that the subject has a particular disease depending on whether the target is detected or not (or is increased or decreased relative to an appropriate normal control, such as a sample from a subject that does not have the disease screened for).

### Exemplary Competitive Assays

Turn on and turn off competitive assay methods can be used to detect a target, such as a target of interest having only one binding site (e.g., some small molecular targets). Thus, in some examples a competitive assay method is used to detect a mono-epitope target.

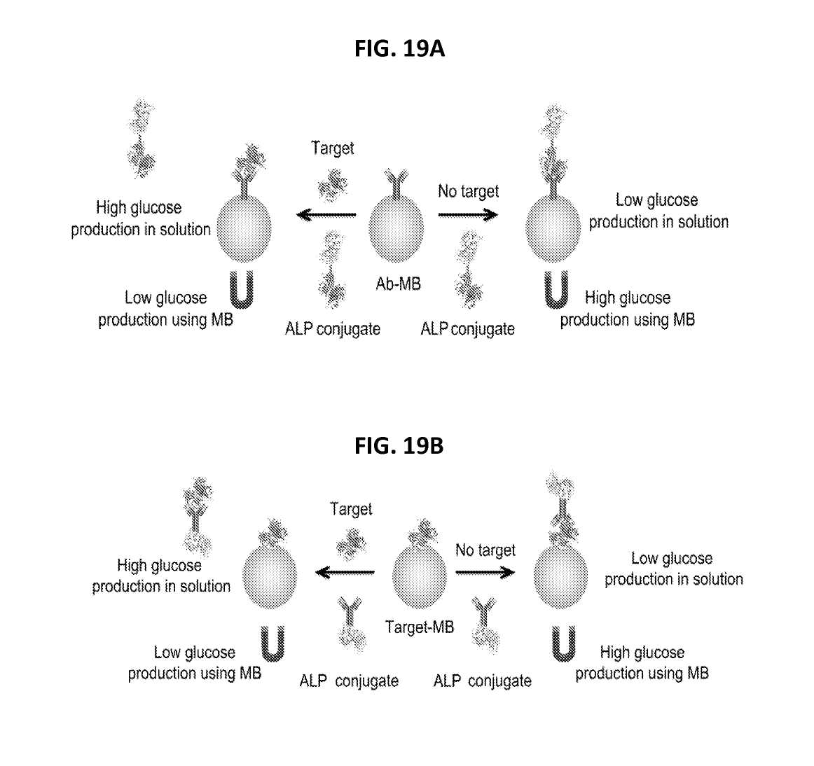

In one example of a competitive assay, the target competes with its ALP-target conjugate analogue to bind to a specific binding agent, such as an antibody or aptamer. The more targets in the sample, the less ALP-target conjugates are bound to the antibody and yield corresponding changes in the signal readout. For example as shown in FIG. 19A, antibody-coated magnetic beads (Ab-MBs) are mixed with alkaline-phosphate-target conjugate (ALP conjugate) and a sample which may or may not contain the target. One skilled in the art will appreciate that aptamer-coated beads can be used instead of or in addition to the Ab-MBs. If the target is present, it will compete for binding to the antibody with the ALP conjugate. If the target is absent, the ALP conjugate will bind to the antibody. After allowing the sample an ALP conjugate to interact with the Ab-MBs, the MBs are separated by a magnet. If the resulting solution is used (i.e., the solution resulting after MB removal) to produce glucose in the presence of glucose-1-phosphate, high and low glucose production occur for samples that contain or do not contain the target, respectively. This is a turn “on” version of the method because the more targets in the sample, the more glucose signal detected, and vice versa. In contrast, if the separated MBs are used for glucose production in glucose-1-phosphate solution, the situation reverses, thus providing a turn “off” method that gives lower glucose signals for the samples containing more targets and vice versa.

Another example is shown in FIG. 19B, where instead of using Ab-MB, target-coated magnetic bead (Target-MB) are used. In this case, Target-MBs re mixed with ALP-antibody conjugate (ALP conjugate) and a sample which may or may not contain the target. One skilled in the art will appreciate that ALP-aptamer conjugate can be used instead of or in addition to the ALP-antibody conjugate. The outcome of the test is the same as above: turn “on” if the solution phase is used to produce glucose, and turn “off” if the separated MBs are used for glucose production.

Thus, in one example of a competitive assay for detecting a target agent includes use of a target-specific binding agent-solid substrate complex, wherein the target-specific binding agent (e.g., antibody) specifically binds to the target. Examples of solid substrates include magnetic beads (MBs), such as amine-modified magnetic beads, as well as array plates (such as an ELISA plate). The test sample is incubated with the target-specific binding agent-solid substrate complex, and with an ALP-target conjugate (ALP conjugate), under conditions that allow the target (if present) to bind to the target-specific binding agent-solid substrate complex (to form a target-specific binding agent-solid substrate complex-target complex) or allow the ALP conjugate to bind to the target-specific binding agent-solid substrate complex (to form a target-specific binding agent-solid substrate complex-ALP conjugate complex) if the target is not present tin the sample. The solid substrate can be removed from the rest of the reaction, for example by removing solution from a well of a multi-well plate, using a blocker in a lateral flow device, or using a magnet to remove magnetic beads from solution. Thus, the target-specific binding agent-solid substrate complex-target agent complex or the target-specific binding agent-solid substrate complex-ALP conjugate complex can be separated from a solution in which the target-specific binding agent-solid substrate complex-target agent complex or target-specific binding agent-solid substrate complex-ALP conjugate complex is present. The resulting target-specific binding agent-solid substrate complex-target agent complex, target-specific binding agent-solid substrate complex-ALP conjugate complex, the solution which contained the target-specific binding agent-solid substrate complex-target agent complex or the solution which contained the target-specific binding agent-solid substrate complex-ALP conjugate complex are contacted with glucose-1-phosphate under conditions that permit ALP to convert the glucose-1-phosphate to glucose. The resulting glucose can be detected with a PGM and a determination made as to whether the target is present or absent in the sample by correlating the glucose detected. For example, if the target is present, there will be high glucose present in the solution that contained the target-specific binding agent-solid substrate complex-target agent complex, but not if the target-specific binding agent-solid substrate complex-target agent complex is used to detect glucose. In contrast, if the target is absent, there will be low or no glucose present in the solution that contained the target-specific binding agent-solid substrate complex-ALP conjugate complex but will be high if the target-specific binding agent-solid substrate complex-ALP conjugate complex is used to detect glucose.

Another example of a competitive assay for detecting a target agent includes use of a target-solid substrate complex. Examples of solid substrates include magnetic beads (MBs), such as amine-modified magnetic beads, as well as array plates (such as an ELISA plate). The target immobilized to the solid substrate can be the target itself, an analog of the target, or a portion of the target, such as an epitope. The test sample is incubated with the target-solid substrate complex, and with an ALP-target-specific antibody conjugate (ALP conjugate), under conditions that allow the target (if present) to bind to the ALP conjugate (to form a target-ALP conjugate complex) or allowing the ALP conjugate to bind to the target-solid substrate complex (to form a target-solid substrate-ALP conjugate complex) if the target is not present in the test sample. The solid substrate can be removed from the rest of the reaction, for example by removing solution from a well of a multi-well plate, using a blocker in a lateral flow device, or using a magnet to remove magnetic beads from solution. Thus, the target-solid substrate complex can be separated from a solution in which the target-ALP conjugate complex is present, and the target-solid substrate-ALP conjugate complex can be separated from a solution in which the target-solid substrate-ALP conjugate complex is present. The resulting target-solid substrate complex, solution containing the target-ALP conjugate complex, the target-solid substrate-ALP conjugate complex, or the solution which contained the target-solid substrate-ALP conjugate complex are contacted with glucose-1-phosphate under conditions that permit ALP to convert the glucose-1-phosphate to glucose. The resulting glucose can be detected with a PGM and a determination made as to whether the target is present or absent in the sample by correlating the glucose detected. For example, if the target is present, there will be high glucose present in the solution that contains the target-ALP conjugate complex, but not if the target-solid substrate complex is used to detect glucose. In contrast, if the target is absent, there will be low or no glucose present in the solution that contained the target-solid substrate-ALP conjugate complex but will be high if the target-solid substrate-ALP conjugate complex is used to detect glucose.

### Exemplary Sandwich Assays

In some examples, sandwich assay methods can be used to detect a target, such as a target of interest having numerous binding sites (e.g., some large molecular targets). Thus, in some examples a sandwich assay method is used to detect a multi-epitope target.

Thus, in some examples the method for detecting one or more target agents, includes contacting a test sample with a target-specific binding agent-solid substrate complex, wherein the target-specific binding agent (such as an antibody or aptamer) specifically binds to the target (for example at a first epitope). The first target-specific binding agent-solid substrate complex is incubated under conditions for allow it to bind to the target (if it is present in the sample), thereby forming a first target-specific binding agent-solid substrate complex-target complex (when the target is present in the test sample). The first target-specific binding agent-solid substrate complex-target agent complex is contacted with a second target-specific binding agent (such as an antibody or aptamer specific for a second epitope on the target), under conditions that permit formation of a first target-specific binding agent-solid substrate complex-target agent-second target-specific binding agent complex. The resulting complex is contacted with an ALP-labeled antibody under conditions that permit formation of a first target-specific binding agent-solid substrate complex-target agent-second target-specific binding agent-ALP-antibody complex. In some examples, the first target-specific binding agent-solid substrate complex-target agent-second target-specific binding agent-ALP-antibody complex is separated or removed from the rest of the reaction (such as a solution in which it is present), for example by removing solution from a well of a multi-well plate, using a blocker in a lateral flow device, or using a magnet to remove magnetic beads from solution. The first target-specific binding agent-solid substrate complex-target agent-second target-specific binding agent-ALP-antibody complex is contacted with glucose-1-phosphate under conditions that permit ALP to convert the glucose-1-phosphate to glucose. The resulting glucose can be detected with a PGM and a determination made as to whether the target is present or absent in the sample by correlating the glucose detected. For example, more glucose will be detected if the target is present in the sample than if the target is not present in the sample.

Exemplary Disorders that can be Diagnosed

The ability to detect a target agent, such as a target enzyme or protein, permits diagnosis of disorders. In some examples the methods include selecting a subject having or suspected of having a particular disease, such as those disorders described below and throughout the application. In some examples, the sample to be analyzed using the disclosed methods and sensors has or is suspected of having particular disease, such as those disorders described below and throughout the application.

Classic Galactosemia

Based on the ability of the disclosed methods to detect galactose-1-phosphate uridylyltransferase (GALT) activity, the disclosed methods can be used to diagnose classic galactosemia. Galactosemia is an inherited disorder in which the human body is unable to metabolize galactose. In one example, the disclosed sensors and methods can be used to screen newborns for classic galactosemia. Thus in some examples, the method includes selecting a newborn, such as a human infant. In some examples, the sample is obtained from a newborn, such as one less than 1 day old, less than 2 days old, less than 7 days old, less than 14 days old, or less than 30 days old, such as 24 hours to 7 days old. In one example, detection of significantly reduced levels of GALT activity or no GALT activity (for example relative to a normal control or reference value(s)) indicates that the patient from which the sample was obtained has classic galactosemia. For example, if GALT activity is reduced by at least 80%, at least 90%, at least 95%, or at least 99%, relative to GALT activity detected in a sample from a normal individual (such as one without classic galactosemia or normal levels of GALT), this would lead to a diagnosis of classic galactosemia in the test patient.

In some examples, the disclose methods provide quantitative results, and can be used to determine if the subject has galactosemia, is a carrier, or is normal.²⁷ for example, by quantitating the amount of GALT activity measured, one can distinguish between these three types of individuals. In some examples, a GALT activity level of greater than 3.5 u/gHb indicates a normal individual, a GALT activity level of 3.2-3.5 u/gHb indicates a carrier individual, and a GALT activity level of less than or equal to 3.1 u/gHb indicates a galactosemia individual,

Classic galactosemia, also called galactosemia type 1 or GALT deficiency, is the most common type of galactosemia caused by the deficiency of GALT enzyme due to the variants (mutations) in the GALT gene.^{19,20} It occurs at a rate about 1:23 000 to 1:44 000 in Western Europe and about 1:50 000 to 1:70 000 in USA, respectively.^{21,22} Feeding lactose- or galactose-containing food to patients with classic galactosemia results in the accumulation of high levels of galactose-1-phosphate in blood circulation, and causes vomiting, diarrhea, lethargy, jaundice, liver enlargement, cataracts, and even death.^{19,20} It is especially hazardous to newborns because the infants are immediately fed by their mothers' milk that contains high concentrations of lactose. Currently, there is still no cure available for classic galactosemia, so that the newborns or patients with classic galactosemia can only be treated with lactose- and galactose-free diet to prevent the serious consequences induced by lactose or galactose digestion. Therefore, the diagnosis of classic galactosemia is critical for the patients to avoid ingestion of lactose- or galactose-containing food and reduce or eliminate the risk of permanent health damage or death.

Currently, the diagnosis of galactosemia is routinely carried out in newborn screens.²⁰ The most frequently used clinic tests for screening classic galactosemia is the Beutler-Baluda fluorescent spot test that measures the GALT activity in blood using phosphoglucomutase and glucose-6-phosphate dehydrogenase,^{23,24} while other less common tests determine the concentrations of blood galactose and galactose-1-phosphate.²⁵ In the Beutler-Baluda test, GALT catalyzes the transfer of uridine phosphate from uridine diphosphate glucose (UDP-glucose) to galactose-1-phosphate to yield uridine diphosphate galactose (UDP-galactose) and glucose-1-phosphate, followed by phosphoglucomutase-catalyzed transformation of glucose-1-phosphate to glucose-6-phosphate, and subsequently glucose-6-phosphate is quantified by the fluorescence enhancement resulting from the reduction of nicotinamide adenine dinucleotide phosphate (NADP⁺) to its reduced form of NADPH by glucose-6-phosphate in the presence of glucose-6-phosphate dehydrogenase (G6PD). Finally, the rate of fluorescence change serves as a measurement of the GALT activity. In the Beutler-Baluda tests for screening classic galactosemia, quantitative information of the GALT activity can determine the status of classic galactosemia and determine if the patient is the carrier of the inherited metabolic defects compared to galactosemia or normal.

Although newborn screen tests are widely used in hospitals and medical centers, a simple, portable and quantitative GALT activity assay without the use of expensive laboratory-based instrument permits screening of classic galactosemia by the public at home or in the field (for example in rural areas). For example, such methods and sensors can be valuable in less developed rural areas and low-income countries, where the medical service is unavailable or not well established (e.g., in resource-limited locations). Although some colorimetric assays using the similar principles as the Beutler-Baluda tests can be utilized for the assessment of GALT activity via eye observation of color or fluorescence change without any instrument for potential public use,²⁶ they can only achieve qualitative or semi-quantitative detection unless laboratory-based spectrometers are used. Quantitative results allow for accurate differentiation between galactosemia, carrier, and normal individuals.²⁷ In addition, the intrinsic color of blood can interfere with colorimetric methods, thus requiring the removal of hemoglobin and other serum proteins. Thus, in some examples, the disclosed methods do not require separation of the sample prior to its analysis (for example a blood sample can be used directly without further separation).

Disorders Associated with Glycogen Phosphorylase

The ability to detect glycogen phosphorylase (GP) permits diagnosis of several disorders. For example, detection of the muscle isoform of GP (PYGM) permits diagnosis of McArdle disease, wherein decreased levels of PYGM indicates the presence of McArdle disease. Detection of the liver isoform of GP (PYGL) permits diagnosis of glycogen storage disease type VI, wherein decreased levels of PYGL indicates the presence of glycogen storage disease type VI. Detection of the brain isoform of GP (BGP) (for example in a tumor biopsy sample) permits diagnosis of gastric cancer or colorectal carcinoma, wherein increased levels of BGP indicates the presence of gastric cancer or colorectal carcinoma.

In one example, detection of significantly reduced levels of PYGM or PYGL activity or no PYGM or PYGL activity (for example relative to a normal control) indicates that the patient from which the sample was obtained has McArdle disease or glycogen storage disease type VI, respectively. For example, if PYGM or PYGL activity is reduced by at least 80%, at least 90%, at least 95%, or at least 99%, relative to PYGM or PYGL activity detected in a sample from a normal individual (such as one without McArdle disease or glycogen storage disease type VI, respectively or normal levels of PYGM or PYGL, respectively), this would lead to a diagnosis of McArdle disease or glycogen storage disease type VI, respectively in the test patient.

In one example, detection of significantly increased levels of BGP activity (for example relative to a normal control) indicates that the patient from which the sample was obtained has gastric cancer or colorectal carcinoma. For example, if BGP activity is increased by at least 20%, at least 50%, at least 100%, or at least 200%, relative to BGP activity detected in a sample from a corresponding normal sample (e.g., non-cancerous gastric or colorectal sample), this would lead to a diagnosis of gastric cancer or colorectal carcinoma in the test patient.

Disorders Associated with Galactokinase Deficiencies

Galactokinase converts galactose into galactose-1-phosphate, which can be converted to glucose-1-phosphate in the presence of UDP-glucose and GALT (GALT controls the equilibrium of UDP-glucose+galactose-1-phosphate into UDP-galactose+glucose-1-phosphate). Detection of galactokinase can be used to diagnose galactosemia and galactosemia type II (symptoms include early onset cataracts).

In one example, detection of significantly reduced levels of galactokinase activity or no galactokinase activity (for example relative to a normal control) indicates that the patient from which the sample was obtained has galactosemia or galactosemia type II. For example, if galactokinase activity is reduced by at least 80%, at least 90%, at least 95%, or at least 99%, relative to galactokinase activity detected in a sample from a normal individual (such as one without galactosemia or galactosemia type II or normal levels of galactokinase) this would lead to a diagnosis of galactosemia or galactosemia type II in the test patient.

Disorders Associated with UDP-Glucose 4-Epimerase Deficiencies

UDP-glucose 4-epimerase catalyzes the reversible conversion of UDP-galactose into UDP-glucose, which can then be used with galactose-1-phosphate for the production of glucose-1-phosphate by GALT. Detection of UDP-glucose 4-epimerase can be used to diagnose type III galactosemia.

In one example, detection of significantly reduced levels of UDP-glucose 4-epimerase activity or no UDP-glucose 4-epimerase activity (for example relative to a normal control) indicates that the patient from which the sample was obtained has galactosemia type III. For example, if UDP-glucose 4-epimerase activity is reduced by at least 80%, at least 90%, at least 95%, or at least 99%, relative to UDP-glucose 4-epimerase activity detected in a sample from a normal individual (such as one without galactosemia type III or normal levels of UDP-glucose 4-epimerase), this would lead to a diagnosis of galactosemia type III in the test patient.

Diabetes

Detection of HbA1c can be Used to Diagnose Diabetes.

In one example, detection of significantly increased levels of HbA1c (for example relative to a normal control) indicates that the patient from which the sample was obtained has diabetes. For example, if HbA1c activity is increased by at least 20%, at least 50%, at least 100%, or at least 200%, relative to HbA1c activity detected in a sample from a corresponding normal patient (e.g., one without diabetes), this would lead to a diagnosis of diabetes (such as diabetes mellitus) in the test patient. In one example, detection of levels of HbA1c≧48 mmol/mol (≧6.5%) indicates a diagnosis of diabetes.

Other Diseases

The ability to detect proteins using the disclosed methods permits diagnosis of many disorders. Exemplary diseases include, but are not limited to: (1) cardiovascular disorders by detection of one or more of: cardiac troponins (TnI and TnT), myoglobin, d-dimer, c-reactive protein, liproprotein PLA2, myeloperoxidase, ischemia modified albumin, pregnancy-associated plasma protein-A, placenta-derived growth factor, and soluable CD40 ligand (sCD40L); (2) heart failure by detection of one or more of: brain natriuretic peptide (BNP), NT-proBNP, mid-regional pro-atrial natriuretic peptide (MR-proANP), and mid-regional pro-adrenomedullin (MR-proADM); (3) diabetes by detection of one or more of: HbA1c, albumin, fructosamine, islet cell antibodies, insulin autoantibody, autoantibody to the 65 kDa isoform of glutamic acid decarboxylase, and autoantibody to tyrosine phosphatase related IA-2 molecule; (4) cancer by detection of one or more of: prostate cancer antigen, alpha-fetoprotein, beta-hCG, beta-2-microglobulin, carcinoembryonic antigen, CD20 and, fibrin/fibrinogen; (5) gut diseases by detection of one or more of: fecal calprotein, lactoferrin, fecal serine protease, and citrulline; (6) rheumatoid arthritis by detection of: anti-CCP; and (7) Alzheimer's disease by detection of one or more of: amyloid beta protein and tau.

### Samples

Any biological or environmental specimen that may contain (or is known to contain or is suspected of containing) a target agent can be used. Biological samples are usually obtained from a subject and can include genomic DNA, RNA (including mRNA), protein, or combinations thereof. Examples include a tissue or tumor biopsy, fine needle aspirate, bronchoalveolar lavage, pleural fluid, spinal fluid, saliva, sputum, surgical specimen, lymph node fluid, ascites fluid, peripheral blood or fractions thereof (such as serum or plasma), urine, saliva, buccal swab, and autopsy material. Techniques for acquisition of such samples are well known in the art. Serum or other blood fractions can be prepared in the conventional manner. Samples can also include fermentation fluid and tissue culture fluid.

Environmental samples include those obtained from an environmental media, such as water, air, soil, dust, wood, plants or food.

In other examples, a sample includes a control sample, such as a sample known to contain or not contain a particular amount of the target agent.

In one example the sample is a food sample, such as a meat, fruit, or vegetable sample. For example, using the methods provided herein, adulterants in food products can be detected, such as a pathogen or toxin or other harmful product.

Once a sample has been obtained, the sample can be used directly, concentrated (for example by centrifugation or filtration), purified, liquefied, diluted in a fluid, or combinations thereof. In some examples, the sample is not manipulated prior to its analysis, other than to apply it to the sensor. For example, in some examples the sample is not pre-treated to remove proteins (e.g., hemoglobin or other serum proteins) prior to analysis of the sample. In some examples, proteins or pathogens are extracted from the sample, and the resulting preparation analyzed using the methods provided herein.

### Sensors for Detecting Target Agents

Provided herein are sensors that can be used to detect one or more target agents or target agent activities. Such sensors can be engineered using the methods provided herein to detect a broad range of targets, significantly facilitating rational design and increasing the efficiency of sensor development. Such sensors take advantage of the ability of ALP to convert glucose-1-phosphate to glucose, which can be detected using a glucose meter, such as a PGM. In some examples, one or more sensors are part of a lateral flow device. In some examples, one or more sensors are part of a microfluidic device. In some examples, multiple sensors are combined, thereby permitting detection of more than one target. Using this general methodology, sensitive and selective particular examples of sensors for the quantification of GALK, GP, and HbA1c are reported herein that require only a commercially available PGM to do the detections. Using this platform, many other sensors for various targets using a PGM can be achieved through the general approach described herein.

In one example, the sensor is based on a sandwich type assay, such as shown in FIG. 12. In one example, such a sensor includes a solid support to which is attached a specific binding agent (such as an antibody or aptamer specific for a first epitope) that specifically binds to the target agent in the presence of the target agent but not significantly to other agents. The sensor also includes a second specific binding agent (such as an antibody or aptamer specific for a second epitope) that specifically binds to the target agent, and is labeled with ALP, for example by using a labeled ALP antibody, which can bind to the second specific binding agent.

Other examples of sensors use competitive binding, such as shown in FIGS. 19A and 19B. In one example, such a sensor includes a solid support to which is attached a specific binding agent (such as an antibody or aptamer) that specifically binds to the target agent in the presence of the target agent but not significantly to other agents. The sensor also includes an alkaline-phosphate-target conjugate (ALP conjugate), which can compete with the target for binding to the specific binding agent on the solid support. In another example, the sensor includes a solid support to which is attached the target. The sensor also includes an ALP-specific binding agent conjugate (ALP conjugate), wherein the specific binding agent (such as an antibody or aptamer) that specifically binds to the target agent in the presence of the target agent but not significantly to other agents. The target in the sample (if present) will compete with the target on the solid substrate for binding to the specific binding agent on the ALP-specific binding agent conjugate.

Sensors for Detecting Target Agents that Produce Glucose-1-Phosphate

Disclosed herein are sensors that permit detection of one or more target agents, such as an enzyme, that can convert one or more starting products to glucose-1-phosphate, directly or as an early step in a pathway to produce glucose-1-phosphate. If the target enzyme produces glucose-1-phosphate directly from a starting product (such as galactose-1-phosphate), additional enzymes may not be required. However, if the target enzyme does not produce glucose-1-phosphate directly, but instead produces a product in the pathway that can be converted to glucose-1-phosphate by a second enzyme, the second enzyme (or more enzymes as needed) can be supplied (for example with the starting products).

In one example, the sensor includes a solid support to which is attached one or more starting products (wherein at least one of the starting products can be converted to glucose-1-phosphate by a target enzyme, comprises an enzyme involved in the production of glucose-1-phosphate, or combinations thereof), and ALP, which results in glucose production. The glucose can be detected and in some examples quantified, using a PGM. In some examples, the amount of glucose produced is proportional to the amount of target agent or activity in the test sample. Thus if glucose is detected, this indicates that the target enzyme or enzyme activity is present in the sample. If glucose is not detected, this indicates that the target enzyme or enzyme activity is not present in the sample.

In another example, the sensor includes a solid support to which includes one or more discrete regions having attached thereto different desired reagents, and one or more mixing chambers were desired reactions can occur. The solid support can also have an entry and exit port. In one example, the solid support includes a region that includes buffer reagents, another regions has one or more starting products (wherein at least one of the starting products can be converted to glucose-1-phosphate by a target enzyme, comprises an enzyme involved in the production of glucose-1-phosphate, or combinations thereof), a region that includes ALP and suitable buffers, and optionally a region that includes agents that can neutralize a basic pH to a neutral pH, such as a phosphate buffer (e.g., sodium, potassium or ammonium salts, for example, NaH₂PO₄ and the like), as well as other buffers such as Tris-HCl, HEPES, MES, NaHCO₃—Na₂CO₃, and the like. The device can also include a first mixing chamber between the region containing one or more starting products and the region containing ALP, and a second mixing chamber between the region containing ALP and the pH neutralizing agents. The glucose can be released from the device, for example thru an exit port, wherein it is subsequently detected and in some examples quantified, using a PGM. In some examples, the amount of glucose produced is proportional to the amount of target agent or activity in the test sample. Thus if glucose is detected, this indicates that the target enzyme or enzyme activity is present in the sample. If glucose is not detected, this indicates that the target enzyme or enzyme activity is not present in the sample.

In one example, the target enzyme is GALT. In such an example, the one or more starting products include UDP-glucose and galactose-1-phosphate. Thus, the solid support includes an area or region that has attached thereto UDP-glucose and galactose-1-phosphate. A different area of the solid support has attached thereto ALP.

In another example, the target enzyme is GP. In such an example, the one or more starting products include glycogen and 5′-AMP. Thus, the solid support includes an area or region that has attached thereto glycogen and 5′-AMP. A different area of the solid support has attached thereto ALP.

In one example, the target enzyme is GALK. In such an example, the one or more starting products include α-D-galactose, UDP-glucose, and GALT (a non-target enzyme in this example). Thus, the solid support includes an area or region that has attached thereto α-D-galactose, UDP-glucose, and GALT. A different area of the solid support has attached thereto ALP.

In another example, the target enzyme is UDP-glucose 4-epimerase. In such an example, the one or more starting products include UDP-galactose, galactose-1-phosphate and GALT. Thus, the solid support includes an area or region that has attached thereto DP-galactose, galactose-1-phosphate and GALT. A different area of the solid support has attached thereto ALP.

The solid support can further include attached thereto agents that permit detection of glucose, such as materials routinely found on commercially available glucose meter strips. For example, the “endpoint” of the disclosed solid supports (for example an area containing glucose or from where glucose is release) can be connected to the start point of any commercial glucose meter strip (such as those containing glucose oxidase). Exemplary glucose meter strips include but are not limited to Glucocard® and OneTouch® (and those described in U.S. Pat. Nos. 6,413,410 and 6,733,655). Thus, in one example, the solid support further includes a region that has attached thereto glucose oxidase.

In some examples, the solid support includes a region that has attached thereto reagents that can neutralize a basic pH to a neutral pH, such as a phosphate buffer (e.g., sodium, potassium or ammonium salts, for example, NaH₂PO₄ and the like), as well as other buffers such as Tris-HCl, HEPES, MES, NaHCO₃—Na₂CO₃, and the like.

The sensor can include additional materials, such as one or more reaction pads (or membranes) on which the desired reactions can occur, a sample pad to which the sample is applied, an absorption pad where the produce glucose is delivered and read by a glucose meter, or combinations thereof.

Methods of using the sensors (such as a lateral flow device or microfluidic) for one or more target agents are also provided. For example, such a method can include contacting a sensor with a test sample under conditions sufficient to allow a target in the test sample to interact with the one or more starting products, under conditions wherein glucose-1-phosphate is produced. In some examples, this reaction occurs on a first reaction pad. The method can further include allowing the glucose-1-phosphate to interact with the ALP on the sensor under conditions wherein glucose will form. In some examples, this reaction occurs on a second reaction pad. The resulting glucose is detected, for example with a PGM. In some examples, the glucose is present on an absorption pad, which is read by a PGM. The method includes determining that the target agent is present in the sample when glucose is detected.

In a specific example, the method includes contacting one or more lateral flow devices with a sample under conditions sufficient to allow the target agent in the sample to flow through the lateral flow device and interact with the one or more starting products present on the lateral flow device (for example on a first reagent pad) and generating glucose-1-phosphate on a first reaction pad present on the lateral flow device. The resulting glucose-1-phosphate is allowed to flow through the lateral flow device and interact with the ALP present on the lateral flow device (for example on a second reagent pad), thereby generating glucose on a second reaction pad present on the lateral flow device. In some examples, the glucose flows through the lateral flow device and interacts with pH neutralizing agents present on the lateral flow device. The resulting glucose is detected, for example with a PGM. In some examples, the glucose is present on an absorption pad, which is read by a PGM. The method includes determining that the target agent is present in the sample when glucose is detected.

Sensors for Detecting Targets that can Specifically Bind to an Antibody or Aptamer

Disclosed herein are sensors that permit detection of one or more target agents, such as targets that can bind with high specificity to a particular antibody or aptamer (such as a DNA aptamer). Any target agent for which there are antibodies or aptamers, or for which antibodies or aptamers³³ can be generated, can be quantified by a glucose meter using the sensors and methods provided herein. In some examples, more than one antibody and/or aptamer specific for the target is used. For example, two different antibodies may bind to different epitopes of the same target. Aptamers are nucleic acids (such as DNA or RNA) that recognize targets with high affinity and specificity. Methods of making aptamers are known (e.g., see Liu et al., Proc. Nat. Acad. Sci. U.S.A. 2007, 104, 2056-2061). Although “antibody” is commonly used herein, one will recognize that an aptamer can be used instead.

In one example, for example with a sandwich-type assay, the sensor includes a solid support which includes a first target-specific binding agent (e.g., antibody or aptamer) and a second target-specific binding agent (e.g., antibody or aptamer), wherein the first and the second target-specific binding agents bind to different epitopes of the target agent. In one example, either the first or the second target-specific binding agent is immobilized to an area of the solid support. The other target-specific binding agent is not immobilized and is labeled with ALP (for example directly or with an ALP-labeled secondary antibody), but merely attached such that it can be mobilized (for example during flow thru a lateral flow strip or microfluidic device). Methods of immobilizing antibodies or nucleic acid aptamers to a solid support are well known in the art. In some examples, the first and second target-specific binding agents are antibodies, such as polyclonal antibodies, monoclonal antibodies, or fragments thereof. In one example, the first antibody is a polyclonal antibody or fragments thereof, and the second antibody is a monoclonal antibody, or fragment thereof. In another example, the first antibody is a monoclonal antibody or fragment thereof, and the second antibody is a polyclonal antibody, or fragment thereof. The first and second target-specific binding agents are arranged on the support such that each can interact with the target at the desired time.

The solid support of the sensor can further have attached thereto glucose-1-phosphate. The glucose-1-phosphate and the first and second target-specific binding agents are arranged on the support such that the ALP-labeled target-specific binding agent that does not bind to the immobilized target-specific binding agent can interact with the glucose-1-phosphate at the desired time, for example to allow glucose production.

The first target-specific binding agent, second target-specific binding agent, and glucose-1-phosphate are all attached to different areas of the solid support, but in some examples, only the first or second target-specific binding agent (e.g., antibody or aptamer) is immobilized, such that it cannot be moved by the flow of a lateral flow or microfluidic device. As a result, the complex containing the target, first and second target-specific binding agents (one of which is labeled with ALP) remains immobilized, and any ALP-target-specific binding agent that does not bind to the target is a part of this immobilized complex is not available to react with other agents, such as glucose-1-phosphate. Materials in the mobile phase, such as ALP-target-specific binding agent that does not bind to the target, are contacted with glucose-1-phosphate under conditions wherein ALP can convert the glucose-1-phosphate to glucose. The resulting glucose is detected with a glucose meter, such as a PGM. The more target in the sample, the less of the ALP there is to interact with the glucose-1-phosphate, therefore giving less glucose signal. The target agent is detected in the sample by correlating the glucose detected wherein an amount of glucose detected inversely corresponds to an amount of target enzyme in the sample. Thus if glucose is detected, this indicates that the target is not present in the sample. If glucose is not detected, this indicates that the target is present in the sample.

In another example, the sensor includes a solid support having separate areas. The solid support can include a first area having attached thereto a first functional molecule-conjugated antibody specific for a target agent, an agent that binds to the functional molecule (such as avidin, streptavidin or neutravidin if the functional molecule is biotin or a fluorescein antibody if the functional molecule is fluorescein), and functional molecule conjugated to ALP. In some examples the functional molecule—first antibody, agent that binds to the functional molecule, and functional molecule ALP, are present on the solid support as a first complex (functional molecule—first antibody—agent that binds to the functional molecule—functional molecule—ALP). This first complex is attached such that it can be mobilized by the flow in a lateral flow or microfluidic device. The solid support can further include a second area having immobilized thereto (such that it and agents bound to it do not move with the flow in a lateral flow or microfluidic device) a second antibody specific for the target agent. The first antibody and second antibody (or aptamers) bind to different epitopes of the target agent. The solid support can further include a third area having attached thereto glucose-1-phosphate, such glucose-1-phosphate can be mobilized by the flow in a lateral flow or microfluidic device. As a result, the complex containing the first complex, target, second antibody and ALP-secondary antibody remains immobilized, and the ALP that is a part of this complex is not available to react with other agents, such as glucose-1-phosphate. Molecules not immobilized to the solid substrate, such as functional molecule—ALP that did not bind the complex, are contacted with glucose-1-phosphate under conditions wherein ALP can convert the glucose-1-phosphate to glucose. The resulting glucose is detected with a glucose meter, such as a PGM. The more target in the sample, the less of the ALP there is to interact with the glucose-1-phosphate, therefore giving less glucose signal. The target agent is detected in the sample by correlating the glucose detected wherein an amount of glucose detected inversely corresponds to an amount of target enzyme in the sample. Thus if glucose is detected, this indicates that the target is not present in the sample. If glucose is not detected, this indicates that the target is present in the sample.

In one example, for example with a competitive-type assay, the sensor includes a solid support which includes a target-specific binding agent (e.g., antibody or aptamer). The target-specific binding agent (e.g., antibody or aptamer) is immobilized to an area of the solid support. Methods of immobilizing antibodies or nucleic acid aptamers to a solid support are well known in the art. In some examples, the antibody is a polyclonal antibody, monoclonal antibody, or fragment thereof. The sensor also includes a complex containing ALP and the target (an ALP conjugate), which can compete with the target for binding to the target-specific binding agent on the solid support. The target-specific binding agent-solid support and ALP conjugate are arranged on the support such that each can interact with the target at the desired time.

In another example of a competitive-type assay the sensor includes a solid support to which the target is immobilized. The target can be the target or an analog thereof, or a fragment or portion thereof. Methods of immobilizing reagents to a solid support are well known in the art. The sensor also includes a complex containing ALP and a target-specific binding agent (ALP conjugate). The target-specific binding agent (such as an antibody or aptamer) specifically binds to the target agent in the presence of the target agent but not significantly to other agents. The target in the sample (if present) will compete with the target on the solid substrate for binding to the target-specific binding agent on the ALP-target-specific binding agent conjugate. The target-solid support and ALP conjugate are arranged on the support such that each can interact with the target at the desired time.

The solid support of the competitive assay sensors can further have attached thereto glucose-1-phosphate. The glucose-1-phosphate and the target-specific binding agent-solid support or target-solid support and the ALP conjugate are arranged on the support such that the ALP can interact with the glucose-1-phosphate at the desired time, for example to allow glucose production.

The target-specific binding agent-solid support or target-solid support, the appropriate ALP conjugate (e.g., see FIGS. 19A and 19B), and glucose-1-phosphate are all attached to different areas of the solid support. In one example, the target-specific binding agent-solid support or target-solid support is immobilized, such that it cannot be moved by the flow of a lateral flow or microfluidic device. As a result, target-specific binding agent-solid support bound to the target, or target-solid support, remains immobilized and is not available to react with other agents, such as glucose-1-phosphate. However, materials in the mobile phase, such the ALP complex containing the target, or the ALP-target-specific binding agent conjugate bound to the target are contacted with glucose-1-phosphate under conditions wherein ALP can convert the glucose-1-phosphate to glucose. The resulting glucose is detected with a glucose meter, such as a PGM. The more target in the sample, the more of the ALP there is to interact with the glucose-1-phosphate, therefore giving more glucose signal. The target agent is detected in the sample by correlating the glucose detected wherein an amount of glucose detected corresponds to an amount of target enzyme in the sample. Thus if glucose is detected, this indicates that the target is present in the sample. If glucose is not detected, this indicates that the target is absent in the sample.

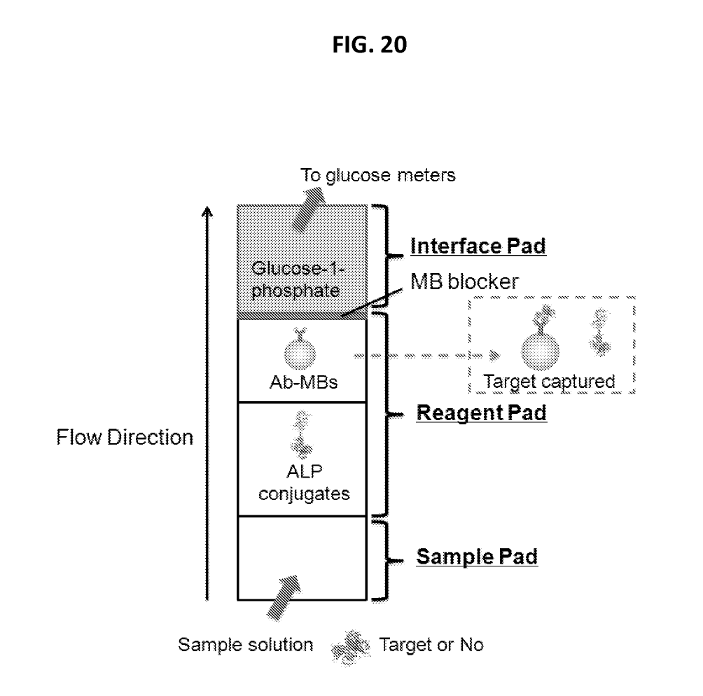

For example, as shown in FIG. 20, samples containing target or not are applied to the sample pad of a lateral flow device. The solution goes from the bottom to top by capillary force. In the lower reagent pad, the solution takes ALP conjugates along with it, and then they reach the higher reagent pad preload with Ab-MBs (or target-MBs) (one skilled in the art will appreciate that the MBs can be other solid supports and that the Ab can be an aptamer). At that position, target in the sample, ALP conjugates and Ab-MBs (or target-MBs) initiate the competitive binding. In the presence of target, targets will bind on Ab-MBs (as shown in the dash-lined rectangle), thus allowing ALP conjugates to reach interface pad to produce glucose from glucose-1-phosphate for glucose meter measurement. In contrast, if no target is present in the sample, ALP conjugates will be captured by Ab-MBs and cannot reach the interface pad, resulting in no/low glucose production. Therefore, the more target in the sample, the higher glucose signal will be detected in the glucose meter.

However, if instead the target-specific binding agent-solid support or target-solid support is in the mobile phase, and the ALP complex containing the target, or the ALP-target-specific binding agent conjugate bound to the target are in the immobile phase, then the target-specific binding agent-solid support or target-solid support are contacted with glucose-1-phosphate under conditions wherein ALP can convert the glucose-1-phosphate to glucose. The resulting glucose is detected with a glucose meter, such as a PGM. The more target in the sample, the less of the ALP there is to interact with the glucose-1-phosphate, therefore giving less glucose signal. The target agent is detected in the sample by correlating the glucose detected wherein an amount of glucose detected inversely corresponds to an amount of target enzyme in the sample. Thus if glucose is detected, this indicates that the target is absent from the sample. If glucose is not detected, this indicates that the target is present in the sample.

In one example, the areas of the sensor having reagents attached thereto are membranes, which can in some examples be interspersed with other solid supports without reagents (such as other membranes).

The solid support can further include attached thereto agents that permit detection of glucose, such as materials routinely found on commercially available glucose meter strips (such as glucose oxidase). In one example, the solid support can further include a region that has attached thereto glucose oxidase. For example, the “endpoint” of the disclosed solid supports (for example an area containing glucose) can be connected to the start point of any commercial glucose meter strip (such as those containing glucose oxidase). Exemplary glucose meter strips include but are not limited to Glucocard® and OneTouch® (and those described in U.S. Pat. Nos. 6,413,410 and 6,733,655).

In some examples, the solid support includes a region that has attached thereto reagents that can neutralize a basic pH to a neutral pH, such as a phosphate buffer (e.g., sodium, potassium or ammonium salts, for example, NaH₂PO₄ and the like), as well as other buffers such as Tris-HCl, HEPES, MES, NaHCO₃—Na₂CO₃, and the like.

The sensor can include additional materials, such as one or more reaction pads (or membranes) on which the desired reactions can occur, a sample pad to which the sample is applied, an absorption pad where the produce glucose is delivered and read by a glucose meter, or combinations thereof.

Methods of using the sensors (such as a lateral flow device) for one or more target agents are also provided. In one example, such a method can include contacting a sensor with a test sample under conditions sufficient to allow a target in the test sample to interact with an ALP-labeled first antibody specific for the target agent (for example one present on a first reagent pad), under conditions wherein an ALP-first antibody-target complex will form (for example on a first reaction pad). The resulting ALP-first antibody-target complex is contacted with the second antibody (for example one immobilized to on a second reagent pad) under conditions sufficient to allow the second antibody to specifically bind to the target agent in the ALP-antibody-target complex and under conditions wherein an ALP-first antibody-target-second antibody complex will form (for example on a second reaction pad). The ALP-first antibody-target-second antibody complex is immobilized, and thus is not present in the mobile phase. Only the mobile phase proceeds to a region on the sensor containing glucose-1-phosphate (for example on a third reagent pad) under conditions sufficient to allow the glucose-1-phosphate to interact with any ALP in the mobile phase under conditions wherein glucose will form (for example on a third reaction pad). The resulting glucose is detected, for example with a PGM. In some examples, the glucose is present on an absorption pad, which is read by a PGM. The method includes determining that the target agent is present in the sample when glucose is not detected or that that the target agent is absent in the sample when glucose is detected.

In a specific example, the method includes contacting one or more lateral flow devices with a sample under conditions sufficient to allow target agent in the sample to flow through the lateral flow device and interact with the ALP-first antibody specific for the target agent present on a first reagent pad of lateral flow device under conditions sufficient to allow a complex to form between the target agent and the first antibody (for example on a first reaction pad). The target agent-ALP-first antibody complex flows through the lateral flow device and interacts with the second antibody present on a second reagent pad of the lateral flow device under conditions sufficient to allow the second antibody to specifically bind to the target agent-ALP-first antibody complex, thereby generating a second antibody-target-ALP-first antibody complex (for example on a second reaction pad). The resulting a second antibody-target-ALP-first antibody is immobilized, and thus not in the mobile phase. Materials in the mobile phase (such as ALP-first antibody that did not bind to the target) are allowed to flow through the lateral flow device and interact with glucose-1-phosphate present on a third reagent pad of the lateral flow device under conditions sufficient to allow the glucose-1-phosphate to interact with the ALP in the mobile phase, thereby forming glucose (for example on a third reaction pad present on the lateral flow device). In some examples, the glucose flows through the lateral flow device and interacts with pH neutralizing agents present on the lateral flow device. The resulting glucose is detected, for example with a PGM. In some examples, the glucose is present on an absorption pad, which is read by a PGM. The method includes determining that the target agent is present in the sample when glucose is not detected or that that the target agent is absent in the sample when glucose is detected.

In another example, such a method can include contacting a sensor with a test sample under conditions sufficient to allow a target in the test sample to interact with the first antibody specific for the target agent, which can be present as part of a first complex (functional molecule—first antibody—agent that binds to the functional molecule—functional molecule—ALP) thereby forming a functional molecule—first antibody-target-agent that binds to the functional molecule—functional molecule—ALP complex (second complex) (for example on a reaction pad). The resulting second complex is contacted with the second antibody under conditions sufficient to allow the second antibody to specifically bind to the target agent in the second complex under conditions wherein functional molecule—first antibody-target-agent that binds to the functional molecule—functional molecule—ALP-second antibody complex (third complex) will form. The second target antibody is immobilized, thus preventing it and agents bound to it (such as the second complex) to be moved by flow (such as present in a lateral flow device or microfluidic device). The resulting third complex is immobilized, and thus the ALP that is a part of this complex is not available to react with other agents on later parts of the device, such as glucose-1-phosphate. Molecules not immobilized to the solid substrate, such as ALP of the first complex that did not bind the immobilized second antibody, are contacted with glucose-1-phosphate under conditions wherein ALP can convert the glucose-1-phosphate to glucose (for example on a reaction pad). The resulting glucose (for example on an absorption pad) is detected with a glucose meter, such as a PGM. The more target in the sample, the less of the ALP there is to interact with the glucose-1-phosphate, therefore giving less glucose signal. The target agent is detected in the sample by correlating the glucose detected wherein an amount of glucose detected inversely corresponds to an amount of target enzyme in the sample. Thus if glucose is detected, this indicates that the target is not present in the sample. If glucose is not detected, this indicates that the target is present in the sample.

In one example, for example with a competitive assay, such a method can include contacting a sensor with a test sample (for example on a sample pad) under conditions sufficient to allow a target in the test sample to bind to the target-specific binding agent immobilized on the solid support or bind to the target-specific binding agent that is part of the ALP-target-specific binding agent conjugate, thereby forming a target-specific binding agent-solid support-target complex or forming an ALP-target-specific binding agent-target complex, respectively, (for example on a reagent pad). If target is not present, the ALP-target conjugate will bind to the target-specific binding agent-solid support instead of the target, or the ALP-target-specific binding agent conjugate will bind to the target-solid support, thereby forming a target-specific binding agent-solid support-ALP-target conjugate complex or forming a target-solid support ALP-target-specific binding agent conjugate complex, respectively, (for example on a reagent pad). If the solid supports (e.g., beads) are immobilized (for example by a blocker), this prevents it and agents bound to it (such as the target or ALP-target-specific binding agent conjugate) to be moved by flow (such as present in a lateral flow device or microfluidic device). In contrast, the materials in the mobile phase (e.g., ALP-target-specific binding agent-target complex or the ALP-target complex) are contacted with glucose-1-phosphate under conditions wherein ALP can convert the glucose-1-phosphate to glucose (for example on an interface or reaction pad). The resulting glucose (for example on an absorption pad) is detected with a glucose meter, such as a PGM. The more target in the sample, the more ALP there is to interact with the glucose-1-phosphate, therefore giving more glucose signal. The target agent is detected in the sample by correlating the glucose detected wherein an amount of glucose detected corresponds to an amount of target enzyme in the sample. Thus if glucose is detected, this indicates that the target is present in the sample. If glucose is not detected, this indicates that the target is not present in the sample. One skilled in the art will appreciate that instead of the solid support being immobilized, the ALP conjugates (ALP-target conjugate or ALP-antibody conjugate) can be immobilized, and thus instead the solid supports (e.g., beads and materials bound thereto are detected), in which case the amount of glucose detected inversely corresponds to an amount of target enzyme in the sample.

In a specific example, the method includes contacting one or more lateral flow devices with a sample under conditions sufficient to allow target agent in the sample to flow through the lateral flow device and interact with the first antibody specific for the target agent present on a first reagent pad on the lateral flow device, and the agent that binds to the functional molecule to bind to the functional molecule on the first antibody and conjugated to the ALP under conditions wherein a functional molecule—first antibody-target agent—the agent that binds to the functional molecule—functional molecule—ALP complex (second complex) will form, wherein the streptavidin and biotin conjugated ALP are present on the first reagent pad. In some examples, a first complex (functional molecule—first antibody—agent that binds to the functional molecule—functional molecule—ALP) is present on the lateral flow device, and the target agent is allowed to interact with this complex to form the second complex.

The resulting second complex flows through the lateral flow device and interacts with the second antibody immobilized on a second reagent pad, under conditions sufficient to allow the second antibody to specifically bind to the target agent in the second complex, forming a third complex and thus immobilize the resulting third complex. The third complex is a functional molecule first antibody-target agent—agent that binds to the functional molecule—functional molecule—ALP-second antibody complex. The resulting third complex cannot flows through the lateral flow device. Instead, molecules not immobilized to the solid substrate, such as ALP of the first complex that did not bind the immobilized second antibody and thus are in the mobile phase, interact with the glucose-1-phosphate present on an reagent pad of the lateral flow device under conditions sufficient to allow the glucose-1-phosphate to interact with ALP under conditions wherein glucose will form (for example on a reaction pad). In some examples the method includes optionally allowing the glucose to flow through the lateral flow device and interact with pH neutralizing agents present on the lateral flow device (for example on an reagent pad including an acidic membrane with pH neutralizing agents). In some examples, the glucose flows to the absorption pad present on the lateral flow device, which is read by a PGM. The more target in the sample, the less of the ALP there is to interact with the glucose-1-phosphate, therefore giving less glucose signal. The target agent is detected in the sample by correlating the glucose detected wherein an amount of glucose detected inversely corresponds to an amount of target enzyme in the sample. Thus if glucose is detected, this indicates that the target is not present in the sample. If glucose is not detected, this indicates that the target is present in the sample.

In a specific example, such as with a competitive assay, the method includes contacting one or more lateral flow devices with a sample under conditions sufficient to allow target agent in the sample to flow through the lateral flow device and into a pad containing ALP conjugates (ALP-target conjugates or ALP-target-specific binding agent conjugates). The sample solution takes the ALP conjugates along with it, to a subsequent pad containing target-specific binding agent-solid supports (e.g., MBs having target-specific antibodies or aptamers thereon) or target-solid supports (e.g., MBs having immobilized thereon target). In the presence of target, targets will bind to the target-specific binding agent-solid supports or to the ALP-target-specific binding agent conjugates, thus allowing ALP to reach a subsequent pad to produce glucose from glucose-1-phosphate for glucose meter measurement. If no target is present in the sample, the ALP-target conjugates will be captured by target-specific binding agent-solid supports and cannot reach a subsequent pad containing glucose-1-phosphase, resulting in no/low glucose production. Alternatively, if no target is present in the sample, the ALP-target-specific binding agent conjugates will be captured by target-solid supports and cannot reach a subsequent pad containing glucose-1-phosphase, resulting in no/low glucose production. One skilled in the art will appreciate that the opposite result can be achieved if instead of the solid supports (e.g., MBs) being immobilized such that they cannot reach the glucose-1-phosphate containing pad, the ALP conjugates are immobilized or otherwise prevented from reaching the glucose-1-phosphate containing pad, in which case the amount of glucose detected will be inversely proportional to the amount of target in the sample.

### Solid Supports

The solid support which forms the foundation of the sensor can be formed from known materials, such as any water immiscible material. In some examples, suitable characteristics of the material that can be used to form the solid support surface include: being amenable to surface activation such that upon activation, the surface of the support is capable of covalently attaching desired reagents, such as starting products, ALP, antibodies, and the like; being chemically inert such that at the areas on the support not occupied by the desired reagents can interact with the target agent with desired specificity are not amenable to non-specific binding, or when non-specific binding occurs, such materials can be readily removed from the surface without removing the desired reagents or target agent.

A solid phase can be chosen for its intrinsic ability to attract and immobilize an agent, such as starting products, ALP, antibodies, and the like. Alternatively, the solid phase can possess a factor that has the ability to attract and immobilize an agent, such as a protein or antibody. The factor can include a charged substance that is oppositely charged with respect to, for example, the desired reagent or to a charged substance conjugated to the desired reagent. In another embodiment, a specific binding member may be immobilized upon the solid phase to immobilize its binding partner (e.g., an antibody). In this example, therefore, the specific binding member enables the indirect binding of the antibody to a solid phase material.

The surface of a solid support may be activated by chemical processes that cause covalent linkage of a desired reagent (e.g., ALP, glucose-1-phosphate, antibody specific for the target agent, beads or other supports having attached thereto antibodies, targets, or aptamers, or combinations thereof) to the support. However, any other suitable method may be used for immobilizing a desired reagent to a solid support including, without limitation, ionic interactions, hydrophobic interactions, covalent interactions and the like. The particular forces that result in immobilization of a desired reagent on a solid phase are not important for the methods and devices described herein.

In one example the solid support is a particle, such as a bead. Such particles can be composed of metal (e.g., gold, silver, platinum), metal compound particles (e.g., zinc oxide, zinc sulfide, copper sulfide, cadmium sulfide), non-metal compound (e.g., silica or a polymer), as well as magnetic particles (e.g., iron oxide, manganese oxide). In some examples the bead is a latex or glass bead. The size of the bead is not critical; exemplary sizes include 5 nm to 5000 nm in diameter. In one example such particles are about 1 μm in diameter.

In another example, the solid support is a bulk material, such as a paper, membrane, porous material, water immiscible gel, water immiscible ionic liquid, water immiscible polymer (such as an organic polymer), and the like. For example, the solid support can comprises a membrane, such as a semi-porous membrane that allows some materials to pass while others are trapped. In one example the membrane comprises nitrocellulose. In a specific example the solid support is part of a lateral flow device that includes one or more regions containing the sensors disclosed herein.

In some embodiments, porous solid supports, such as nitrocellulose, are in the form of sheets or strips, such as those found in a lateral flow device. The thickness of such sheets or strips may vary within wide limits, for example, at least 0.01 mm, at least 0.1 mm, or at least 1 mm, for example from about 0.01 to 5 mm, about 0.01 to 2 mm, about 0.01 to 1 mm, about 0.01 to 0.5 mm, about 0.02 to 0.45 mm, from about 0.05 to 0.3 mm, from about 0.075 to 0.25 mm, from about 0.1 to 0.2 mm, or from about 0.11 to 0.15 mm. The pore size of such sheets or strips may similarly vary within wide limits, for example from about 0.025 to 15 microns, or more specifically from about 0.1 to 3 microns; however, pore size is not intended to be a limiting factor in selection of the solid support. The flow rate of a solid support, where applicable, can also vary within wide limits, for example from about 12.5 to 90 sec/cm (i.e., 50 to 300 sec/4 cm), about 22.5 to 62.5 sec/cm (i.e., 90 to 250 sec/4 cm), about 25 to 62.5 sec/cm (i.e., 100 to 250 sec/4 cm), about 37.5 to 62.5 sec/cm (i.e., 150 to 250 sec/4 cm), or about 50 to 62.5 sec/cm (i.e., 200 to 250 sec/4 cm). In specific embodiments of devices described herein, the flow rate is about 62.5 sec/cm (i.e., 250 sec/4 cm). In other specific embodiments of devices described herein, the flow rate is about 37.5 sec/cm (i.e., 150 sec/4 cm).

In one example, the solid support is composed of an organic polymer. Suitable materials for the solid support include, but are not limited to: polypropylene, polyethylene, polybutylene, polyisobutylene, polybutadiene, polyisoprene, polyvinylpyrrolidine, polytetrafluoroethylene, polyvinylidene difluroide, polyfluoroethylene-propylene, polyethylenevinyl alcohol, polymethylpentene, polycholorotrifluoroethylene, polysulfornes, hydroxylated biaxially oriented polypropylene, aminated biaxially oriented polypropylene, thiolated biaxially oriented polypropylene, etyleneacrylic acid, thylene methacrylic acid, and blends of copolymers thereof).

In yet other examples, the solid support is a material containing, such as a coating containing, any one or more of or a mixture of the ingredients provided herein.

A wide variety of solid supports can be employed in accordance with the present disclosure. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

The solid support can be any format to which the molecule specific for the test agent can be affixed, such as microtiter plates, ELISA plates, test tubes, inorganic sheets, dipsticks, lateral flow devices, and the like. One example includes a linear array of molecules specific for the target agent, generally referred to in the art as a dipstick. Another suitable format includes a two-dimensional pattern of discrete cells (such as 4096 squares in a 64 by 64 array). As is appreciated by those skilled in the art, other array formats including, but not limited to slot (rectangular) and circular arrays are equally suitable for use. In one example, the array is formed on a polymer medium, which is a thread, membrane or film. An example of an organic polymer medium is a polypropylene sheet having a thickness on the order of about 1 mil. (0.001 inch) to about 20 mil., although the thickness of the film is not critical and can be varied over a fairly broad range.

In one example the format is a bead, such as a silica bead. In another example the format is a nitrocellulose membrane. In another example the format is filter paper. In yet another example the format is a glass slide. In one example, the solid support is a polypropylene thread. One or more polypropylene threads can be affixed to a plastic dipstick-type device; polypropylene membranes can be affixed to glass slides.

In one example the solid support is a microtiter plate. For example sensors can be affixed to the wells of a microtiter plate (for example wherein some wells can contain a sensor to detect target X, while other wells can contain a sensor to detect target Y; or several wells might include the same sensor, wherein multiple samples can be analyzed simultaneously). The test sample potentially containing a target of interest can be placed in the wells of a microtiter plate containing a sensor disclosed herein, and the presence of the target detected using the methods provided herein in. The microtiter plate format permits testing multiple samples simultaneously (together with controls) each in one or more different wells of the same plate; thus, permitting high-throughput analysis of numerous samples.

In some examples, a disclosed sensor is attached to more than one solid support. For example, a sensor containing an antibody specific for the target can be attached to a bead, which can then be attached to a conjugation pad of a lateral flow device.

Each of the supports and devices discussed herein (e.g., ELISA, lateral flow device) can be, in some embodiments, formatted to detect multiple targets by the addition of reagents specific for the other targets of interest. For example, certain wells of a microtiter plate can include molecules specific for the other targets of interest. Some lateral flow device embodiments can include secondary, tertiary or more capture areas containing molecules specific for the other targets of interest.

### Conjugating Materials to a Solid Support

Methods of conjugating a desired reagent (such as an antibody, glucose-1-phosphate, starting products, or nucleic acid aptamer) to the solid support (such as a reagent or conjugation pad) are conventional. The conjugation method used can be any chemistry that can covalently or non-covalently incorporate a desired reagent with other molecules. In some examples, a desired reagent (such as an antibody, glucose-1-phosphate, or nucleic acid) is attached to a solid support, such as a conjugation pad of a lateral flow device, simply by suspending the reagent in a solution, applying the solution to the pad, and allowing the solution to dry.

In one example the method uses a reaction that forms covalent bonds including but not limited to those between amines and isothiocyanates, between amines and esters, between amines and carboxyls, between thiols and maleimides, between thiols and thiols, between azides and alkynes, and between azides and nitriles. In another example, the method uses a reaction that forms non covalent interactions including but not limited to those between antibodies and antigens, and between organic chelators and metal ions.

### Lateral Flow Devices

In one example, the solid support is a lateral flow device, which can be used to determine the presence and/or amount of one or more target agents in a sample, such as a liquid sample. A lateral flow device is an analytical device having a test strip, through which flows a test sample fluid that is suspected of (or known to) containing a target agent. Lateral flow devices are useful to simplify and automate user sample interface and processing. One example of a lateral flow device is a glucose testing strip. Based on the principles of a glucose testing or pregnancy strip, lateral flow devices that incorporate the disclosed sensors can be developed. In some examples, by using such as lateral flow devices, samples can be directly contacted with or applied to the lateral flow device, and no further liquid transfer or mixing is required. Such devices can be used to detect target agents, for example qualitatively or quantitatively.

Lateral flow devices are commonly known in the art, and have a wide variety of physical formats. Any physical format that supports and/or houses the basic components of a lateral flow device in the proper function relationship is contemplated by this disclosure. In one example, the lateral flow devices disclosed in U.S. Pat. No. 7,799,554, Liu et al. (Angew. Chem. Int. Ed. 45:7955-59, 2006), Apilux et al. (Anal. Chem. 82:1727-32, 2010), Dungchai et al. (Anal. Chem. 81:5821-6, 2009), or Dungchai et al. (Analytica Chemica Acta 674:227-33, 2010) (all herein incorporated by reference) are used, such as one made using the Millipore Hi-Flow Plus Assembly Kit. There are a number of commercially available lateral flow type tests and patents disclosing methods for the detection of large analytes (MW greater than 1,000 Daltons) (see for example U.S. Pat. Nos. 5,229,073; 5,591,645; 4,168,146; 4,366,241; 4,855,240; 4,861,711; and 5,120,643; European Patent No. 0296724; WO 97/06439; and WO 98/36278). There are also lateral flow type tests for the detection of small-analytes (MW 100-1,000 Daltons) (see for example U.S. Pat. Nos. 4,703,017; 5,451,504; 5,451,507; 5,798,273; and 6,001,658). There are also lateral flow type tests for the detection of glucose (see for example U.S. Pat. Nos. 6,413,410 and 6,733,655).

The construction and design of lateral flow devices is very well known in the art, as described, for example, in Millipore Corporation, A Short Guide Developing Immunochromatographic Test Strips, 2nd Edition, pp. 1-40, 1999, available by request at (800) 645-5476; and Schleicher & Schuell, Easy to Work with BioScience, Products and Protocols 2003, pp. 73-98, 2003, 2003, available by request at Schleicher & Schuell BioScience, Inc., 10 Optical Avenue, Keene, N.H. 03431, (603) 352-3810; both of which are incorporated herein by reference.

Devices described herein generally include a strip of absorbent material (such as a microporous membrane), which can be made of different substances each joined to the other in zones, which may be abutted and/or overlapped. In some examples, the absorbent strip can be fixed on a supporting non-interactive material (such as nonwoven polyester), for example, to provide increased rigidity to the strip. Zones within each strip may differentially contain the reagents (such as starting products or antibodies) required for the detection and/or quantification of the particular target being tested for. Thus these zones can be viewed as functional sectors or functional regions within the test device.

These devices typically include a sample application area and one or more separate reagent areas in which desired reagents are immobilized (such as a sensor disclosed herein). For example, a lateral flow device containing at least two separate reagent areas (such as 2, 3, 4, 5, 6, 7, 8, 9, 10, 11, 12, 13, 14, 15 or more) can be used to detect a plurality of different target agents in a single sample. Any liquid (such as a fluid biological sample) applied in the sample application area flows along a path of flow from the sample application area, through the reagent areas, to the absorption area. Ultimately, ALP reacts with glucose-1-phosphate to form glucose, which flows to a downstream absorbent pad, which can act as a liquid reservoir. The resulting glucose on the lateral flow strip can be detected with a PGM, for example by insertion of the device into a PGM.

In one example where a lateral flow device can detect multiple targets, the device includes a single wicking pad or sample application area, and multiple conjugation or reagent pads, membranes or reaction pads, and absorption pads (such that one or more conjugation pads are associated with one or more particular membranes and an absorption pad). For example, each conjugation pad(s) can include a different reagents needed to detect a particular target agent. Thus, the glucose produced as a result of the target agent and present on each absorption pad can be used to detect the presence of a particular target agent.

The lateral flow device can include a wicking pad, conjugation pad, membrane, absorption pad, and combinations thereof. Such pads can abut one another or overlap, and can be attached to a backing. Exemplary materials that can be used for the components of a lateral flow device are shown in Table 1. However, one of skill in the art will recognize that the particular materials used in a particular lateral flow device will depend on a number of variables, including, for example, the target to be detected, the sample volume, the desired flow rate and others, and can routinely select the useful materials accordingly.

TABLE 1
Exemplary materials for a lateral flow device

| Component | Exemplary Material |
|---|---|
| Wicking/Sample Pad | Glass fiber |
| | Woven fibers |
| | Screen |
| | Non-woven fibers |
| | Cellulosic filters |
| | Paper |
| Conjugation/reagent Pad | Glass fiber |
| | Polyester |
| | Paper |
| | Surface modified polypropylene |
| Membrane/reaction pad | Nitrocellulose (including pure nitrocellulose |
| | and modified nitrocellulose) |
| | Nitrocellulose direct cast on polyester support |
| | Polyvinylidene fluoride |
| | Nylon |
| Absorption Pad | Cellulosic filters |
| | Paper |

The test sample is applied to or contacted with the wicking pad (which is usually at the proximal end of the device, but can for example be at the center of the device for example when multiple conjugation pads are included to detect multiple targets), for instance by dipping or spotting. A sample is collected or obtained using methods well known to those skilled in the art. The sample containing the test agent to be detected may be obtained from any source. In some examples, the sample is diluted, purified, concentrated, filtered, dissolved, suspended or otherwise manipulated prior to assay. The fluid sample migrates distally through all the functional regions of the strip. The final distribution of the fluid in the individual functional regions depends on the adsorptive capacity and the dimensions of the materials used.

The wicking pad ensures that the sample moves through the device in a controllable manner, such that it flows in a unilateral direction. The wicking pad initially receives the sample, and can serve to remove particulates from the sample. Among the various materials that can be used to construct a sample pad (see Table 1), a cellulose sample pad may be beneficial if a large bed volume (e.g., 250 μl/cm²) is a factor in a particular application. In one example, the wicking pad is made of Millipore cellulose fiber sample pads (such as a 10 to 25 mm pad, such as a 15 mm pad). Wicking pads may be treated with one or more release agents, such as buffers, salts, proteins, detergents, and surfactants. Such release agents may be useful, for example, to promote resolubilization of conjugate-pad constituents, and to block non-specific binding sites in other components of a lateral flow device, such as a nitrocellulose membrane. Representative release agents include, for example, trehalose or glucose (1%-5%), PVP or PVA (0.5%-2%), Tween 20 or Triton X-100 (0.1%-1%), casein (1%-2%), SDS (0.02%-5%), and PEG (0.02%-5%). In one example the wicking pad includes reagents to lyse RBCs, such as one containing ammonium chloride, potassium bicarbonate and EDTA, such ammonium chloride (8.26 g), potassium bicarbonate (1 g) and EDTA (0.037 g) in 1 liter water. (0.037 g) in 1 liter water.

After contacting the sample to the wicking pad, the sample liquid migrates from bottom to the top because of capillary force (or from the center outwards). The sample then flows to a first conjugation or reagent pad, which serves to, among other things, hold at least some of the reagents needed to convert the target into a corresponding glucose signal. The reagents can be immobilized to the conjugation pad by spotting (for example the reagents, such as an antibody, aptamer, ALP, glucose-1-phosphate, or starting materials, can be suspended in water or other suitable buffer and spotted onto the conjugation pad and allowed to dry). The conjugation pad can be made of known materials (see Table 1), such as glass fiber, such as one that is 10 to 25 mm, for example 13 mm. When the sample reaches the conjugation pad, target agent present in the sample can interact with the reagents immobilized to the conjugation pad. In some examples, reactions, such as binding reactions, occur on reaction pads. In some examples, the sample passes through a series of reagent and reaction/membrane pads. The reaction pad can be made of known materials (see Table 1), such as a HiFlow Plus Cellulose Ester Membrane, such as one that is 10 to 40 mm, for example 25 mm. Finally, the glucose produced moves with the flow and reaches the absorption pad, where it is then detected by a connected PGM (for example it can be attached to a commercially available glucose strip, such as one containing glucose oxidase). The absorbent pad acts to draw the sample across the conjugation pad(s) and membrane(s) by capillary action and collect it. This action is useful to insure the sample solution will flow from the sample or wicking pad unidirectionally through conjugation pad and the membrane to the absorption pad. Any of a variety of materials is useful to prepare an absorbent pad, see, for example, Table 1. In some device embodiments, an absorbent pad can be paper (i.e., cellulosic fibers). One of skill in the art may select a paper absorbent pad on the basis of, for example, its thickness, compressibility, manufacturability, and uniformity of bed volume. The volume uptake of an absorbent made may be adjusted by changing the dimensions (usually the length) of an absorbent pad. In one example the absorption is one that is 10 to 25 mm, for example 15 mm.

The amount of glucose detected by the PGM, and target agent are proportional o inversely proportional to each other, thus the target agent can be quantified by the read out of glucose meter. The original glucose concentration in the sample can be subtracted from the result for more accurate quantification of the target agent. Because of high selectivity of the reagents used for its target, interference by other components in the sample is minimal.

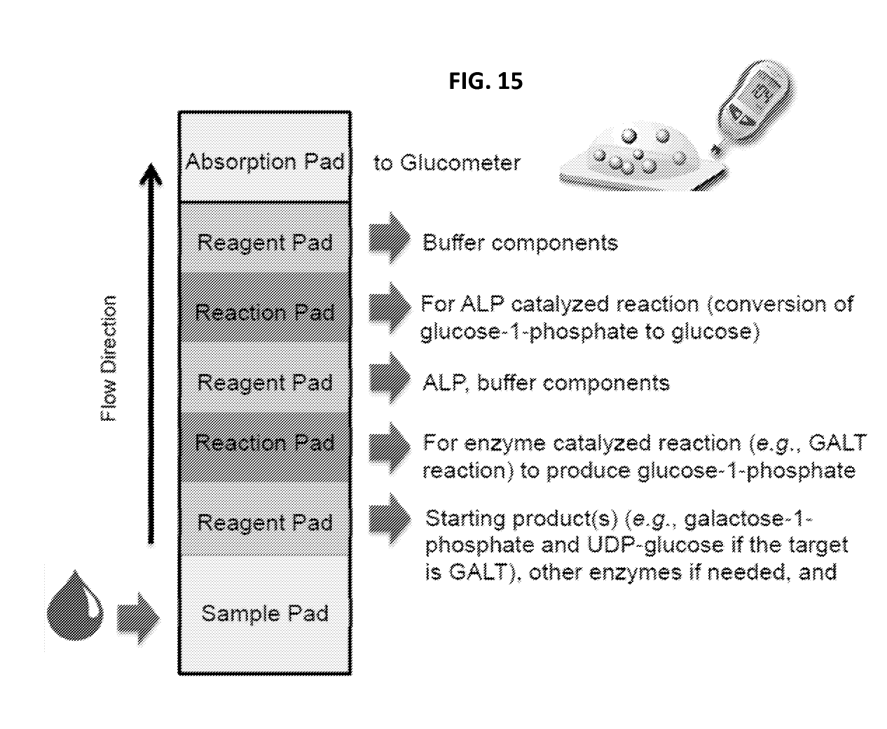

A specific exemplary lateral flow device is shown in FIG. 15 for detecting a broad range of non-glucose targets. The lateral flow device can include a bibulous lateral flow strip, which can be present in housing material (such as plastic or other material). In one example, the lateral flow device is similar to a glucose test strip. FIG. 15 shows a lateral flow device that can be read by a PGM.

The lateral flow strip is divided into a sections, which can include a proximal wicking pad, multiple conjugation or reagent pads (containing immobilized reagents), optional multiple membrane or reaction pads (can be used to allow time for the reagents and the sample to react and interact), and a distal absorption pad. The flow path along strip passes from proximal wicking pad, through the reagent and reaction pads, for eventual collection in an absorption pad.

As shown in FIG. 15, the sample containing or suspected of containing one or more target agents (such as target enzymes) is applied to the sample pad. If desired, liquid can be added to the sample, or the sample can be concentrated, before applying it to the sample pad. In one example, a fluid sample containing or suspected of containing a target of interest, is applied to the sample/wicking pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. In some examples wherein the sample is whole blood, the sample/wicking pad can include one or more reagents that result in lysis of the red blood cells, such as a sample/wicking pad containing is ammonium chloride, potassium bicarbonate and EDTA. The sample pad ensures a controllable (unilateral) flow of the sample. The sample migrates from the bottom to the top of the lateral flow device following the indicated flow direction in FIG. 15 because of capillary force.

The first reaction pad includes the one or more starting products, wherein at least one of the starting products can be converted to glucose-1-phosphate by a target enzyme, or includes one or more enzymes involved in the production of glucose-1-phosphate, or combinations thereof. These one or more starting products are immobilized to the first reaction pad. In an example where the target enzyme includes galactose-1-phosphate uridylyltransferase (GALT), the one or more starting products include UDP-glucose and galactose-1-phosphate. In an example where the target enzyme includes glycogen phosphorylase (GP), the one or more starting products include glycogen and 5′-adenosine monophosphate (AMP). In an example where the target enzyme includes galactokinase, the one or more starting products include α-D-galactose, UDP-glucose, and GALT. In an example where the target enzyme includes UDP-glucose 4-epimerase, the one or more starting products include UDP-galactose, galactose-1-phosphate and GALT.

From the wicking pad, the sample passes, for instance by capillary action, to the first reagent pad. When the sample reaches the first reagent pad, it transports the one or more starting products to the first reaction pad. In the first reaction pad, the one or more enzymatic reactions that eventually produce glucose-1-phosphate occur. For example, if the target is GP and it is present in the sample, it will react with the glycogen and 5′-adenosine monophosphate (AMP) to generate glucose-1-phosphate. After this reaction, the resulting glucose-1-phosphate subsequently flows to the second reagent pad. The second reagent pad contains immobilized ALP and optionally buffer components. The ALP and glucose-1-phosphate move with the flow to the second reaction pad, where the ALP converts the glucose-1-phosphate to glucose. The resulting glucose moves with the flow to the third reagent pad. The third reagent pad includes one or more agents that can neutralize a pH from basic to neutral (such as an acidic buffer or reagents). The resulting glucose can subsequently flow to the absorption pad, which can be detected by a glucose meter, such as a PGM. The presence of glucose indicates the presence of target agent in the sample tested. In some examples, the amount of glucose detected by the PGM is proportional to the amount of test enzyme or test enzyme activity in the test sample. This permits quantification of the test enzyme or test enzyme activity by the read-out of glucose meter. The original glucose concentration in the sample can be subtracted from the result if desired.

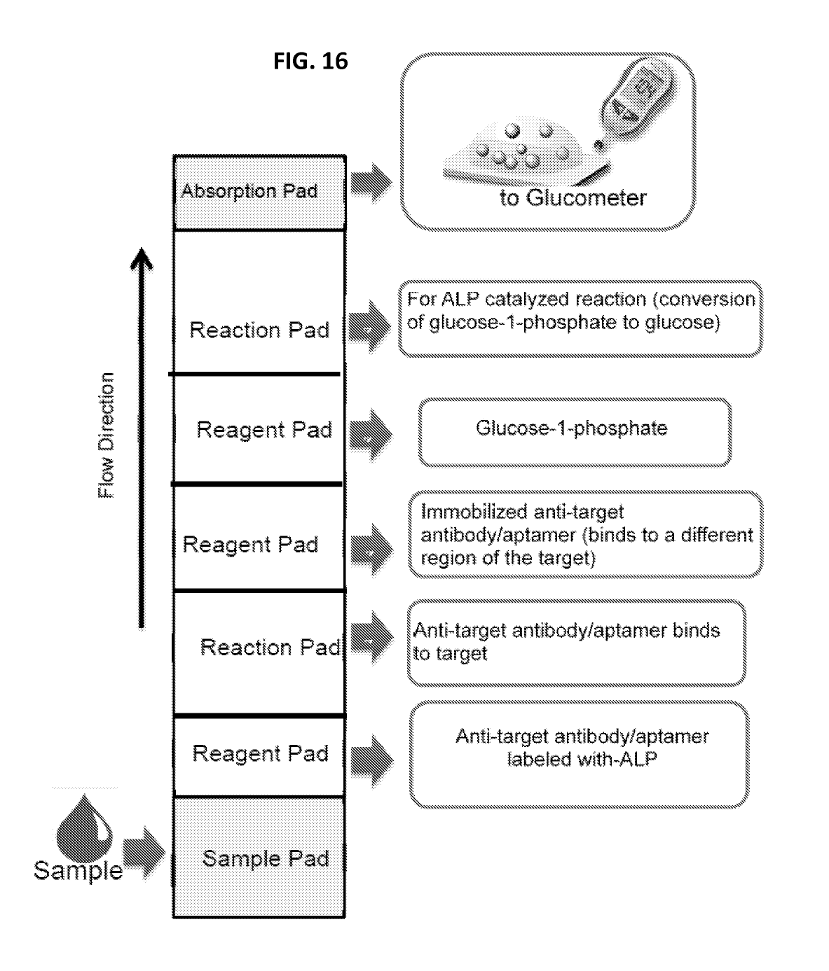

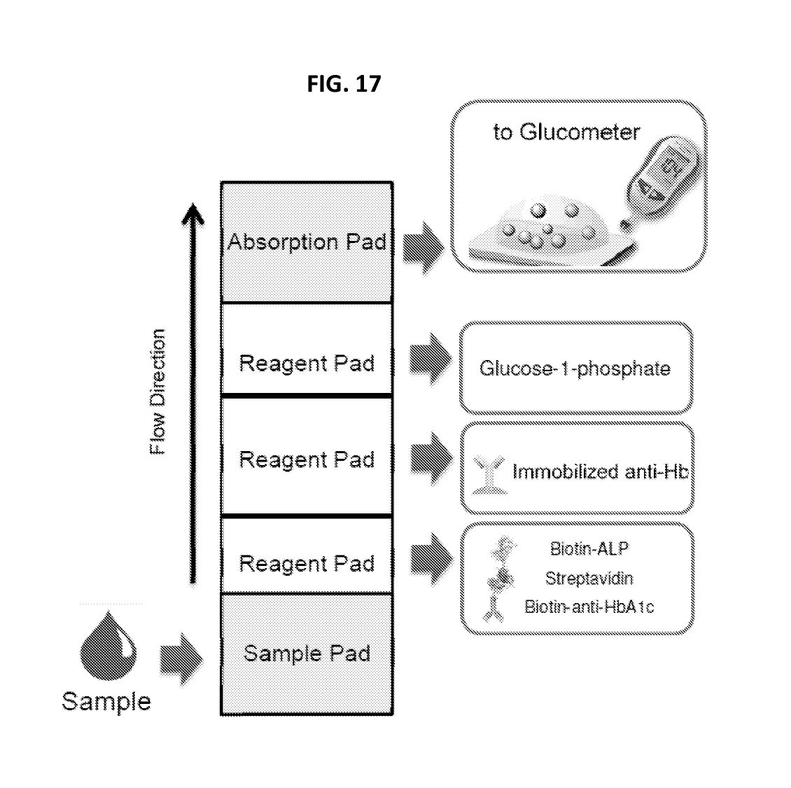

Other exemplary lateral flow devices are shown in FIGS. 16 and 17, for example that can be used when the target agents can bind a specific antibody or aptamer. FIG. 16 shows a lateral flow device that can be read by a PGM for detecting a broad range of non-glucose targets in many different samples, using a lateral flow device containing one or more ALP-conjugated antibodies. ALP can convert glucose-1-phosphate into glucose. As shown in FIG. 16, the lateral flow device contains wicking or sample pad, multiple reagent/conjugation pads (which can be interspersed with membranes/reaction pads where the desired interactions are given time to occur) and an absorption pad. The sample containing or suspected of containing one or more target agents is applied to the sample pad. If desired, liquid can be added to the sample, or the sample can be concentrated, before applying it to the sample pad. The sample pad ensures a controllable (unilateral) flow of the sample. The sample migrates from the bottom to the top of the lateral flow device following the indicated flow direction in FIG. 16 because of capillary force. When the sample reaches the reagent pad, which contains ALP-labeled first antibodies or aptamers specific for the target, target agent present in the sample binds to the ALP-antibodies/aptamers, for example on a reaction pad. The resulting ALP-first antibody-target complex moves with the flow to the second reagent pad containing immobilized second antibodies or aptamers specific for a different region of the target (such as a different epitope). The ALP-first antibody-target complex is allowed to interact with the second antibody/aptamer, for example on a reaction pad. Thus, if present, the target and the first ALP-antibody/aptamer are captured to the solid phase (for example on the second reagent pad) rather than remaining in the mobile phase (flow solution). Materials in the mobile phase move to the area containing glucose-1-phosphate. If ALP is present in the mobile phase, it will convert the glucose-1-phosphate to glucose, which flows to the absorption pad where it is then detected by a PGM. The more target agent present in the sample, the more ALP will be captured to the solid phase rather than remaining in the mobile phase (flow solution) and the less glucose signal will be detected. Thus, the amount of glucose detected by the PGM is inversely proportional to the amount of target in the test sample. This permits quantification of the target by the read-out of glucose meter. The original glucose concentration in the sample can be subtracted from the result if desired.

Another specific lateral flow device, for example one that can be used in a competitive assay, is shown in FIG. 20. Although FIG. 20 shows ALP-target conjugate and Ab-MBs, these can be substituted with ALP-antibody conjugate and target MBs, respectively, as shown in FIG. 19B. In addition, one skilled in the art will appreciate that besides antibodies, other target-specific binding agents can be used, such as aptamers, and that other solid supports can be used instead of MBs. As shown in FIG. 20, the sample containing or suspected of containing one or more target agents is applied to the sample pad. If desired, liquid can be added to the sample, or the sample can be concentrated, before applying it to the sample pad. In one example, a fluid sample containing or suspected of containing a target of interest, is applied to the sample/wicking pad, for example dropwise or by dipping the end of the device into the sample. If the sample is whole blood, an optional developer fluid can be added to the blood sample to cause hemolysis of the red blood cells and, in some cases, to make an appropriate dilution of the whole blood sample. In some examples wherein the sample is whole blood, the sample/wicking pad can include one or more reagents that result in lysis of the red blood cells, such as a sample/wicking pad containing is ammonium chloride, potassium bicarbonate and EDTA. The sample pad ensures a controllable (unilateral) flow of the sample. The sample migrates from the bottom to the top of the lateral flow device following the indicated flow direction in FIG. 20 because of capillary force.

The first reagent pad includes ALP-target conjugates (or alternatively ALP-antibody or aptamer conjugates). From the sample pad, the sample passes, for instance by capillary action, to the first reagent pad. When the sample reaches the reagent pad, the solution transports the ALP-target conjugates (or alternatively ALP-antibody or aptamer conjugates) to a second reaction pad. The second reaction pad contains immobilized Ab—or aptamer—MBs (or target MBs). At the second reaction pad, target in the sample, ALP-target conjugates (or alternatively ALP-antibody or aptamer conjugates) and Ab-MBs or (target MBs) initiate the competitive binding. In the presence of target, targets will bind Ab-MBs (as shown in the dash-lined rectangle), thus allowing ALP conjugates to reach a subsequent interface pad containing glucose-1-phosphate to produce glucose for glucose meter measurement (or targets will bind ALP-antibody or aptamer conjugates which can move to the interface pad and result in glucose production). One skilled in the art will appreciate that the opposite result can be achieved if instead of the solid supports (e.g., MBs) being immobilized such that they cannot reach the glucose-1-phosphate containing pad, the ALP conjugates are immobilized or otherwise prevented from reaching the glucose-1-phosphate containing pad, in which case the amount of glucose detected will be inversely proportional to the amount of target in the sample.

### Microfluidic Devices

In one example, the solid support is a microfluidic device, which can be used to determine the presence and/or amount of one or more target agents in a sample, such as a liquid sample. Such devices are also referred to as “lab-on-a-chip” devices. The development of microfluidics and microfluidic techniques has provided improved chemical and biological research tools, including platforms for performing chemical reactions, combining and separating fluids, diluting samples, and generating gradients (for example, see U.S. Pat. No. 6,645,432).

A portable microfluidic device can be transported to almost any location. For microfluidic assays and devices, test samples (such as a liquid sample) can be supplied by an operator, for example using a micropipette. A test sample can be introduced into an inlet of a microfluidic system and the fluid may be drawn through the system by application of a vacuum source to the outlet end of the microfluidic system. Reagents may also be pumped in, for instance by using different syringe pumps filled with the required reagents. After one fluid is pumped into the microfluidic device, a second can be pumped in by disconnecting a line from the first pump and connecting a line from a second pump. Alternatively, valving may be used to switch from one pumped fluid to another. Different pumps can be used for each fluid to avoid cross contamination, for example when two fluids contain components that may react with each other or, when mixed, can affect the results of an assay or reaction. Continuous flow systems can use a series of two different fluids passing serially through a reaction channel. Fluids can be pumped into a channel in serial fashion by switching, through valving, the fluid source that is feeding the tube. The fluids constantly move through the system in sequence and are allowed to react in the channel.

Microfluidic devices for analyzing a target analyze are well known, and can be adapted using the disclosed ALP system to detect a target of interest. For example devices from Axis Shield (Scotland), such as the Afinion analyzer, analyzers from Claros (Woburn, Mass.), and devices from Advanced Liquid Logic (Morrisville, N.C.) such as those based on electrowetting. Other exemplary devices are described in US Patent Publication Nos. 20110315229; 20100279310; 2012001830 and 2009031171.

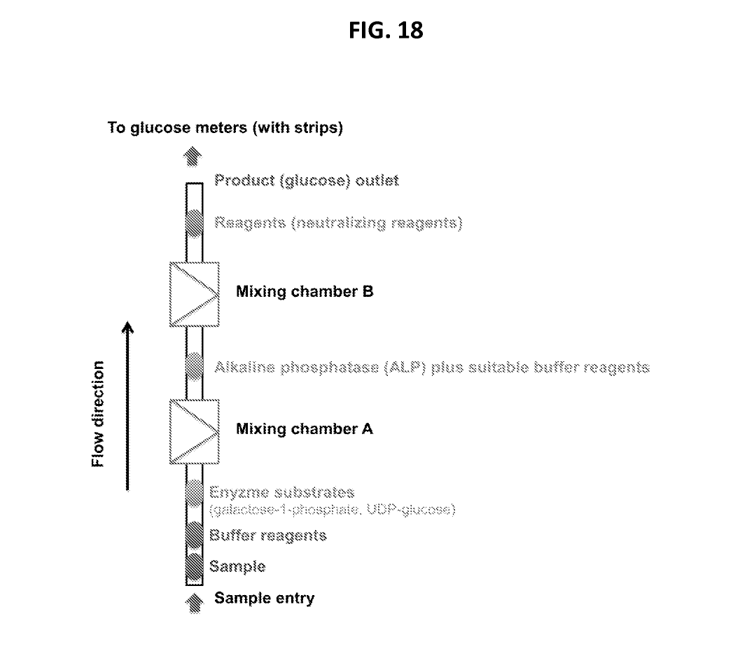

An exemplary microfluidic device that can be used with the methods provided herein is shown in FIG. 18. The microfluidic device controls the movement of the sample and other liquids, dispenses reagents, and merges or splits a micro-size droplet in the microfluidic device via the voltage applied to the flow versus the device. As shown in the FIG. 18, the device includes a sample entry port, where the sample is introduced into the device. The device also includes an area containing buffer reagents and an area containing one or more enzyme substrates or starting products (wherein at least one of the starting products can be converted to glucose-1-phosphate by a target enzyme, comprises an enzyme involved in the production of glucose-1-phosphate, or combinations thereof). The device includes one more mixing chambers, where desired reactions can occur. In one example, the device includes a first mixing chamber (chamber A in FIG. 18) where glucose-1-phosphate is produced from the interaction between the starting products and the target enzyme. The device also includes a region upstream of the first mixing chamber, containing ALP and buffers. The device includes a region containing ALP, for example between the first mixing chamber and a second mixing chamber. The product from the first mixing chamber (e.g., glucose-1-phosphate) passes thru the region containing ALP, and enters the second reaction chamber (chamber B) where glucose is produced from the interaction between the glucose-1-phosphate and ALP. The device can further include a region containing reagents that neutralize the pH of the droplet released from the second mixing chamber, such as NaH₂PO₄). Although a particular configuration is shown in FIG. 18, one skilled in the art will appreciate that other configuration as possible, for example more regions or mixing chambers if multiple targets are to be detected in the same sample on the same device. For example the device can have discrete regions and mixing chambers for each target to be detected. In such an example, the microfluidic device may include multiple exit ports, one for each target.

The device can be used as follows. The sample (such as a blood or urine sample) is introduced into the microfluidic device and mixed with droplets of buffer reagents (such as red blood cell lysis buffers and suitable buffers for the enzymatic reaction) and starting products or enzyme substrates. In an example where the target enzyme includes galactose-1-phosphate uridylyltransferase (GALT), the one or more starting products include UDP-glucose and galactose-1-phosphate. This is shown in FIG. 18, but one skilled in the art will recognize that other substrates can be included depending on the target enzyme. In an example where the target enzyme includes glycogen phosphorylase (GP), the one or more starting products include glycogen and 5′-adenosine monophosphate (AMP). In an example where the target enzyme includes galactokinase, the one or more starting products include α-D-galactose, UDP-glucose, and GALT. In an example where the target enzyme includes UDP-glucose 4-epimerase, the one or more starting products include UDP-galactose, galactose-1-phosphate and GALT.

The mixture droplet moves into a first mixing chamber (chamber A) for sufficient time to ensure completion of the enzymatic reaction (e.g., production of glucose-1-phosphate from the starting products by the target enzyme). After the reaction, the droplet merges with ALP and enters a second reaction chamber (chamber B) to convert glucose-1-phosphate into glucose. Finally, the droplet merges with the reagents (e.g., neutralization reagents such as NaH₂PO₄) located after the mixing chamber B, and the droplet is tested by a glucose meter (e.g., PGM) after it is released from the microfluidic device.

### Target Agents

The disclosed sensors and methods can be designed to detect any target agent of interest. Thus, the methods and devices provided herein can be used to detect any target agent of interest, such as the specific examples provided herein. Exemplary target agents are provided below; however one skilled in the art will appreciate that other target agents can be detected.

Target Agents that Produce Glucose-1-Phosphate

In one example, the target is an agent, such as an enzyme, that can convert one or more starting products to glucose-1-phosphate. As described above, selecting an appropriate recognition molecule that permits detection of the target agent, allows one to develop a sensor that can be used to detect a particular target agent.

Exemplary targets include, but are not limited to: galactose-1-phosphate uridylyltransferase (GALT), glycogen phosphorylase (GP); galactokinase; and UDP-glucose 4-epimerase.

Target Agents that Specifically Bind an Antibody or Aptamer

In one example, the target is any agent that can specifically bind to a particular antibody or aptamer. Commercially available antibodies are available for numerous agents, such as proteins (e.g., cytokines, tumor antigens, etc.), metals, and nucleic acid molecules. In addition, methods of making antibodies and aptamers are well known in the art.

Aptamers are nucleic acids (such as DNA or RNA) that recognize targets with high affinity and specificity. In vitro selection methods can be used to obtain aptamers for a wide range of target molecules with exceptionally high affinity, having dissociation constants as high as in the picomolar range (Brody and Gold, J. Biotechnol. 74: 5-13, 2000; Jayasena, Clin. Chem., 45:1628-1650, 1999; Wilson and Szostak, Annu. Rev. Biochem. 68: 611-647, 1999). For example, aptamers have been developed to recognize metal ions such as Zn(II) (Ciesiolka et al., RNA 1: 538-550, 1995) and Ni(II) (Hofmann et al., RNA, 3:1289-1300, 1997); nucleotides such as adenosine triphosphate (ATP) (Huizenga and Szostak, Biochemistry, 34:656-665, 1995); and guanine (Kiga et al., Nucleic Acids Research, 26:1755-60, 1998); co-factors such as NAD (Kiga et al., Nucleic Acids Research, 26:1755-60, 1998) and flavin (Lauhon and Szostak, J. Am. Chem. Soc., 117:1246-57, 1995); antibiotics such as viomycin (Wallis et al., Chem. Biol. 4: 357-366, 1997) and streptomycin (Wallace and Schroeder, RNA 4:112-123, 1998); proteins such as HIV reverse transcriptase (Chaloin et al., Nucleic Acids Research, 30:4001-8, 2002) and hepatitis C virus RNA-dependent RNA polymerase (Biroccio et al., J. Virol. 76:3688-96, 2002); toxins such as cholera whole toxin and staphylococcal enterotoxin B (Bruno and Kiel, BioTechniques, 32: pp. 178-180 and 182-183, 2002); and bacterial spores such as the anthrax (Bruno and Kiel, Biosensors & Bioelectronics, 14:457-464, 1999). Compared to antibodies, DNA/RNA based aptamers are easier to obtain and less expensive to produce because they are obtained in vitro in short time periods (days vs. months) and with limited cost. In addition, DNA/RNA aptamers can be denatured and renatured many times without losing their biorecognition ability.

Metals

Antibodies specific for particular metals are known in the art. For example, Zhu et al. describe mAbs specific for chelated cadmium ions (J. Agric. Food Chem. 55:7648-53, 2007), Wylie et al. describe mAbs specific for mercuric ions (PNAS 89:4104-8, 1992), and Love et al. describe mAbs specific for inidium (Biochem. 32:10950-9, 1993). In addition, bifunctional derivatives of metal ion chelators (EDTA, DTPA, DOTA) can be covalently conjugated to proteins and loaded with the desired metal ion. These conjugates can be used to prepare hybridoma cell lines which synthesize metal-specific monoclonal antibodies.

In one example the target agent is a metal (e.g., elements, compounds, or alloys that have high electrical conductivity), such as a heavy metal or a nutritional metal. Exemplary metals that can be detected using the methods provided herein include but are not limited to: Pb²⁺, Zn²⁺, Cd²⁺, Hg²⁺, UO₂²⁺, Mg²⁺, Cu²⁺. Metals occupy the bulk of the periodic table, while non-metallic elements can only be found on the right-hand-side of the Periodic Table of the Elements. A diagonal line drawn from boron (B) to polonium (Po) separates the metals from the nonmetals. Most elements on this line are metalloids, sometimes called semiconductors. Elements to the lower left of this division line are called metals, while elements to the upper right of the division line are called non-metals.

Heavy metals include any metallic chemical element that has a relatively high density and is toxic, highly toxic or poisonous at low concentrations. Examples of heavy metals include mercury (Hg), cadmium (Cd), arsenic (As), chromium (Cr), thallium (Tl), uranium (U), plutonium (Pu), and lead (Pb).

Nutritional metal ions include those important in animal nutrition and may be necessary for particular biological functions, include calcium, iron, cobalt, magnesium, manganese, molybdenum, zinc, cadmium, and copper.

Pathogens/Microbes

Antibodies specific for particular microbes are known in the art. Usually, such antibodies recognize a surface protein expressed by the microbe. For example, such antibodies are available from commercial sources, such as Novus Biologicals (Littleton, Colo.) and ProSci Incorporated (Poway, Calif.) provide E. coli-specific antibodies; KPL (Gaithersburg, Md.) provides Listeria-specific antibodies; Thermo Scientific/Pierce Antibodies (Rockford, Ill.) provides antibodies specific for several microbes, including bacteria and viruses, such as influenza A, HIV, HSV 1 and 2, E. coli, Staphylococcus aureus, Bacillus anthracis and spores thereof, Plasmodium, and Cryptosporidium.

Any pathogen or microbe can be detected using the sensors and methods provided herein. For example, particular antimicrobial antigens, as well as bacterial spores, can be detected. In some examples, a particular microbial cell is detected, or a particular virus. In some examples, intact microbes are detected, for example by detecting a target surface protein (such as a receptor) using sensors that include for example antibodies specific for the target protein.

Exemplary pathogens include, but are not limited to, viruses, bacteria, fungi, nematodes, and protozoa. A non-limiting list of pathogens that can be detected using the methods and sensors provided herein are provided below.

For example, viruses include positive-strand RNA viruses and negative-strand RNA viruses. Exemplary positive-strand RNA viruses include, but are not limited to: Picornaviruses (such as Aphthoviridae [for example foot-and-mouth-disease virus (FMDV)]), Cardioviridae; Enteroviridae (such as Coxsackie viruses, Echoviruses, Enteroviruses, and Polioviruses); Rhinoviridae (Rhinoviruses)); Hepataviridae (Hepatitis A viruses); Togaviruses (examples of which include rubella; alphaviruses (such as Western equine encephalitis virus, Eastern equine encephalitis virus, and Venezuelan equine encephalitis virus)); Flaviviruses (examples of which include Dengue virus, West Nile virus, and Japanese encephalitis virus); Calciviridae (which includes Norovirus and Sapovirus); and Coronaviruses (examples of which include SARS coronaviruses, such as the Urbani strain).

Exemplary negative-strand RNA viruses include, but are not limited to: Orthomyxyoviruses (such as the influenza virus), Rhabdoviruses (such as Rabies virus), and Paramyxoviruses (examples of which include measles virus, respiratory syncytial virus, and parainfluenza viruses).

Viruses also include DNA viruses. DNA viruses include, but are not limited to: Herpesviruses (such as Varicella-zoster virus, for example the Oka strain; cytomegalovirus; and Herpes simplex virus (HSV) types 1 and 2), Adenoviruses (such as Adenovirus type 1 and Adenovirus type 41), Poxviruses (such as Vaccinia virus), and Parvoviruses (such as Parvovirus B19).

Another group of viruses includes Retroviruses. Examples of retroviruses include, but are not limited to: human immunodeficiency virus type 1 (HIV-1), such as subtype C; HIV-2; equine infectious anemia virus; feline immunodeficiency virus (FIV); feline leukemia viruses (FeLV); simian immunodeficiency virus (SIV); and avian sarcoma virus.

In one example, the virus detected with the disclosed methods is one or more of the following: HIV (for example an HIV antibody, p24 antigen, or HIV genome); Hepatitis A virus (for example an Hepatitis A antibody, or Hepatitis A viral genome); Hepatitis B (HB) virus (for example an HB core antibody, HB surface antibody, HB surface antigen, or HB viral genome); Hepatitis C(HC) virus (for example an HC antibody, or HC viral genome); Hepatitis D (HD) virus (for example an HD antibody, or HD viral genome); Hepatitis E virus (for example a Hepatitis E antibody, or HE viral genome); a respiratory virus (such as influenza A & B, respiratory syncytial virus, human parainfluenza virus, or human metapneumovirus), or West Nile Virus.

Pathogens also include bacteria. Bacteria can be classified as gram-negative or gram-positive. Exemplary gram-negative bacteria include, but are not limited to: Escherichia coli (e.g., K-12 and O157:H7), Shigella dysenteriae, and Vibrio cholerae. Exemplary gram-positive bacteria include, but are not limited to: Bacillus anthracis, Staphylococcus aureus, Listeria, pneumococcus, gonococcus, and streptococcal meningitis. In one example, the bacteria detected with the disclosed methods is one or more of the following: Group A Streptococcus; Group B Streptococcus; Helicobacter pylori; Methicillin-resistant Staphylococcus aureus; Vancomycin-resistant enterococci; Clostridium difficile; E. coli (e.g., Shiga toxin producing strains); Listeria; Salmonella; Campylobacter; B. anthracis (such as spores); Chlamydia trachomatis; and Neisseria gonorrhoeae.

Protozoa, nemotodes, and fungi are also types of pathogens. Exemplary protozoa include, but are not limited to, Plasmodium (e.g., Plasmodium falciparum to diagnose malaria), Leishmania, Acanthamoeba, Giardia, Entamoeba, Cryptosporidium, Isospora, Balantidium, Trichomonas, Trypanosoma (e.g., Trypanosoma brucei), Naegleria, and Toxoplasma. Exemplary fungi include, but are not limited to, Coccidiodes immitis and Blastomyces dermatitidis.

In one example, bacterial spores are detected. For example, the genus of Bacillus and Clostridium bacteria produce spores that can be detected. Thus, C. botulinum, C. perfringens, B. cereus, and B. anthracis spores can be detected (for example detecting anthrax spores). One will also recognize that spores from green plants can also be detected using the methods and devices provided herein.

Proteins

The disclosed methods and sensors permit detection of a variety of proteins, such as cell surface receptors, cytokines, antibodies, hormones, as well as toxins. In some examples, a target protein is selected that is associated with a disease or condition, such that detection (or absence) of the target protein can be used to infer information (such as diagnostic or prognostic information for the subject from whom the sample is obtained) relating to the disease or condition. Antibodies specific for particular proteins are known in the art. For example, such antibodies are available from commercial sources, such as Invitrogen, Santa Cruz Biotechnology (Santa Cruz, Calif.); ABCam (Cambridge, Mass.) and IBL International (Hamburg, Germany).

In one example the target protein is a cytokine. Cytokines are small proteins secreted by immune cells that have effects on other cells. Examples include interleukins (IL) and interferons (IFN), and chemokines, such as IL-1, IL-2, IL-4, IL-6, IL-8, IL-10, IFN-γ, IFN-β, transforming growth factor (TGF-β), and tumor necrosis factor (TNF)-α.

In one example the target protein is a hormone. A hormone is a chemical messenger that transports a signal from one cell to another. Examples include plant and animal hormones, such as endocrine hormones or exocrine hormones. Particular examples include follicle stimulating hormone (FSH), human chorionic gonadotropin (hCG), thyroid stimulating hormone (TSH), growth hormone, progesterone, and the like.

In yet another example the target protein is a toxin. Toxins are poisonous substances produced by cells or organisms, such as plants, animals, microorganisms (including, but not limited to, bacteria, viruses, fungi, rickettsiae or protozoa). Particular examples include botulinum toxin, ricin, diphtheria toxin, Shiga toxin, Cholera toxin, Staphylococcal enterotoxin B, an endotoxin, and anthrax toxin. In another example, the toxin is an environmental toxin. In one example the toxin is a mycotoxin, such as: aflatoxin, citrinin, ergot alkaloids, patulin, fusarium toxins, or ochratoxin A.

In one example, the target protein is a tumor-associated or tumor-specific antigen, such as CA-125 (ovarian cancer marker), alphafetoprotein (AFP, liver cancer marker); carcinoembryonic antigen (CEA; bowel cancers), BRCA1 and 2 (breast cancer), and the like.

In one example the target protein is a fertility-related biomarker, such as hCG, luteinizing hormone (LH), follicle-stimulating hormone (FSH), or fetal fibrinogen.

In another example, the target protein is one found on the surface of a target microbe or cell, such as a bacterial cell, virus, spore, or tumor cell. Such proteins, such as receptors, may be specific for the microbe or cell (for example HER2, IGF1R, EGFR or other tumor-specific receptor).

In one example the target protein is a diagnostic protein, such as prostate-specific antigen (PSA, for example GenBank Accession No. NP_{—}001025218), C reactive protein, cyclic citrullinate peptides (CCP, for example to diagnose rheumatoid arthritis) or glycated hemoglobin (Hb A1c).

Recreational and Other Drugs

The disclosed methods and sensors permit detection of a variety of drugs, such as pharmaceutical or recreational drugs. Antibodies specific for particular proteins are known in the art. For example, antibodies to tetrahydrocannabinol, heroin, cocaine, caffeine, and methamphetamine are available from AbCam (Cambridge, Mass.).

For example, the presence of caffeine, cocaine, opiates and opioids (such as oxycodone), cannabis (for example by detecting tetrahydrocannabinol (THC)), heroin, methamphetamines, crack, ethanol, acetaminophen, benzodiazepines, methadone, phencyclidine, or tobacco (for example by detecting nicotine), can be detected using the disclosed methods, sensors and devices.

In one example, the target is a therapeutic drug, such as theophylline, methotrexate, tobramycin, cyclosporine, rapamycin, or chloramphenicol.

Cells

The disclosed methods and sensors permit detection of a variety of cells, such as tumor or cancer cells, as well as other diseased cells. In one example, the methods and sensors can distinguish between a tumor cell and a normal cell of the same cell type, such as a normal breast cell from a cancerous breast cell. Tumors are abnormal growths which can be malignant or benign, solid or liquid (for example, hematogenous). In some examples, cells are detected by using a sensor that includes an antibody specific for a surface protein, such as a receptor on the surface of the cell.

Antibodies specific for particular cells are known in the art. Usually, such antibodies recognize a surface protein expressed by the cell, such as a receptor. For example, such antibodies are available from commercial sources, such as AbCam and Santa Cruz Biotechnology.

In one example, the target cell is a hematological tumor cell, such as: leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (including low-, intermediate-, and high-grade), multiple myeloma, Waldenström's macroglobulinemia, heavy chain disease, myelodysplastic syndrome, mantle cell lymphoma and myelodysplasia.

In one example, the target cell is a solid tumor cell. Examples of solid tumors, such as sarcomas and carcinomas, include, but are not limited to: fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyogioma, ependymoma, pinealoma, hemangioblastoma, acoustic neuroma, oligodendroglioma, menangioma, melanoma, neuroblastoma and retinoblastoma).

### Kits

The disclosure also provides kits that include one or more of the sensors disclosed herein, for example sensors that are part of a lateral flow device. For example, a kit can include at least 2 different sensors permitting detection of at least two different target agents, such as at least 3, at least 4, at least 5, or at least 10 different sensors. In a specific example, a kit can include at least 2 different lateral flow devices permitting detection of at least two different target agents, such as at least 3, at least 4, at least 5, or at least 10 different lateral flow devices.

The kits can the sensor or lateral flow device and a carrier means, such as a box, a bag, a satchel, plastic carton (such as molded plastic or other clear packaging), wrapper (such as, a sealed or sealable plastic, paper, or metallic wrapper), or other container. In some examples, kit components will be enclosed in a single packaging unit, such as a box or other container, which packaging unit may have compartments into which one or more components of the kit can be placed. In other examples, a kit includes one or more containers, for instance vials, tubes, and the like that can retain, for example, one or more biological samples to be tested, positive and/or negative control samples or solutions (such as, a positive control sample containing the target agent), diluents (such as, phosphate buffers, or saline buffers), a PGM, and/or wash solutions (such as, Tris buffers, saline buffer, or distilled water).

Such kits can include other components, such as a buffer, a chart for correlating detected glucose level and amount of target agent present, the reagents needed to ultimately produce the glucose (such as ALP, antibodies, and starting materials) or combinations thereof. For example, the kit can include a vial containing one or more of the sensors disclosed herein and a separate vial containing a buffer (for example a lysis buffer).

Other kit embodiments include syringes, finger-prick devices, alcohol swabs, gauze squares, cotton balls, bandages, latex gloves, incubation trays with variable numbers of troughs, adhesive plate sealers, data reporting sheets, which may be useful for handling, collecting and/or processing a biological sample. Kits may also optionally contain implements useful for introducing samples onto a lateral flow device, including, for example, droppers, Dispo-pipettes, capillary tubes, rubber bulbs (e.g., for capillary tubes), and the like. Still other kit embodiments may include disposal means for discarding a used device and/or other items used with the device (such as patient samples, etc.). Such disposal means can include, without limitation, containers that are capable of containing leakage from discarded materials, such as plastic, metal or other impermeable bags, boxes or containers.

In some examples, a kit will include instructions for the use of a sensor or lateral flow device. The instructions may provide direction on how to apply sample to the sensor or device, the amount of time necessary or advisable to wait for results to develop, and details on how to read and interpret the results of the test. Such instructions may also include standards, such as standard tables, graphs, or pictures for comparison of the results of a test. These standards may optionally include the information necessary to quantify target analyte using the sensor or device, such as a standard curve relating amount of glucose detected to an amount of target analyte therefore present in the sample.

### Example 1

### Materials and Methods

This example describes the materials and methods used in Examples 2-7 below.

### Materials

Galactose-1-phosphate uridyltransferase (GALT) from galactose-adapted yeast, glucose-6-phosphate dehydrogenase (G6PD) from baker's yeast, phosphoglucomutase from rabbit muscle, alkaline phosphatase (ALP) from bovine intestinal mucosa, D-(+)-glucose, D-(+)-galactose, α-D-galactose-1-phosphate dipotassium salt pentahydrate, α-D-glucose-1-phosphate disodium salt hydrate, uridine-5′-diphosphoglucose disodium salt (UDP-glucose), β-nicotinamide adenine dinucleotide phosphate disodium salt (NADP⁺), 4-nitrophenyl phosphate diTris salt, human serum and other reagents for buffers and solvents were from Sigma-Aldrich, Inc. (St. Louis, Mo.). Bovine calf whole blood and lysed horse blood were from Lampire Biological Laboratories, Inc. (Pipersville, Pa.). The four brands of personal glucose meters (PGMs) used were Accu-Chek Avia, Free Style Lite, Bayer Breeze 2 and Medisense Optimum Xeed.

Buffers used: 0.1 M sodium carbonate buffer at pH 9.9; 0.2 M sodium phosphate buffer at pH 7.0; 0.05, 0.15, 0.3 and 1 M glycine buffers at pH 8.7; and 0.1 M citrate buffer at pH 7.5.

### GALT Activity Assays Using a Traditional Method

The measurement and calibration of GALT activity using a traditional method was carried out following a reported procedure.³² A mixture of 5 mL 50 mM glycine buffer pH 8.7 containing 1 mM EDTA, 0.2 mL 0.1 M MgCl₂, 0.1 mL 25 mM NADP⁺, 0.2 mL 80 mM galactose-1-phosphate, 0.02 mL 2000 U/mL phosphoglucomutase and 0.02 mL 700 U/mL glucose-6-phosphate dehydrogenase was used as the working reagent. For each assay, 80 μL working reagent, 2.5 μL GALT stock solution and 5 μL 22 mM UDP-glucose were mixed and then monitored at 340 nm in a UV-Vis spectrometer for 20 min. The GALT activities in the test solutions were calculated by the rate of absorbance enhancement using the equation: GALT activity (U/mL)=ΔA×10³/(6.22×2), where ΔA is the change of absorbance at 340 nm per min. The 6.22 is the extinction coefficient of NAPDH at 340 nm per mole/L. The production of one mole glucose-1-phosphate is responsible for the formation of 2 mole NAPDH from NAPD⁺.

### Hydrolysis of Glucose-1-Phosphate by ALP

To demonstrate that the hydrolysis of glucose-1-phosphate by ALP could proceed efficiently and that the glucose produced via the reaction could be quantified by commercially available PGMs, glucose-1-phosphate solutions were measured after hydrolysis by different concentrations of ALP using the Accu-Chek Avia PGM. The activity of the ALP stock solution (after 1/10000 dilution) purchased from Sigma-Aldrich was determined by monitoring the increase of absorbance at 405 nm induced by the hydrolysis of 4-nitrophenyl phosphate in 0.1 M sodium carbonate buffer at pH 9.9. One unit of enzyme represents the amount of ALP that can hydrolyze 1 μmol 4-nitrophenyl phosphate to 4-nitrophenol and phosphate within 1 min in the same buffer at 25° C. The experiments were conducted in a series of 100 μL 0.1 M sodium carbonate buffer solutions at pH 9.9 containing 11.5 mM glucose-1-phosphate and different concentrations of ALP. After one hour for the enzymatic reaction, 6 μL of 1 M NaH₂PO₄ was added to each 100 μL assay solution above and then the solutions were measured by the PGM. The NaH₂PO₄ addition neutralized the solutions, as basic solutions are not suitable for PGM measurement.

### Selectivity of Glucose Measurement by PGMs Over Galactose

As shown in FIG. 1B, galactose is produced from galactose-1-phosphate catalyzed by ALP even in the absence of GALT. Therefore, is galactose formed as the background during the measurement of glucose in the GALT activity assays using PGMs. There are reports demonstrating that some PGMs can successfully quantify the concentration of glucose without any interference from galactose depending on the enzymes and oxidants used for the manufacturing of the strips. To determine if the PGMs used herein could efficiently detect glucose with no signal response from galactose, four brands of PGMs (Accu-Chek Avia, Free Style Lite, Bayer Breeze 2 and Medisense Optimum Xeed) were used to measure 0.2 M sodium phosphate buffers at pH 7.0 containing 10 mM glucose or galactose.

### Quantification of GALT Activity in Buffers

To a 60 μL 0.15 M glycine buffer at pH 8.7 containing 9 mM UDP-glucose and 10 mM galactose-1-phosphate, 6 μL 0.1 M sodium citrate buffer at pH 7.5 containing concentrations of GALT as stocks was added and mixed to reach a final concentration of 0˜0.46 U/mL. The solution was left to stand at 25° C. for 40 min, and a mixture of 5 μL 1 M Na₂CO₃ and 3 μL 0.3 M glycine buffer at pH 8.7 containing 800 U/mL ALP was added (the final concentration of ALP was 32 U/mL, in large excess compared to GALT). After 18 min, 10 μL 1 M NaH₂PO₄ was added and the solution was measured by the PGMs.

### Quantification of GALT Activity in Human Serum

To 25 μL human serum spiked with different concentrations of GALT, 10 μL 1 M glycine buffer at pH 8.7, 15 μL 45 mM UDP-glucose in water and 15 μL 42 mM galactose-1-phosphate were added and mixed. The solution was kept at 25° C. for 10˜100 min, and a mixture of 5 μL 1 M Na₂CO₃ and 3.5 μL 0.3 M glycine buffer at pH 8.7 containing 1000 U/mL ALP was added (the final concentration of ALP was 47.6 U/mL, in large excess compared to GALT). After 10˜40 min, 10 μL 1 M NaH₂PO₄ was added and the solution was measured by the Bayer Breeze 2 PGM. Because the human serums were diluted by 3-fold during the test, the GALT activities in the samples in the final calibration curve were also tripled to represent the real concentration of GALT spiked into the serum samples.

### Quantification of GALT Activity in Animal Blood

To 25 μL calf blood (non-lysed) or horse blood (lysed) containing different concentrations of GALT, 10 μL 1 M glycine buffer at pH 8.7, 15 μL 45 mM UDP-glucose in water and 15 μL 42 mM galactose-1-phosphate were added and mixed. The solution was kept at 25° C. for 10˜80 min, and a mixture of 5 μL 1 M Na₂CO₃ and 3.5 μL 0.3 M glycine buffer at pH 8.7 containing 1000 U/mL ALP was added (the total concentration of ALP was 47.6 U/mL, in large excess compared to GALT). After 15 min, 10 μL 1 M NaH₂PO₄ was added and the solution was measured by the Bayer Breeze 2 PGM. Because the blood samples were diluted by 3-fold during the test, the GALT activities in the samples in the final calibration curve were also tripled to represent the real concentration of GALT in the blood samples.

### Example 2

### Conversion of Glucose-1-Phosphate to Glucose Using ALP

This example describes methods used to demonstrate that ALP can efficiently hydrolyze glucose-1-phosphate and transform the concentration of glucose-1-phosphate into that of glucose for signal readouts in PGMs, as shown in FIG. 1B.

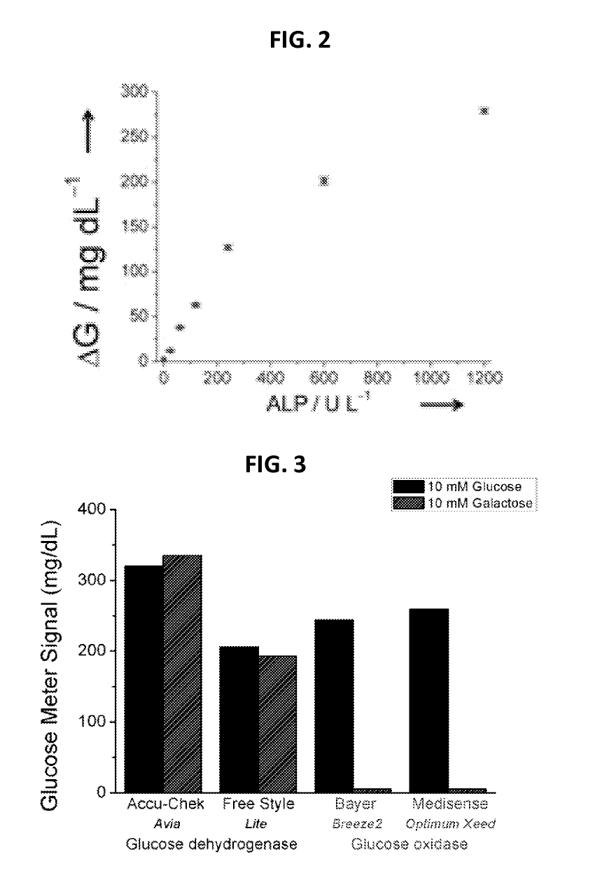

In sodium carbonate buffer (pH 9.9) solutions containing 11.5 mM glucose-1-phosphate, different amounts of ALP were added and the resulting solutions were measured by the PGM after 1 hour reaction. As shown in FIG. 2, more glucose is detected by the PGM in the presence of higher amounts of ALP activities in solution. This demonstrates that ALP can hydrolyze glucose-1-phosphate and can be used to correlate the detection of glucose and glucose-1-phosphate for the PGM to further quantify GALT activities.

### Example 3

### PGM Response to Glucose and Galactose

This example describes methods used to confirm the specificity of commercially available PGMs for glucose.

The GALT enzyme catalyzes the equilibrium conversion between galactose-1-phosphate and glucose-1-phosphate. Thus galactose and glucose are produced from galactose-1-phosphate and glucose-1-phosphate, respectively, after applying ALP to the reaction mixture (FIG. 1B). The chemical structures of galactose and glucose are almost the same except for the difference in the stereochemistry of C4. To quantify GALT activity accurately, PGMs ideally measure glucose while give little or no response to galactose. Otherwise, the test results would not be reliable and cause false positive results because large amounts of galactose are produced from galactose-1-phosphate by ALP-catalyzed hydrolysis even in the absence of GALT.

Four types of PGMs were evaluated for their ability to differentiate glucose and galactose by testing two neutral phosphate buffer solutions containing 10 mM galactose and glucose, respectively. As shown in FIG. 3, two (Bayer Breeze 2 and Medisense Optimum Xeed) of the four PGMs had selective signal responses to glucose over galactose. The results are in accordance with a published study that glucose meters based on glucose oxidase can measure glucose with no interference from other sugars, while those based on glucose dehydrogenase/pyrroloquinolinequinone GDH/PQQ cannot.³³

Therefore, the Bayer Breeze 2 glucose meter was used in the Examples below to measure GALT activities in different samples.

### Example 4

### PGM Measurement of GALT Activity and Comparison to Traditional Methods

This example describes methods used to measure galactose-1-phosphate uridylyltransferase (GALT) activity using commercially available PGMs.

Since ALP and the PGMs can successfully hydrolyze glucose-1-phosphate to glucose and selectively measure glucose over galactose, the method was used to quantify GALT activities using PGMs and to compare such measurements with the traditional method based on spectroscopic measurement of NADPH production.

In a 0.15 M glycine buffer solution at pH 8.7 containing the substrates (UDP-glucose and galactose-1-phosphate), different concentrations of GALT were added (0 to 460 U/L). After the enzymatic reaction catalyzed by GALT, the product glucose-1-phosphate was further hydrolyzed by the subsequently added ALP. ALP was added in much more excess (32000 U/L) than GALT (<500 U/L)) to ensure complete hydrolysis of the phosphorylated sugars (glucose-1-phosphate and galactose-1-phosphate) in a short time, and to quench the GALT-catalyzed reaction., Finally, the solution was neutralized and measured by the PGMs, and GALT activity was calculated based on the glucose signal readouts.

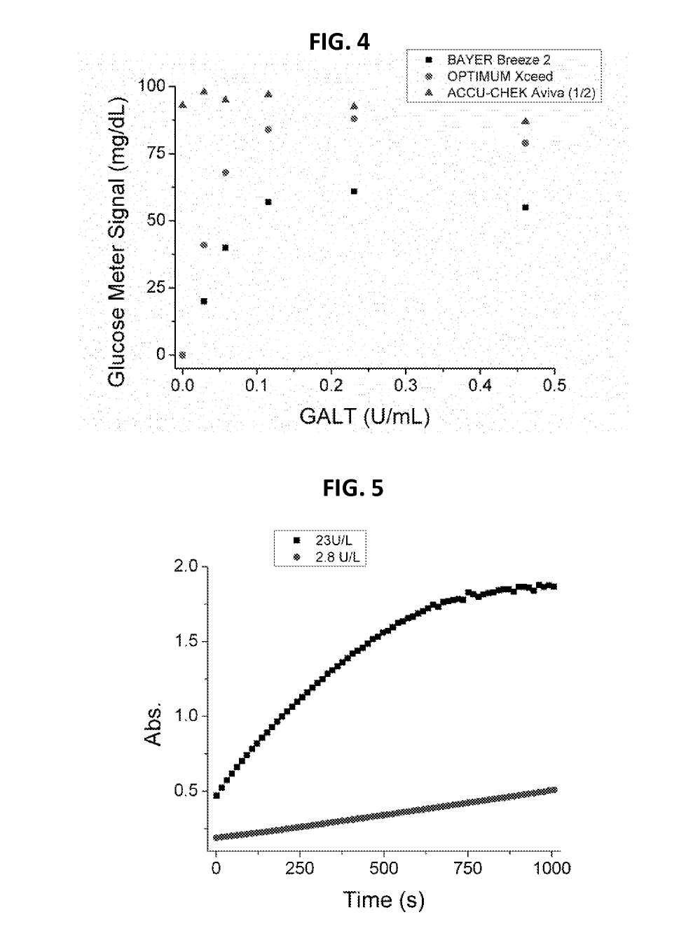

As illustrated in FIG. 4, higher signal readouts in PGMs (selective to glucose, Bayer (squares) and Optimum (circles)) were observed with more GALT activities. The difference in the signal readouts of the two PGMs when measuring the same samples with identical GALT activities is likely due to the different manufacturing of the PGMs, which were calibrated for tests in blood but not in buffer. In contrast, if a PGM with no selectivity to glucose over galactose (Accu-chek, triangles) was used, the signal readouts showed little difference even if the samples containing different amounts of GALT were measured by the PGM, because the total concentration of galactose-1-phosphate and glucose-1-phosphate was constant during the GALT-catalyzed reaction and both of the two phosphorylated sugar could be efficiently hydrolyzed by ALP and measured by the PGM.

To compare with the measurements of GALT activities by PGM-based method and traditional methods, a traditional spectroscopic method for GALT assays was also performed.³² UPD-glucose, galactose-1-phosphate, GALT, phosphoglucomutase, NADP and glucose-6-phosphate dehydrogenase were mixed in 0.05 M glycine buffer (pH 8.7) and monitored continuously in a UV-Vis spectrometer. The absorbance enhancement at 340 nm due to the formation of NADPH from NADP was used to calculate the amount of glucose-1-phosphate produced in the reaction to assess GALT activity.

The results (FIG. 5) were used to calibrate the GALT activity used herein. Although the traditional method efficiently measured GALT activities in the buffer solutions that were completely colorless and transparent, they encountered significant interference when the solutions were colored or turbid, for example, blood or serum samples (unless pre-separations steps were carried out to remove the serum proteins and hemoglobin). However, the disclosed new method based on PGMs was completely independent of the physical appearances of the samples. Moreover, the traditional method required phosphoglucomutase, NADP and glucose-6-phosphate dehydrogenase to transform glucose-1-phosphate into the detectable absorption of NADPH, while the new method only requires ALP as the enzyme to convert glucose-1-phosphate into PGM-detectable glucose.

### Example 5

### PGM Measurement of GALT Activity in Serum Samples

This example describes methods used to measure galactose-1-phosphate uridylyltransferase (GALT) activity in serum samples using commercially available PGMs.

As shown above, the disclosed method based on PGMs is advantageous over traditional methods not only because quantitative detection was achieved with cheap and portable PGMs instead of laboratory-based spectrometers, but also due to the resistance to the intrinsic physical properties of the samples, such as the red color of blood. Therefore, the method was used to measure GALT activities in human serum and animal blood samples to demonstrate the tolerance of sample matrix. Human serum samples were spiked with different concentrations of GALT and then mixed with 1 M glycine buffers (pH 8.7) containing UDP-glucose and galactose-1-phosphate. After reaction, the mixture was treated with large excess amount of ALP (>32 U/mL) to quench the reaction and hydrolyze glucose-1-phosphate into PGM-detectable glucose. The neutralized solutions were measured by the PGM.

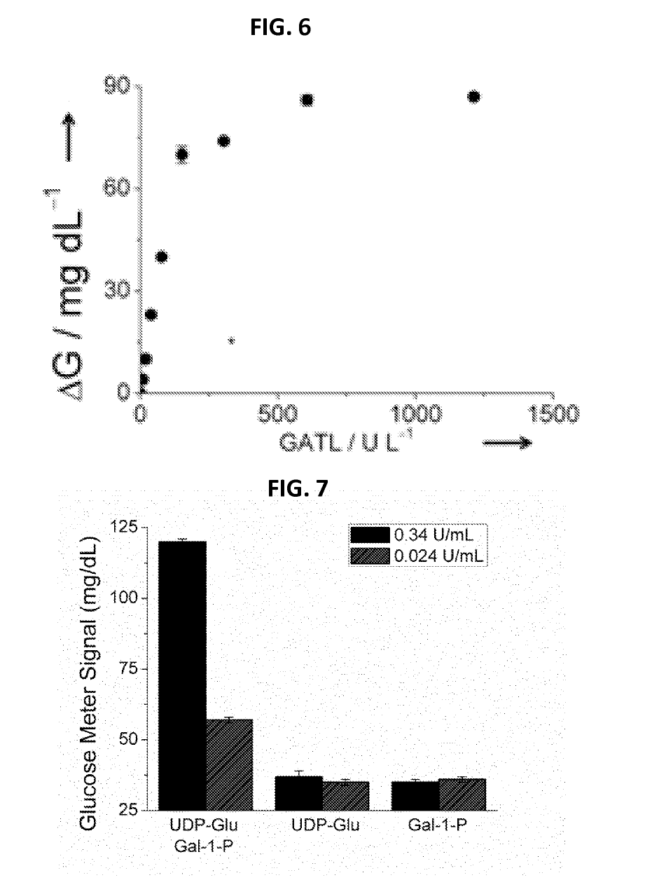

As shown in FIG. 6, the signal readouts from the PGM successfully quantified the GALT activities spiked into the human serum samples, without the interference from the complicated serum proteins and other components. No pre-separation step to remove serum proteins or other components from the serum was required. The method was very sensitive to GALT activity, achieving a detection limit (based on 3σ_{b}/slop) of 6 U/L in human serum. This detection is lower than the threshold of ˜20 U/L (estimated from 2.5-3.5 U/g Hb) obtained from classic galactosemia diagnosis methods,²⁷ demonstrating that the disclosed method can be used to quantify GALT activities under the clinically relevant range.

Glucose is present in human serum, and it can be detected from the signal readouts in PGMs for the assessment of GALT activities to avoid the fluctuation of glucose concentrations in human serums from different sources. To measure the original glucose present in the human serum samples, similar tests were carried out as above, but with added UDP-glucose or galactose-1-phosphate.

As shown in FIG. 7, no glucose signal enhancement was observed when either UDP-glucose or galactose-1-phosphate was absent, regardless of the concentration of GALT spiked in the samples (FIG. 7, middle and right columns), indicating that no GALT-catalyzed reaction occurred in these two “controls”, otherwise the signals would be GALT-dependent as the case when both substrate were present (FIG. 7, left column). In addition, the glucose signals observed by measuring human serum without added GALT using this method did not change with increasing reaction time, indicating the human serum used herein had almost no detectable GALT activity. The lack of naive GALT activity in the human serum samples may be ascribed to the loss of GALT activity when the serum was extracted from blood or stored by the vendor. By determining the original glucose signal of serum samples as measured above, the GALT activity assays using this PGM-based method are independent of the original serum glucose.

### Example 6

### Effect of GALT- and ALP-Reaction Time on PGM Measurement of GALT Activity

This example describes methods used to determine the effect of both GALT- and ALP-reaction times on the signal detected when measuring galactose-1-phosphate uridylyltransferase (GALT) activity using commercially available PGMs.

The GALT activity measurement in the disclosed method involves two reaction steps: (1) GALT-catalyzed transformation of UDP-glucose and galactose-1-phosphate into UDP-galactose and glucose-1-phosphate; (2) ALP-catalyzed conversion of PGM-inert glucose-1-phosphate into PGM-detectable glucose, along with the conversion of galactose-1-phosphate into galactose (FIGS. 1A and 1B). The effect of reaction time during the two steps on the final signal readouts measured by the PGM was studied to determine the kinetics.

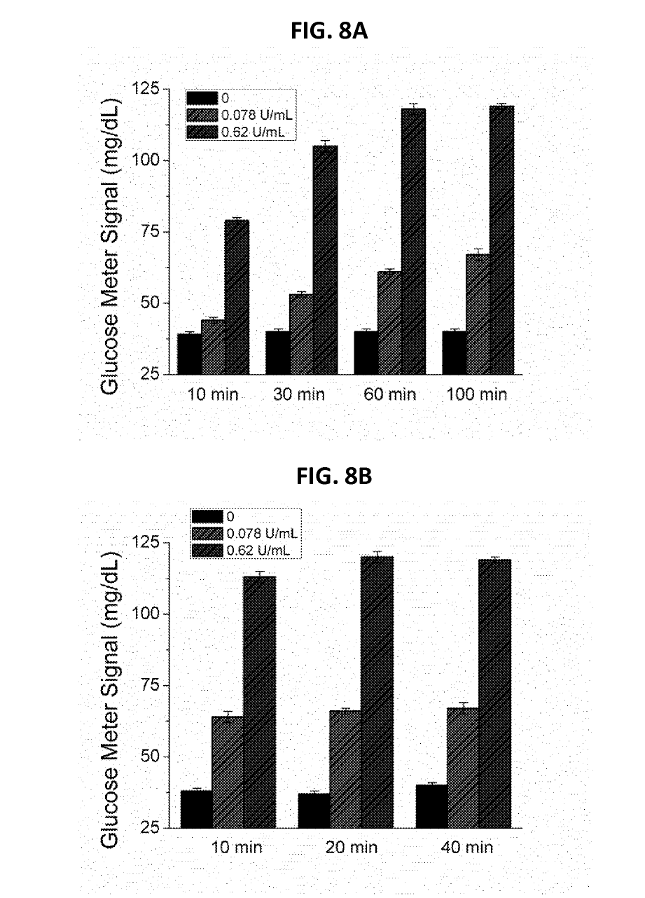

As shown in FIG. 8A, in the presence of high GALT activity (62 U/L), the production of PGM-detectable glucose was very fast, with large (˜40 mg/dL) signal enhancement over the control (0 U/mL) in just 10 min; while at lower GALT activity (78 U/L), the reaction was slower but still showed detectable (˜5 mg/dL) signal change compared with the control in 10 min. The change became larger with longer reaction time.

In contrast, the ALP-catalyzed reaction in the second step was much faster (<1 min as estimated by the activity), because ALP (32000 U/L) was added in much excess compared to GALT (<1500 U/L) present in the samples. The tests with 10, 20 or 40 minutes of ALP reaction time gave nearly the same glucose signal response in the PGM (FIG. 8B).

Thus, the ALP-catalyzed hydrolysis of glucose-1-phosphate and galactose-1-phosphate is so fast that the GALT-catalyzed reaction could be considered quenched soon after the addition of ALP. Therefore, the total time for a GALT activity test can be completed in less than 30 minutes using the disclosed methods.

### Example 7

### Measurement of GALT in Blood Samples Using PGM

This example describes methods used to measure GALT activity in blood samples using commercially available PGMs.

A nonlysed calf blood sample was tested using the disclosed method by mixing the blood with glycine buffer solutions containing UDP-glucose and galactose-1-phosphate and then treated with large excess ALP. However, no detectable signal in the PGM was observed for either the calf blood sample itself or after the GALT-catalyzed reaction, indicating that the calf blood had very low levels of glucose or GALT activity. The low GALT activity in the blood sample may be partially because it was not lysed so that the GALT inside red blood cells could not contact with the substrates. Therefore, the blood sample was spiked with external GALT to demonstrate the method could tolerate the sample matrix of the calf blood.

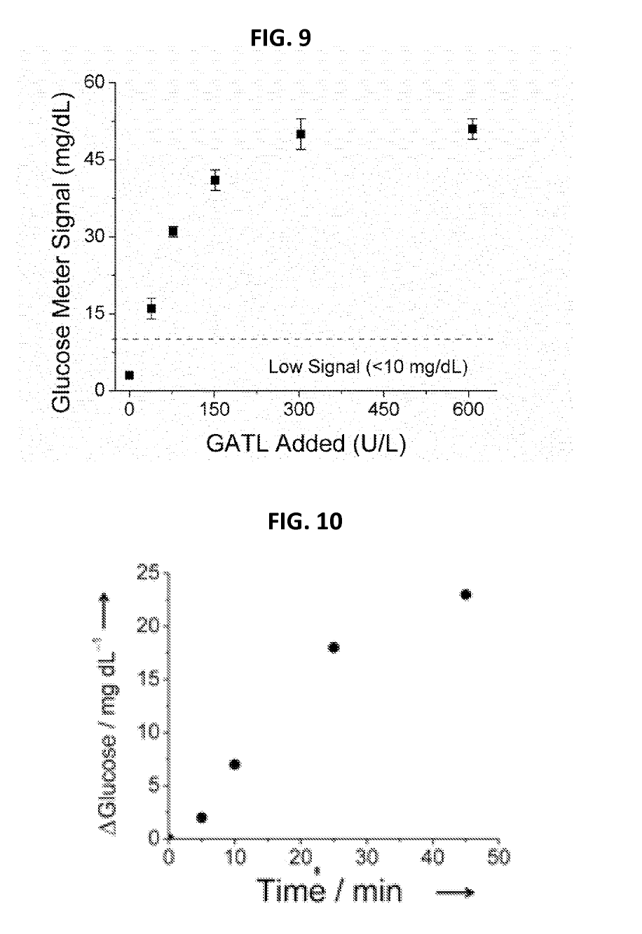

As shown in FIG. 9, in the presence of increasing concentrations of GALT added to the calf blood, greater glucose signals were observed in the PGM. The result confirmed that the method is resistant to the components in the calf blood.

Furthermore, a lysed horse blood sample was also tested. In contrast to the calf blood sample, the lysed horse blood was positive in GALT activity. As shown in FIG. 10, without adding any extrinsic GALT, the horse blood catalyzed the production of glucose from UDP-glucose and galactose-1-phosphate with the aid of ALP. In addition, more glucose was detected by the PGM after longer time of reaction. The original GALT activity of the horse blood was high because detectable glucose production could be observed even after 5 minutes of reaction time.

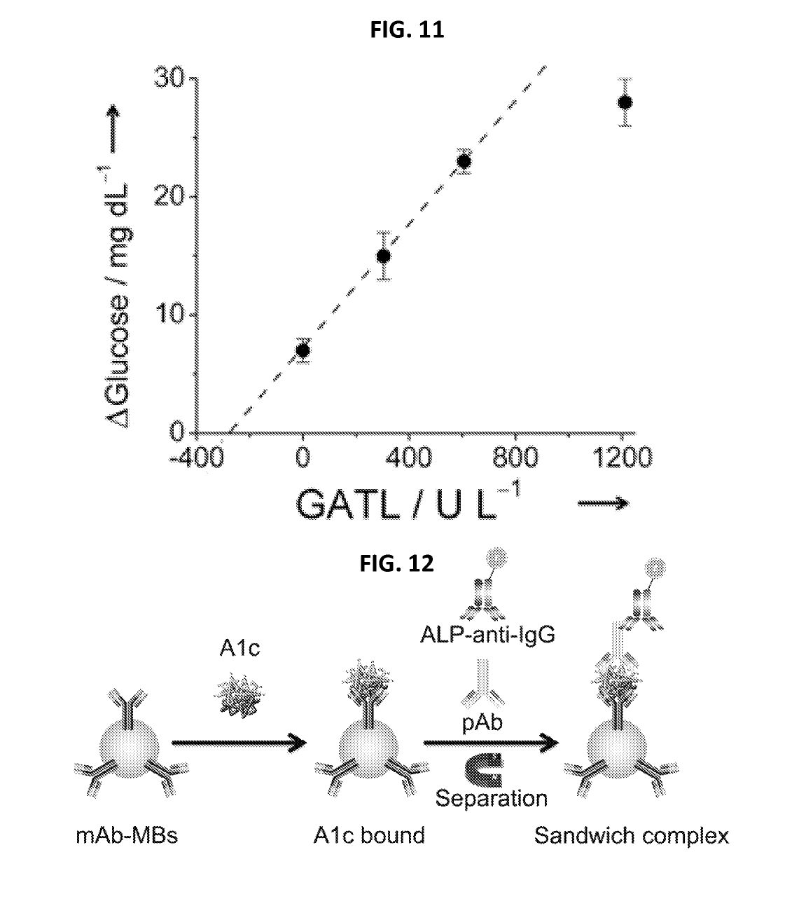

The horse blood was also spiked with increasing amounts of GALT and found more glucose production after a 10 minute reaction (FIG. 11). This permitted estimation of the original GALT activity in the horse blood to be about 276 U/L based on the standard addition method.

In summary, a new methodology using personal glucose meters (PGMs) for quantitative measurement of galactose-1-phosphate uridylyltransferase (GALT) activities in samples (such as serum and blood samples) is provided. In contrast to traditional GALT assays that involve two additional enzymes, spectroscopic measurement and hemoglobin removal procedures, the new method requires only one enzyme (alkaline phosphatase) to generate the signal-responsible product (glucose), and the GALT activity measurement is accomplished by the PGMs that are not interfered by the intrinsic color or turbidity of the blood or serum samples. Thus, sample pre-treatment is not required. In human serum, the GALT activity assays using the disclosed method achieved a 6 U/L sensitivity below the threshold (about 20 U/L) for diagnosis in human serum, and can also be successfully applied to detect GALT in blood samples. Furthermore, the GALT measurements in blood samples were also successful and could be used to assess GALT activity for classic galactosemia diagnosis in a point-of-care diagnosis by the general public.

### Example 8

### Materials and Methods for Hb A1c Detection

This example describes the materials and methods used for Example 9.

### Materials

Epoxyl magnetic-bead-antibody conjugation kit (Dynabeads M-270) was from Invitrogen Inc. (Carlsbad, Calif.). Amicon centrifugal filters were from Millipore Inc. (Billerica, Mass.). Human hemoglobin A1c (Hb A1c) protein (ab82273), mouse monoclonal antibody (mAb) to hemoglobin A1c (ab33847), rabbit polyclonal antibody (pAb) to hemoglobin A1c (ab31152) and mouse monoclonal secondary antibody to rabbit IgG conjugated with alkaline phosphatase (ab99701) were from Abcam Inc. (Cambridge, Mass.). Human hemoglobin (Hb), bovine serum albumin (BSA), glucose-1-phosphate and other chemicals for buffers and solvents were from Sigma-Aldrich, Inc. (St. Louis, Mo.).

Buffers used: 0.1 M sodium carbonate buffer at pH 9.9; 0.2 M sodium phosphate buffer at pH 7.0; 0.05, 0.15, 0.3 and 1 M glycine buffers at pH 8.7; 0.1 M citrate buffer at pH 7.5; 0.1 M sodium phosphate pH 7.0, 0.1 M NaCl, 1 g/L BSA.

### Antibody Conjugation to Expoxyl Magnetic Beads

The antibody conjugation was according to the protocol provided with the Invitrogen expoxy MB-antibody conjugation kit. Solutions C1, C2, HB, LB and SB were in the conjugation kit and were used directly without any treatment.

A portion of 8 mg Dynabeads M-270 magnetic beads (MBs) was washed by 1 mL C1 solution and the supernatant was removed by a magnet. Then, the residue was dispersed in a 0.5 mL mixed solution of C1 and C2 (v/v=1:1) and added with 50 μg mouse monoclonal antibody to hemoglobin A1c (ab33847). The solution was then kept on a roller at room temperature overnight. After that, the supernatant was removed by a magnet, and the solid residue was washed by 0.8 mL solution HB. This washing step was then repeated twice using 0.8 mL solution LB and SB instead of HB, respectively. Then, the solid residue was re-dispersed in 0.8 mL solution SB and stood on a roller for 15 min before the removal of supernatant by a magnet. Finally, the solid residue was dispersed in 0.5 mL solution SB and stored at 4° C.

### Procedures for the Hemoglobin A1c Assay Using a PGM

A portion of 100 μL 4 mg/mL (0.4 mg) antibody conjugated MBs was used for one test. The supernatant was removed by a magnet, and then 100 μL samples containing different concentrations of hemoglobin A1c (Hb A1c) in the assay buffer (0.1 M sodium phosphate pH 7.0, 0.1 M NaCl, 1 g/L BSA) were added to the MB residue and mixed. After 30 min at room temperature, the supernatant was removed by a magnet and the MB residue was washed by the assay buffer for 3 times. Then, 100 μL 25 mg/L rabbit polyclonal antibody to hemoglobin A1c (ab31152) in the assay buffer was added to the MB residue and mixed at room temperature for 30 min. After removal of supernatant and twice wash using the assay buffer, 100 μL 1/100 diluted of mouse monoclonal secondary antibody to rabbit IgG conjugated with ALP (ab99701) was added to the MB residue and reacted at room temperature for 30 min, followed by 3 times wash using the assay buffer and then dispersed in 100 μL 0.1 M sodium carbonate buffer pH 9.9 containing 3 g/L glucose-1-phosphate for 2.5 h. Finally, 6 μL 1 M NaH₂PO₄ was added to the solution to neutralize the pH and measured by a PGM.

### Example 9

### Hb A1c Detection for Diabetes Diagnosis

This example describes methods of using ALP-conjugated antibodies to detect HbAc1.

To demonstrate that ALP-conjugated antibodies could be used in immunoassays for PGM-based quantification of non-glucose analytes, glycated human hemoglobin (Hb A1c) was chosen as an example. Hb A1c is a glycated form of hemoglobin yielded non-enzymatically by the long-term contact of blood glucose and hemoglobin. It is used as an alternative indication of diabetes in addition to blood glucose levels. Compared to blood glucose tests that reveal the instant glucose levels in blood, Hb A1c tests give the average blood glucose over prolong time periods. Normal blood glucose induces the formation of Hb A1c as about 6.5% of the total Hb in human blood, while in diabetes patients the level is generally higher. Type I and II diabetes patients are recommended to take the Hb A1c tests every 4 and 2 times, respectively, to monitor the disease status. For the public, Hb A1c tests may be more reliable diagnosis of diabetes risk than blood glucose tests because the latter may be affected by many factors such as the digested food, medical treatment and health status of the person a short time prior to the tests. Hb A1c tests are available in hospitals and medical centers. For more inexpensive and convenient tests, commercial kits and meters for Hb A1c are also available for the public.

As shown in FIG. 12, the PGM-based Hb A1c assay was based on the sandwich complex formation by Hb A1c protein and two Hb A1c antibodies. In the presence of Hb A1c, the mAb covalently immobilized on the surface of MBs could bind and capture the Hb A1c from the sample solutions. Subsequently, the pAb that binds to a different site of Hb A1c compared to the mAb was added and formed the sandwich complex. Further addition of a secondary antibody (conjugated with ALP, abbreviated as ALP-anti-IgG) specific to the IgG of the pAb could bind the pAb on the MBs and label the MBs with ALP, which can catalyze the hydrolysis of glucose-1-phosphate to produce glucose (FIG. 1B) for the signal readout in PGMs. In contrast, in the absence of Hb A1c, the sandwich complex does not form, so that little or no signal in PGMs was detected, because glucose-1-phosphate is inert to PGMs.

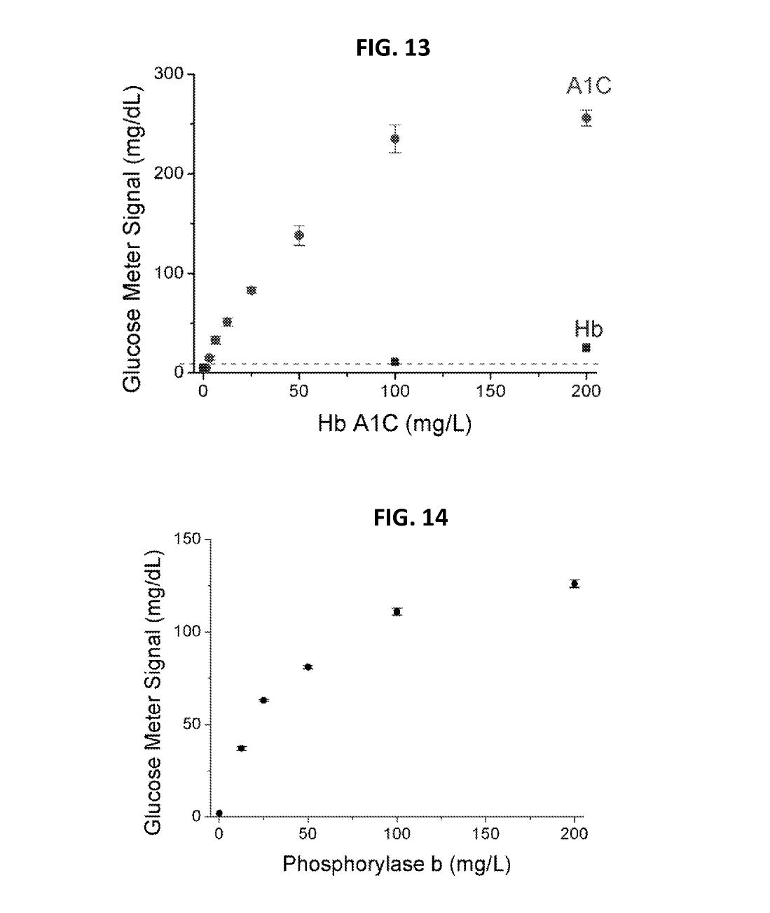

Using the methodology shown in FIG. 12, Hb A1c in solution was quantified (FIG. 13). In an assay solution (0.1 M sodium phosphate pH 7.0, 0.1 M NaCl, 1 g/L BSA) containing increasing concentrations of Hb A1c (0˜200 mg/L), the glucose signals detected by the PGMs were increased accordingly, with an approximately linear relationship up to 100 mg/L and reaching the saturation, at which concentration almost all the binding sites on the mAb-MBs for Hb A1c should be occupied by the analyte so that more Hb A1c could not further enhance the signal. The detection limit was about 3 mg/L, which is sufficient to monitor the Hb A1c in blood with concentrations as high as 100 g/L (about 7% Hb A1c of total Hb) after extensive dilutions.

### Example 10

### Measurement of Glycogen Phosphorylase (GP) Activity

This example describes methods used to measure GP activity in a sample using a PGM.

GP and 5′-AMP convert glycogen to glucose-1-phosphate, which can be converted to glucose using AP as shown below.

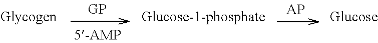

Experiments were performed generally as described in Example 1 for GALT in solution. Assay buffer containing 100 uL 0.1 M sodium phosphate buffer pH 7.0, 5 g/L glycogen, 0.5 mM MgAc₂ and 0.1 mM 5′-AMP was incubated for 30 min with different amounts of GP (phosphorylase b from rabbit, 27 U/mg). Then, 6 μL 1 M Na₂CO₃, 4000 U/L AP, and reacted for 15 min. Then, 6 μL 1M NaH₂PO₄ was added and the glucose produced measure using a glucose meter.

As shown in FIG. 14, the method successfully measured GP activity.

### Example 11

### Lateral Flow Device that Includes ALP

This example describes an exemplary lateral flow device that can be used to detect a target agent in a test sample using the sensors disclosed herein. One skilled in the art will appreciate that similar devices can be generated by attaching other starting products and by testing for the presence other enzymes that produce glucose-1-phosphate. For example, the lateral flow device described in this example can detect GALT; however, the sensor may use other starting products to detect different target enzymes.

FIG. 15 shows a lateral flow device that can be read by a PGM for detecting a broad range of non-glucose targets in many different samples, using a lateral flow device containing one or more starting products that can be converted to glucose-1-phosphate, and ALP. ALP can convert glucose-1-phosphate into glucose.

As shown in FIG. 15, the lateral flow device contains wicking or sample pad, multiple reagent/conjugation pads, multiple reaction pads/membranes, and an absorption pad. The sample containing or suspected of containing one or more target agents is applied to the sample pad. If desired, liquid can be added to the sample, or the sample can be concentrated, before applying it to the sample pad. The sample pad ensures a controllable (unilateral) flow of the sample. The sample migrates from the bottom to the top of the lateral flow device following the indicated flow direction in FIG. 15 because of capillary force. When the sample reaches the first reagent pad, which contains the one or more starting products that are reacted upon by GALT (galactose-1-phosphate and UDP-glucose), it transports these to the first reaction pad. Here, if GALT is present in the sample, it will react with the galactose-1-phosphate and UDP-glucose to generate glucose-1-phosphate (and UDP-galactose). The resulting glucose-1-phosphate moves with the flow to the second reagent pad, containing ALP and buffer components. The ALP and glucose-1-phosphate moves with the flow to the second reaction pad, where the ALP converts the glucose-1-phosphate to glucose. The resulting glucose moves with the flow to the third reagent pad where which contains pH neutralizing agents (for example an agent than can neutralize the pH from basic to neutral) and finally reaches the absorption pad, where it is then detected by a PGM.

The amount of glucose detected by the PGM is proportional to the amount of GALT in the test sample. This permits quantification of the GALT by the read-out of glucose meter. The original glucose concentration in the sample can be subtracted from the result if desired.

### Example 12

### Lateral Flow Device that Includes ALP-Conjugated Antibodies

This example describes an exemplary lateral flow device that can be used to detect a target agent in a test sample using the sensors disclosed herein. One skilled in the art will appreciate that similar devices can be generated by using antibodies specific for other targets. For example, the lateral flow device described in this example can detect Hb A1c; however, the sensor may use other antibodies to detect different target agents.

FIG. 17 shows a lateral flow device that can be read by a PGM for detecting a broad range of non-glucose targets in many different samples, using a lateral flow device containing one or more ALP-conjugated antibodies. ALP can convert glucose-1-phosphate into glucose.

As shown in FIG. 17, the lateral flow device contains wicking or sample pad, multiple reagent/conjugation pads (which can be interspersed with membranes/reaction pads, not shown) and an absorption pad. The sample containing or suspected of containing one or more target agents is applied to the sample pad. If desired, liquid can be added to the sample, or the sample can be concentrated, before applying it to the sample pad. The sample pad ensures a controllable (unilateral) flow of the sample. The sample migrates from the bottom to the top of the lateral flow device following the indicated flow direction in FIG. 17 because of capillary force. When the sample reaches the reagent pad, which contains biotin-anti HbA1c antibodies, streptavidin, and biotin-ALP conjugate, HbA1c present in the sample binds to the biotin-anti HbA1c antibodies. The streptavidin binds to the biotin, and the biotin-ALP conjugate binds to the streptavidin. Alternatively, a biotin-ALP-streptavidin-biotin-anti HbA1c antibody complex is present on the lateral flow device, wherein the biotin-anti HbA1c antibody of the complex binds to the target. The resulting target-biotin-anti HbA1c antibody-streptavidin-biotin-ALP complex moves with the flow to the second reagent pad containing immobilized anti Hb antibody, which it is captured to the solid phase rather than remaining in the mobile phase (flow solution). Materials in the mobile phase move to the area containing glucose-1-phosphate. If ALP is present in the mobile phase, it will convert the glucose-1-phosphate to glucose, which flows to the absorption pad where it is then detected by a PGM. The more HbA1c present in the sample, the more ALP will be captured to the solid phase rather than remaining in the mobile phase (flow solution) and the less glucose signal will be detected.

The amount of glucose detected by the PGM is inversely proportional to the amount of HbA1c in the test sample. This permits quantification of the HbA1c by the read-out of glucose meter. The original glucose concentration in the sample can be subtracted from the result if desired.

### REFERENCES

(1) Daar et al., Nat. Genet. 2002, 32, 229-232.
(2) Giljohann, D. A.; Mirkin, C. A. Nature 2009, 462, 461-464.
(3) Wang et al., Angew. Chem., Int. Ed. 2009, 48, 856-870.
(4) Nolan, E. M.; Lippard, S. J. Chem. Rev. 2008, 108, 3443-3480.
(5) Que, E. L.; Domaille, D. W.; Chang, C. J. Chem. Rev. 2008, 108, 1517-1549.
(6) Chen, P.; He, C. Curr. Opin. Chem. Biol. 2008, 12, 214-221.
(7) Zhao et al., Angew. Chem., Int. Ed. 2008, 47, 6330-6337.
(8) Willner et al., Chem. Soc. Rev. 2008, 37, 1153-1165.
(9) Lubin, A. A.; Plaxco, K. W. Acc. Chem. Res. 2010, 43, 496-505.
(10) Cho et al., Annu. Rev. Anal. Chem. 2009, 2, 241-264.
(11) Li et al., Acc. Chem. Res. 2010, 43, 631-641.
(12) Fan et al., Trends Biotechnol. 2005, 23, 186-192.
(13) Zhang et al., J. Am. Chem. Soc. 2007, 129, 15448-15449.
(14) Wu et al., J. Am. Chem. Soc. 2009, 131, 12325-12332.
(15) Li, T.; Dong, S.; Wang, E. J. Am. Chem. Soc. 2010, 132, 13156-13157.
(16) Zhou et al., J. Am. Chem. Soc. 2010, 132, 2172-2174.
(17) Liu, J.; Cao, Z.; Lu, Y. Chem. Rev. 2009, 109, 1948-1998.
(18) Xiang, Y.; Lu, Y. Nat. Chem. 2011, 3, 697-703.
(19) Bosch, A. M. J. Inherit. Metab. Dis. 2006, 29, 516-525.
(20) Freer et al., Clin. Chem. 2010, 56, 437-444.
(21) Honeyman et al., Arch. Dis. Child. 1993, 69, 339-341.
(22) Bosch et al., Hum. Mutat. 2005, 25, 502-502.
(23) Beutler, E.; Baluda, M. C. J. Lab. Clin. Med. 1966, 68, 137-141.
(24) Fujimoto et al., Clin. Chem. 2000, 46, 806-810.
(25) Fujimura, Y.; Ishii, S.; Kawamura, M.; Naruse, H. Anal. Biochem. 1981, 117, 187-195.
(26) Jinks, D. C.; Vollmer, D.; Orfanos, A.; Guthrie, R. Clin. Biochem. 1987, 20, 353-357.
(27) www.state.nj.us/health/fhs/nbs/documents/galactosemia_prof.pdf.
(28) Clark, L. C.; Lyons, C. Ann. NY Acad. Sci. 1962, 102, 29-45.
(29) Montagnana et al., Clin. Chim. Acta 2009, 402, 7-13.
(30) Carroll, A. E.; Marrero, D. G.; Downs, S. M. Diabetes Technol. Ther. 2007, 9, 158-164.
(31) Coleman, J. E. Annu. Rev. Biophys. Biomolec. Struct. 1992, 21, 441-483.
(32) Pesce et al., Clin. Chem. 1977, 23, 1711-1717.
(33) Schleis, T. G. Pharmacotherapy 2007, 27, 1313-1321.

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

1. A method for detecting a target enzyme activity, comprising:
contacting a test sample with one or more starting products, wherein at least one of the starting products can be converted to glucose-1-phosphate by a target enzyme, comprises an enzyme involved in the production of glucose-1-phosphate, or combinations thereof;
allowing the target enzyme to convert the at least one starting product to glucose-1-phosphate;
contacting the glucose-1-phosphate with alkaline phosphatase (ALP);
allowing the ALP to convert the glucose-1-phosphate to glucose;
detecting the glucose with a personal glucose meter (PGM); and
determining the target enzyme activity by correlating the glucose detected.

2. (canceled)

3. The method of claim 1, wherein the test sample is a blood sample or fraction thereof or a tumor sample.

4. The method of claim 1, wherein the test sample is obtained from a subject, and the method further comprises determining that the subject has a disease based on the target enzyme activity detected.

5. (canceled)

6. The method of claim 1, wherein the method further comprises determining the target enzyme activity in a normal control sample, and comparing the target enzyme activity in the test sample and the normal control sample.

7. A method for detecting a target agent, comprising:
(a) contacting a test sample with a first antibody that specifically binds to the target agent, wherein the first antibody comprises ALP;
allowing the ALP-first antibody and the target agent to bind, thereby forming an ALP-first antibody-target agent complex;
contacting the ALP-first antibody-target agent complex with a second antibody that specifically binds to the target agent, wherein the second antibody is immobilized;
allowing the second antibody and the target agent of the first antibody-target agent complex to bind, thereby forming an immobilized ALP-first antibody-target agent-second antibody complex;
contacting non-immobilized agents with glucose-1-phosphate under conditions that permit ALP to convert the glucose-1-phosphate to glucose;
detecting the glucose with a personal glucose meter (PGM); and
determining that the target agent is present in the sample by correlating the glucose detected; or
(b) contacting a test sample with an ALP conjugated to a functional molecule, an agent that specifically binds to the functional molecule, and first antibody that specifically binds to the target agent, wherein the first antibody is conjugated to the functional molecule;
allowing the first antibody and the target agent to bind, the streptavidin to bind to the functional molecule—first antibody and to the functional molecule—ALP conjugate, thereby forming a first complex of functional molecule first antibody-target agent—agent that specifically binds to the functional molecule—functional molecule—ALP;
contacting the first complex with a second antibody that specifically binds to the target agent, wherein the second antibody is immobilized;
allowing the second antibody and the target agent of the first complex to bind, thereby forming a second complex of functional molecule first antibody-target agent—agent that specifically binds to the functional molecule—functional molecule—ALP-second antibody complex;
allowing non-immobilized ALP to interact with glucose-1-phosphate;
allowing the non-immobilized ALP to convert the glucose-1-phosphate to glucose;
detecting the glucose with a personal glucose meter (PGM); and
determining that the target agent is present in the sample by correlating the glucose detected, wherein the amount of target agent present in the sample is inversely proportional to the amount of glucose detected; or
(c) contacting a test sample with a target-specific binding agent-solid substrate complex, and with an alkaline-phosphate-target conjugate (ALP conjugate), wherein the target-specific binding agent specifically binds to the target;
allowing the target-specific binding agent-solid substrate complex to bind to the target and to the ALP conjugate, thereby forming a target-specific binding agent-solid substrate complex-target complex if the target is present in the test sample or thereby forming a target-specific binding agent-solid substrate complex-ALP conjugate complex if the target is not present in the test sample;
optionally separating the target-specific binding agent-solid substrate complex-target agent complex or the target-specific binding agent-solid substrate complex-ALP conjugate complex from a solution in which the target-specific binding agent-solid substrate complex-target agent complex or target-specific binding agent-solid substrate complex-ALP conjugate complex is present;
contacting the target-specific binding agent-solid substrate complex-target agent complex, the target-specific binding agent-solid substrate complex-ALP conjugate complex, the solution which contained the target-specific binding agent-solid substrate complex-target agent complex or the solution which contained the target-specific binding agent-solid substrate complex-ALP conjugate complex with glucose-1-phosphate under conditions that permit ALP to convert the glucose-1-phosphate to glucose;
detecting the glucose with a personal glucose meter (PGM); and
determining that the target is present in the sample by correlating the glucose detected; or
(d) contacting a test sample with a target-solid substrate complex, and with an alkaline-phosphate-target-specific antibody conjugate (ALP conjugate);
allowing the target to bind to the ALP conjugate, thereby forming a target-ALP conjugate complex if the target is present in the test sample or allowing the ALP conjugate to bind to the target-solid substrate complex, thereby forming a target-solid substrate-ALP conjugate complex if the target is not present in the test sample;
optionally separating the target-solid substrate complex from a solution in which the target-ALP conjugate complex is present or separating the target-solid substrate-ALP conjugate complex from a solution in which the target-solid substrate-ALP conjugate complex is present;
contacting the target-solid substrate complex, the solution containing the target-ALP conjugate complex, the solution which contained the target-solid substrate-ALP conjugate complex or the target-solid substrate-ALP conjugate complex with glucose-1-phosphate under conditions that permit ALP to convert the glucose-1-phosphate to glucose;
detecting the glucose with a personal glucose meter (PGM); and
determining that the target is present in the sample by correlating the glucose detected; or
(e) contacting a test sample with an antibody-solid substrate complex, wherein the antibody specifically binds to the target;
allowing the antibody-solid substrate complex to bind to the target, thereby forming an antibody-solid substrate complex-target complex if the target is present in the test sample;
contacting the antibody-solid substrate complex-target agent complex with an antibody specific for the target, thereby forming an antibody-solid substrate complex-target agent-antibody complex;
contacting the antibody-solid substrate complex-target agent-antibody complex with a ALP-labeled antibody, thereby forming an antibody-solid substrate complex-target agent-antibody-ALP-antibody complex;
optionally separating the antibody-solid substrate complex-target agent-antibody-ALP-antibody complex from a solution in which it is present;
contacting the antibody-solid substrate complex-target agent-antibody-ALP-antibody complex with glucose-1-phosphate under conditions that permit ALP to convert the glucose-1-phosphate to glucose;
detecting the glucose with a personal glucose meter (PGM); and
determining that the target is present in the sample by correlating the glucose detected.

8-11. (canceled)

12. The method of claim 1, wherein the PGM uses a test strip comprising glucose oxidase.

13. A sensor, comprising:
a solid support to which is attached:
one or more starting products, wherein at least one of the starting products can be converted to glucose-1-phosphate by a target enzyme, comprises an enzyme involved in the production of glucose-1-phosphate, or combinations thereof; and
alkaline phosphatase (ALP);
wherein the one or more starting products and ALP are attached to different areas of the solid support.

14. The sensor of claim 13, wherein:
the target enzyme comprises galactose-1-phosphate uridylyltransferase (GALT) and the one or more starting products comprise UDP-glucose and galactose-1-phosphate;
the target enzyme comprises glycogen phosphorylase (GP) and the one or more starting products comprise glycogen and 5′-adenosine monophosphate (AMP);
the target enzyme comprises galactokinase and the one or more starting products comprise α-D-galactose, UDP-glucose, and GALT; or
the target enzyme comprises UDP-glucose 4-epimerase and the one or more starting products comprise UDP-galactose, galactose-1-phosphate and GALT.

15. A sensor, comprising:
(a) a solid support to which is attached:
a first antibody specific for a target agent, wherein the first antibody comprises ALP;
an immobilized second antibody specific for the target agent, wherein the first antibody and the second antibody bind to different epitopes of the target agent; and
glucose-1-phosphate,
wherein the first antibody, second antibody, and glucose-1-phosphate are attached to different areas of the solid support; or
(b) a solid support, comprising:
a first area having attached thereto a first antibody conjugated to a functional molecule specific for a target agent, an agent that specifically binds to the functional molecule, and ALP conjugated to the functional molecule;
a second area having immobilized thereto a second antibody specific for the target agent, wherein the first antibody and second antibody bind to different epitopes of the target agent; and
a third area having attached thereto glucose-1-phosphate; or
(c) a first solid support, comprising:
a first area having immobilized thereto an alkaline-phosphate-target conjugate or an alkaline-phosphate-antibody conjugate;
a second area having attached thereto an antibody or aptamer attached to a second solid support, wherein the antibody or aptamer is specific for a target agent, or having attached thereto a target attached to the second solid support;
a third area having attached thereto glucose-1-phosphate; and
optionally a fourth area that blocks transport of the second solid support.

16. (canceled)

17. The sensor of claim 15, wherein the first antibody and the second antibody are polyclonal antibodies, monoclonal antibodies, antibody fragments, or combinations thereof.

18. (canceled)

19. The sensor of claim 13, wherein the solid support or first solid support comprises a membrane and the second solid support comprises a magnetic bead.

20. The sensor of claim 13, further comprising a sample pad and an absorption pad.

21. The sensor of claim 13, wherein the sensor further comprises:
glucose oxidase attached to the solid support;
one or more pH neutralizing agents; or
both, wherein the glucose oxidase and pH neutralizing agents are attached to different areas of the solid support.

22. A lateral flow device comprising:
the sensor of claim 13.

23. The lateral flow device of claim 23, wherein the lateral flow device comprises:
a wicking pad;
one or more reagent pads comprising one or more sensors or portions thereof attached thereto;
one or more reaction pads/membrane; and
an absorption pad.

24. The lateral flow device of claim 23, wherein the one or more reagent pads each comprise at least a portion of the solid support of the sensors.

25. A kit comprising:
one or more sensors of claim 13; and
one or more of a buffer, a chart for correlating detected glucose level and amount of target agent present.

26. A microfluidic device comprising:
the sensor of claim 13.

27. A method for detecting a target agent, comprising:
contacting one or more sensors of claim 13, with a test sample under conditions sufficient to allow a target in the test sample to interact with the one or more starting products, under conditions wherein glucose-1-phosphate will form;
allowing the glucose-1-phosphate to interact with the ALP under conditions wherein glucose will form;
detecting the glucose with a PGM; and
determining that the target agent is present in the sample when glucose is detected.

28. A method for detecting a target agent, comprising:
contacting one or more sensors of claim 15 with a test sample under conditions sufficient to allow a target agent in the test sample to interact with
(a) the ALP-first antibody specific for the target agent, under conditions wherein an ALP-first antibody-target immune complex will form;
contacting the ALP-first antibody-target immune complex with the second antibody under conditions sufficient to allow the second antibody to specifically bind to the target agent and under conditions wherein an immobilized ALP-first antibody-target-second antibody immune complex will form;
contacting agents in the mobile phase with the glucose-1-phosphate under conditions sufficient to allow the glucose-1-phosphate to interact with ALP under conditions wherein glucose will form;
detecting the glucose with a PGM; and
determining that the target agent is present in the sample when glucose is not detected; or
(b) the first antibody specific for the target agent, and the agent that specifically binds to the functional molecule to bind to the functional molecule conjugated to the first antibody and to the ALP under conditions wherein a first complex of functional molecule first antibody-target agent—agent that specifically binds to the functional molecule—functional molecule—ALP complex will form;
contacting the first complex with the second antibody under conditions sufficient to allow the second antibody to specifically bind to the target agent under conditions wherein a second complex of functional molecule first antibody-target agent—agent that specifically binds to the functional molecule—functional molecule—ALP-second antibody complex will form, wherein the second complex is immobilized;
allowing agents in the mobile phase to interact with glucose-1-phosphate under conditions sufficient to allow the glucose-1-phosphate to interact with ALP under conditions wherein glucose will form;
allowing ALP in the mobile phase to convert the glucose-1-phosphate to glucose;
detecting the glucose with a PGM; and
determining that the target agent is present in the sample when glucose is not detected; or
(c) the antibody or aptamer attached to the second solid support or to the alkaline-phosphate-antibody conjugate;
allowing agents in a mobile phase to interact with glucose-1-phosphate under conditions sufficient to allow the glucose-1-phosphate to interact with ALP under conditions wherein glucose will form;
allowing ALP in the mobile phase to convert the glucose-1-phosphate to glucose;
detecting the glucose with a PGM; and
determining whether the target agent is present in the sample depending on the glucose detected.

29-30. (canceled)

31. A method for detecting a target agent, comprising
contacting one or more lateral flow devices of claim 22 with a sample under conditions sufficient to allow the target agent in the sample to flow through the lateral flow device and interact with
(a) the one or more starting products present on the lateral flow device;
generating glucose-1-phosphate on a first reaction pad present on the lateral flow device;
allowing the glucose-1-phosphate to flow through the lateral flow device and interact with the ALP present on the lateral flow device;
generating glucose on a second reaction pad present on the lateral flow device;
optionally allowing the glucose to flow through the lateral flow device and interact with pH neutralizing agents present on the lateral flow device;
detecting the glucose; and
determining that the target agent is present in the sample when glucose is detected; or
(b) the ALP-labeled first antibody specific for the target agent on a first reagent pad of lateral flow device under conditions sufficient to allow a complex to form between the target agent and the ALP-first antibody;
allowing the target agent-first antibody-ALP complex to flow through the lateral flow device and interact with the second antibody immobilized on a second reagent pad of the lateral flow device under conditions sufficient to allow the second antibody to specifically bind to the target agent-first antibody-ALP complex, thereby generating an immobilized second antibody-target agent-first antibody-ALP complex;
allowing agents in the mobile phase to flow through the lateral flow device and interact with glucose-1-phosphate present on a third reagent pad of the lateral flow device under conditions sufficient to allow the glucose-1-phosphate to interact with ALP under conditions wherein glucose will form on a reaction pad present on the lateral flow device;
optionally allowing the glucose to flow through the lateral flow device and interact with pH neutralizing agents present on a fourth reagent pad of the lateral flow device;
detecting the glucose with a PGM; and
determining that the target agent is present in the sample when glucose is not detected; or
(c) the first antibody specific for the target agent present on a first reagent pad on the lateral flow device, and the agent that specifically binds to the functional molecule to bind to the functional molecule on the first antibody and conjugated to the ALP under conditions wherein a first complex of functional molecule first antibody-target agent—agent that specifically binds to the functional molecule—functional molecule—ALP complex will form, wherein the agent that specifically binds to the functional molecule and functional molecule conjugated ALP are present on the first reagent pad;
allowing the first complex to flow through the lateral flow device and interact with the second antibody immobilized on a second reagent pad under conditions sufficient to allow the second antibody to specifically bind to the target agent under conditions wherein an immobilized second complex of functional molecule first antibody-target agent—agent that specifically binds to the functional molecule—functional molecule—ALP-second antibody complex will form;
allowing non-immobilized ALP to flow through the lateral flow device and interact with the glucose-1-phosphate present on an third reagent pad;
allowing the non-immobilized ALP to convert the glucose-1-phosphate to glucose on a reaction pad present on the lateral flow device;
optionally allowing the glucose to flow through the lateral flow device and interact with pH neutralizing agents present on the lateral flow device;
detecting the glucose with a PGM; and
determining that the target agent is present in the sample when glucose is not detected.

32-33. (canceled)

34. A method for detecting a target agent, comprising:
contacting one or more lateral flow devices of claim 22 with a test sample under conditions sufficient to allow a target agent in the test sample to flow through the lateral flow device and contact the first area;
allowing the sample and the alkaline-phosphate-target conjugate or the alkaline-phosphate-antibody conjugate to flow to the second area;
allowing the sample to interact with the antibody or aptamer attached to a second solid support or to interact with the ALP-antibody conjugate on a reagent pad on the lateral flow device under conditions wherein a complex of antibody or aptamer attached to a second solid support-target or a complex of ALP-antibody conjugate-target will form in or on the reagent pad;
allowing the ALP-target conjugate or the complex of ALP-antibody conjugate-target to flow through the lateral flow device and interact with the glucose-1-phosphate in the third area;
allowing the non-immobilized ALP to convert the glucose-1-phosphate to glucose on a reaction pad present on the lateral flow device;
optionally allowing the glucose to flow through the lateral flow device and interact with pH neutralizing agents present on the lateral flow device;
detecting the glucose with a PGM; and
determining that the target agent is present in the sample when glucose is detected.

35. The microfluidic device of claim 26, wherein the lateral flow device comprises:
a sample entry port;
a region containing buffers;
a region containing the one or more starting products;
one or more mixing chambers;
a region containing ALP;
a region containing one or more pH neutralizing agents; and
an exit port.

36. A kit comprising:
one or more microfluidic devices of claim 26; and
one or more of a buffer, a chart for correlating detected glucose level and amount of target agent present.

37. A method for detecting a target agent, comprising:
introducing a test sample into the sample entry port of the microfluidic device of claim 26;
allowing a target in the test sample to interact with the region containing buffers; and the region containing the one or more starting products
allowing glucose-1-phosphate to form in one of the one or more mixing chambers;
allowing glucose to form in one of the one or more mixing chambers;
allowing the glucose to interact with the region containing one or more pH neutralizing agents;
detecting the glucose with a PGM; and
determining that the target agent is present in the sample when glucose is detected.
